(19) United States
(12) Patent Application Publication
(10) Pub. No.: US 2024/0123078 A1
(43) Pub. Date: Apr. 18, 2024

(54) COMPOUNDS AND METHODS FOR MODULATING IMMUNE-RELATED PROTEINS

(71) Applicant: Vividion Therapeutics, Inc., San Diego, CA (US)

(72) Inventors: Matthew PATRICELLI, San Diego, CA (US); Gabriel SIMON, San Diego, CA (US)

(21) Appl. No.: 17/768,641

(22) PCT Filed: Oct. 15, 2020
(86) PCT No.: PCT/US2020/055690

§ 371 Date: Apr. 13, 2022

Related U.S. Application Data
(60) Provisional application No. 62/916,172, filed on Oct. 16, 2019.

(51) Int. Cl. A61K 47/64; A61K 31/167; A61K 31/444; A61K 31/47; A61K 31/496; C07K 14/47

(52) U.S. Cl. CPC A61K 47/645; A61K 31/167; A61K 31/444; A61K 31/47; A61K 31/496; C07K 14/47

(57) ABSTRACT

Disclosed herein are methods, pharmaceutical compositions for modulating T cell proteins. Also disclosed herein are methods, pharmaceutical compositions, and vaccines for modulating an immune response.

### CROSS REFERENCE

This application claims the benefit of U.S. Application No. 62/916,172, filed Oct. 16, 2019, which is hereby incorporated by reference in its entirety.

### BACKGROUND OF THE DISCLOSURE

Protein biosynthesis and degradation is a dynamic process which sustains normal cell metabolism. In some instances, production of new proteins modulate proliferation and differentiation of cells and upon completion, these protein are degraded through one of two proteolytic mechanisms, the lysosome degradation system or the ubiquitin proteasome pathway. In some cases, a majority of cellular proteins are degraded by the proteasome pathway, and the process is initiated via tagging of a ubiquitin.

The immune system is a complex network of responses and processes that protects an organism and enables the organism to fight against a foreign agent. In some instances, there are two types of immune response when presented with a foreign agent. In one instance, the immune system responds with a B cell mediated response (e.g., humoral response or antibody-mediated response) when foreign agents (e.g., antigens and/or pathogens) are present in the lymph or blood. In another instance, the immune system responds with a T cell mediated response (e.g., a cell-mediated response) when cells that display aberrant MHC markers are present. In some instances, both humoral response and cell-mediated response are triggered by a foreign agent when, e.g., both antigens and cells containing aberrant MHC markers are present.

### SUMMARY OF THE DISCLOSURE

Some embodiments relate to a method of modulating an immune response in a subject in need thereof, comprising administering to the subject a therapeutically effective amount of a small molecule fragment of Formula (I):

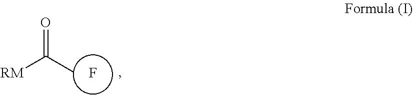

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct.

In some embodiments, the small molecule fragment is covalently bond to a cysteine residue of the cysteine-containing polypeptide.

In some embodiments, the cysteine-containing polypeptide-small molecule fragment adduct induces an immune response.

In some embodiments, the cysteine-containing polypeptide-small molecule fragment adduct induces a humoral immune response or a cell mediated immune response.

In some embodiments, the cysteine-containing polypeptide-small molecule fragment adduct increases an immune response relative to a control.

In some embodiments, the control is the level of an immune response in the subject prior to administration of the small molecule fragment or the level of an immune response in a subject who has not been exposed to the small molecule fragment.

In some embodiments, the cysteine-containing polypeptide is overexpressed in a disease or condition.

In some embodiments, the cysteine-containing polypeptide comprises one or more mutations, optionally overexpressed in a disease or condition.

In some embodiments, the disease or condition is cancer.

In some embodiments, the cysteine-containing polypeptide comprises a biologically active cysteine site, optionally located about 10 Å or less to an active-site ligand or residue.

In some embodiments, the cysteine-containing polypeptide is at most 50 amino acid residues in length.

In some embodiments, F is a small molecule fragment moiety illustrated in Table A.

In some embodiments, F is a fragment of the chloroacetamide compound in Table A after the chlorine (Cl) has been removed, or a fragment of the acrylamide compound after the C═C has been converted to an ethylene.

In some embodiments, the method modulates T cell activation.

In some embodiments, the method suppresses T cell activation.

In some embodiments, F optionally comprises a second reactive moiety.

In some embodiments, the method is an in vivo method.

In some embodiments, the endogenous cysteine-containing polypeptide is selected from proteins described in Tables 1 and 2.

In certain embodiments, described herein are compositions that comprise cysteine-containing proteins that are conjugated with a probe of Formula (II) or with a ligand disclosed herein. In some embodiments, disclosed herein is a protein-probe adduct wherein the probe binds to a cysteine residue illustrated in Tables 1 and 2; wherein the probe has a structure represented by Formula (II):

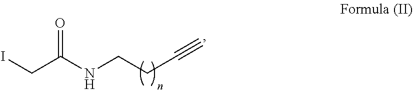

wherein n is 0-8.

In some embodiments, disclosed herein is a synthetic ligand that inhibits a covalent interaction between a protein and a probe, wherein in the absence of the synthetic ligand, the probe binds to a cysteine residue illustrated in Tables 1 and 2; and wherein the probe has a structure represented by Formula (I′):

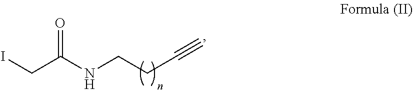

wherein n is 0-8.

In some embodiments, disclosed herein is a protein binding domain wherein said protein binding domain comprises a cysteine residue illustrated in Tables 1 and 2, wherein said cysteine forms an adduct with a compound of Formula (II):

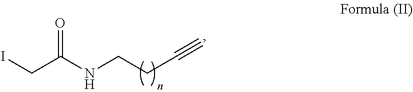

and wherein a compound of Formula (IIIA) or Formula (IIIB) interferes with the formation of the cysteine adduct by the compound of Formula (II), wherein Formula (IIIA) or Formula (IIIB) have the structure:

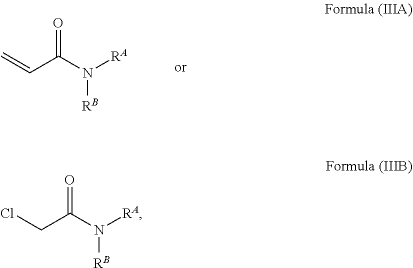

wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
or R^{A} and R^{B} together with the nitrogen to which they are attached form a 5, 6, 7 or 8-membered heterocyclic ring A, optionally having one additional heteroatom moiety independently selected from NW, O, or S; wherein A is optionally substituted; and
R¹ is independently H, D, substituted or unsubstituted C₁-C₆alkyl, substituted or unsubstituted

C₁-C₆fluoroalkyl, substituted or unsubstituted C₁-C₆heteroalkyl, substituted or unsubstituted aryl, or substituted or unsubstituted heteroaryl.

In certain embodiments, disclosed herein include methods, pharmaceutical compositions, and vaccines for modulating an immune response. In some embodiments, included herein are methods of administrating a small molecule fragment described herein for modulating an immune response. In additional embodiments, described herein are pharmaceutical compositions and vaccines which comprise a small molecule fragment described herein for modulating an immune response.

Disclosed herein, in certain embodiments, is a method of modulating an immune response in a subject in need thereof, comprising administering to the subject a therapeutically effective amount of a small molecule fragment of Formula (I):

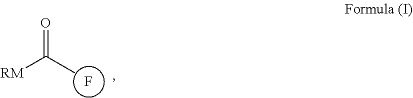

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some embodiments, the small molecule fragment is covalently bond to a cysteine residue of the cysteine-containing polypeptide. In some embodiments, the cysteine-containing polypeptide-small molecule fragment adduct induces an immune response. In some embodiments, the cysteine-containing polypeptide-small molecule fragment adduct induces a humoral immune response. In some embodiments, the cysteine-containing polypeptide-small molecule fragment adduct induces a cell mediated immune response. In some embodiments, the cysteine-containing polypeptide-small molecule fragment adduct increases an immune response relative to a control. In some embodiments, the cysteine-containing polypeptide-small molecule fragment adduct increases a humoral immune response relative to a control. In some embodiments, the cysteine-containing polypeptide-small molecule fragment adduct increases a cell mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell mediated immune response in the subject prior to administration of the small molecule fragment. In some embodiments, the control is the level of a humoral immune response or a cell mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease. In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the cysteine-containing polypeptide comprises a protein illustrated in Tables 1-2. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in Table A. In some embodiments, F further comprises a linker moiety that connects F to the carbonyl moiety. In some embodiments, the small molecule fragment is a small molecule fragment illustrated in Table A. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the method further comprises administering a cysteine-containing polypeptide-small molecule fragment adduct. In some embodiments, the cysteine-containing polypeptide is at most 50 amino acid residues in length. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence described in Example. In some embodiments, the method further comprises administration of an adjuvant. In some embodiments, the small molecule fragment is formulated for parenteral, oral, or intranasal administration. In some embodiments, the subject is a human.

Disclosed herein, in certain embodiments, is a vaccine comprising a small molecule fragment of Formula (I):

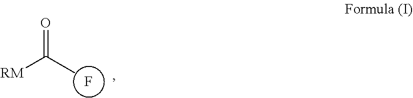

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with a cysteine-containing polypeptide to form a cysteine-containing polypeptide-small molecule fragment adduct. In some embodiments, the small molecule fragment is covalently bond to a cysteine residue of the cysteine-containing polypeptide. In some embodiments, the cysteine-containing polypeptide is an endogenous cysteine-containing polypeptide expressed in a subject. In some embodiments, administration of the small molecule fragment induces an immune response. In some embodiments, administration of the small molecule fragment induces a humoral immune response. In some embodiments, administration of the small molecule fragment induces a cell mediated immune response. In some embodiments, administration of the small molecule fragment increases an immune response relative to a control. In some embodiments, administration of the small molecule fragment increases a humoral immune response relative to a control. In some embodiments, administration of the small molecule fragment increases a cell mediated immune response relative to a control. In some embodiments, the control is the level of an immune response in the subject prior to administration of the small molecule fragment. In some embodiments, the control is the level of an immune response in a subject who has not been exposed to the small molecule fragment. In some embodiments, the control is the level of a humoral immune response or a cell mediated immune response in the subject prior to administration of the small molecule fragment. In some embodiments, the control is the level of a humoral immune response or a cell mediated immune response in a subject who has not been exposed to the small molecule fragment. In some embodiments, the cysteine-containing polypeptide is overexpressed in a disease or condition. In some embodiments, the cysteine-containing polypeptide comprises one or more mutations. In some embodiments, the cysteine-containing polypeptide comprising one or more mutations is overexpressed in a disease or condition. In some embodiments, the disease or condition is cancer. In some embodiments, the cysteine-containing polypeptide is a cancer-associated protein. In some embodiments, the cysteine-containing polypeptide is overexpressed in a cancer. In some embodiments, the cysteine-containing polypeptide comprising one or more mutations is overexpressed in a cancer. In some embodiments, the cysteine-containing polypeptide is a non-denatured form of the polypeptide. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide comprises a protein illustrated in Examples and Tables 1 and 2. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in Table A. In some embodiments, F further comprises a linker moiety that connects F to the carbonyl moiety. In some embodiments, the small molecule fragment is a small molecule fragment illustrated in Table A. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the vaccine further comprises a cysteine-containing polypeptide-small molecule fragment adduct. In some embodiments, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence described in Examples. In some embodiments, the vaccine further comprises an adjuvant. In some embodiments, the vaccine is formulated for parenteral, oral, or intranasal administration. Disclosed herein, in certain embodiments, is a pharmaceutical composition comprising:
a) a cysteine-containing polypeptide covalently bond to a small molecule fragment, wherein the small molecule fragment is a small molecule fragment of Formula (I):

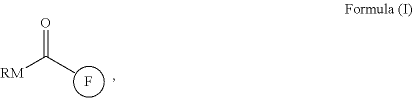

wherein:
RM is a reactive moiety selected from a Michael acceptor moiety, a leaving group moiety, or a moiety capable of forming a covalent bond with the thiol group of a cysteine residue; and
F is a small molecule fragment moiety; and
wherein the small molecule fragment is covalently bond to a cysteine residue of the cysteine-containing polypeptide; and
b) an excipient.

In some embodiments, the cysteine-containing polypeptide is a non-denatured form of the polypeptide. In some embodiments, the cysteine-containing polypeptide comprises a biologically active cysteine site. In some embodiments, the biologically active cysteine site is a cysteine residue that is located about 10 Å or less to an active-site ligand or residue. In some embodiments, the cysteine residue that is located about 10 Å or less to the active-site ligand or residue is an active site cysteine. In some embodiments, the biologically active cysteine site is an active site cysteine. In some embodiments, the biologically active cysteine site is a cysteine residue that is located greater than 10 Å from an active-site ligand or residue. In some embodiments, the cysteine residue that is located greater than 10 Å from the active-site ligand or residue is a non-active site cysteine. In some embodiments, the biologically active cysteine site is a non-active site cysteine. In some embodiments, the cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing polypeptide is an enzyme, a transporter, a receptor, a channel protein, an adaptor protein, a chaperone, a signaling protein, transcription related protein, or translation related protein. In some embodiments, the enzyme comprises kinases, proteases, or deubiquitinating enzymes. In some embodiments, the protease is a cysteine protease. In some embodiments, the cysteine protease comprises caspases. In some embodiments, the signaling protein comprises vascular endothelial growth factor. In some embodiments, the signaling protein comprises a redox signaling protein. In some embodiments, the cysteine-containing polypeptide is about 20, 30, 35, 40, 45, 50, 55, 60, 65, 70, 75, 80, 85, 90, 95, 100 amino acid residues in length or more. In some embodiments, the cysteine-containing polypeptide comprises a protein illustrated in Tables 1-2. In some embodiments, the cysteine-containing polypeptide comprises an isolated and purified protein. In some embodiments, the isolated and purified protein is a protein illustrated in Tables 1-2. In some embodiments, the cysteine-containing polypeptide is at most 50 amino acid residues in length. In some embodiments, the cysteine-containing polypeptide comprises an isolated and purified polypeptide comprising at least 80% sequence identity to at least seven contiguous amino acids of an amino acid sequence described in Example. In some embodiments, the cysteine-containing polypeptide comprises an isolated and purified polypeptide comprising at least 85% sequence identity to at least seven contiguous amino acids of an amino acid sequence described in Example. In some embodiments, the cysteine-containing polypeptide comprises an isolated and purified polypeptide comprising at least 90% sequence identity to at least seven contiguous amino acids of an amino acid sequence described in Example. In some embodiments, the cysteine-containing polypeptide comprises an isolated and purified polypeptide comprising at least 95% sequence identity to at least seven contiguous amino acids of an amino acid sequence described in Examples. In some embodiments, the cysteine-containing polypeptide comprises an isolated and purified polypeptide comprising at least 96% sequence identity to at least seven contiguous amino acids of an amino acid sequence described in Examples. In some embodiments, the cysteine-containing polypeptide comprises an isolated and purified polypeptide comprising at least 97% sequence identity to at least seven contiguous amino acids of an amino acid sequence described in Examples. In some embodiments, the cysteine-containing polypeptide comprises an isolated and purified polypeptide comprising at least 98% sequence identity to at least seven contiguous amino acids of an amino acid sequence described in Example. In some embodiments, the cysteine-containing polypeptide comprises an isolated and purified polypeptide comprising at least 99% sequence identity to at least seven contiguous amino acids of an amino acid sequence described in Example. In some embodiments, the cysteine-containing polypeptide comprises an isolated and purified polypeptide comprising 100% sequence identity to at least seven contiguous amino acids of an amino acid sequence described in Example. In some embodiments, the cysteine-containing polypeptide comprises an isolated and purified polypeptide consisting of 100% sequence identity to at least seven contiguous amino acids of an amino acid sequence described in Example. In some embodiments, the Michael acceptor moiety comprises an alkene or an alkyne moiety. In some embodiments, the covalent bond is formed between a portion of the Michael acceptor moiety on the small molecule fragment and a portion of a cysteine residue of the cysteine-containing polypeptide. In some embodiments, F is obtained from a compound library. In some embodiments, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library. In some embodiments, F is a small molecule fragment moiety illustrated in Table A. In some embodiments, F further comprises a linker moiety that connects F to the carbonyl moiety. In some embodiments, the small molecule fragment is a small molecule fragment illustrated in Table A. In some embodiments, the small molecule fragment has a molecular weight of about 150 Dalton or higher. In some embodiments, the small molecular fragment has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of the small molecular fragment is prior to enrichment with a halogen, a nonmetal, or a transition metal. In some embodiments, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher. In some embodiments, the pharmaceutical composition is formulated for parenteral, oral, or intranasal administration.

Disclosed herein, in certain embodiments, is a vaccine comprising a pharmaceutical composition disclosed above. In some embodiments, the vaccine further comprises an adjuvant. In some embodiments, the vaccine is formulated for parenteral, oral, or intranasal administration.

Disclosed herein, in certain embodiments, is an isolated and purified antibody or its binding fragment thereof comprising a heavy chain CDR1, CDR2 and CDR3 sequence and a light chain CDR1, CDR2 and CDR3 sequence, wherein the heavy chain and light chain CDRs interact with a cysteine-containing polypeptide that is covalently bond to a small molecule fragment, wherein the small molecule fragment is a small molecule fragment of Formula (I):

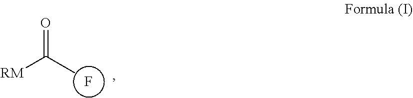

wherein:
RM is a reactive moiety selected from a Michael acceptor moiety, a leaving group moiety, or a moiety capable of forming a covalent bond with the thiol group of a cysteine residue; and
wherein the small molecule fragment is covalently bond to a cysteine residue of the cysteine-containing polypeptide.

In some embodiments, the antibody or its binding fragment thereof comprises a humanized antibody or binding fragment thereof, chimeric antibody or binding fragment thereof, monoclonal antibody or binding fragment thereof, monovalent Fab′, divalent Fab2, single-chain variable fragment (scFv), diabody, minibody, nanobody, single-domain antibody (sdAb), or camelid antibody or binding fragment thereof.

Disclosed herein, in certain embodiments, is a kit comprising a pharmaceutical composition described above.

Disclosed herein, in certain embodiments, is a kit comprising an isolated and purified antibody or its binding fragment thereof disclosed above.

### DETAILED DESCRIPTION OF THE DISCLOSURE

Cysteine containing proteins encompass a large repertoire of proteins that participate in numerous cellular functions such as mitogenesis, proliferation, apoptosis, gene regulation, and proteolysis. These proteins include enzymes, transporters, receptors, channel proteins, adaptor proteins, chaperones, signaling proteins, plasma proteins, transcription related proteins, translation related proteins, mitochondrial proteins, or cytoskeleton related proteins. Dysregulated expression of a cysteine containing protein, in many cases, is associated with or modulates a disease, for example, such as cancer.

In some instances, small molecule compounds are capable of eliciting an immune response. In some instances, these small molecule compounds are referred to as haptens. In some cases, a hapten is a non-immunogenic compound but becomes immunogenic when it interacts with a carrier molecule such as a protein. For example, upon administration of a small molecule hapten, the hapten forms an adduct with a protein of interest in a process refers to as haptenization. In some cases, the protein-hapten adduct becomes antigenically active and enables priming of T cells and B cells, thereby directing immune response to a cell that expresses the protein of interest.

In some embodiments, disclosed herein are small molecule fragments that elicit an immune response upon interaction with cysteine-containing proteins (or cysteine-containing polypeptides). In some instances, also disclosed herein includes use of a small molecule fragment described herein to elicit or modulate an immune response in a subject. In such instances, the small molecule fragment forms an adduct with an endogenous cysteine-containing protein, and subsequently directs immune response to the cell that expresses the endogenous cysteine-containing protein. In some instances, the cell that expresses the endogenous cysteine-containing protein is a disease cell (e.g., a cancerous cell). In some instances, the endogenous cysteine-containing protein is present only in a diseased cell (e.g., a cancerous cell). In other instances, the endogenous cysteine-containing protein is overexpressed in a diseased cell (e.g., a cancerous cell) and/or comprises one or more mutations in a diseased cell (e.g., a cancerous cell).

In some embodiments, also disclosed herein are vaccines and pharmaceutical compositions that comprise one or more small molecule fragments described herein. In some instances, additionally descried herein are vaccines and pharmaceutical compositions that comprise one or more cysteine-containing polypeptide-small molecule fragment adducts or antibodies that recognize a cysteine-containing polypeptide-small molecule fragment adduct described herein.

In additional embodiments, described herein include kits for use with any of the methods, vaccines, and pharmaceutical compositions disclosed herein.

### Small Molecule Fragments

In some embodiments, described herein include pharmaceutical compositions, vaccines, and methods of use of a small molecule fragment. In some embodiments, a small molecule fragment described herein comprises a non-naturally occurring molecule. In some instances, the non-naturally occurring molecule does not include a natural and/or non-natural peptide fragment, or a small molecule that is produced naturally within the body of a mammal.

In some embodiments, the molecule weight of a small molecule fragment described herein is the molecule weight prior to enrichment with one or more elements selected from a halogen, a nonmetal, a transition metal, or a combination thereof. In some embodiments, the molecule weight of a small molecule fragment described herein is the molecule weight prior to enrichment with a halogen. In some embodiments, the molecule weight of a small molecule fragment described herein is the molecule weight prior to enrichment with a nonmetal. In some embodiments, the molecule weight of a small molecule fragment described herein is the molecule weight prior to enrichment with a transition metal.

In some embodiments, a small molecule fragment described herein comprises micromolar or millimolar binding affinity. In some instances, a small molecule fragment comprises a binding affinity of about 1 μM, 10 μM, 100 μM, 500 μM, 1 mM, 10 mM, or higher.

In some embodiments, a small molecule fragment described herein has a high ligand efficiency (LE). Ligand efficiency is the measurement of the binding energy per atom of a ligand to its binding partner. In some instances, the ligand efficiency is defined as the ratio of the Gibbs free energy (ΔG) to the number of non-hydrogen atoms of the compound (N):

LE=(ΔG)/N.

In some cases, LE is also arranged as:

LE=1.4(−logIC₅₀)/N.

In some instances, the LE score is about 0.3 kcal mol⁻1HA⁻1, about 0.35 kcal mol⁻1HA⁻1, about 0.4 kcal mol⁻1HA⁻1, or higher.

In some embodiments, a small molecule fragment described herein is designed based on the Rule of 3. In some embodiments, the Rule of 3 comprises a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, a small molecule fragment described herein comprises three cyclic rings or less.

In some embodiments, a small molecule fragment described herein binds to a cysteine residue of a polypeptide that is about 20 amino acid residues in length or more. In some instances, a small molecule fragment described herein binds to a cysteine residue of a polypeptide that is about 30, 35, 40, 45, 50, 55, 60, 65, 70, 75, 80, 85, 90, 95, 100 amino acid residues in length or more.

In some embodiments, a small molecule fragment described herein further comprises pharmacokinetic parameters that are unsuitable as a therapeutic agent for administration without further optimization of the small molecule fragments. In some instances, the pharmacokinetic parameters that are suitable as a therapeutic agent comprise parameters in accordance with FDA guideline, or in accordance with a guideline from an equivalent Food and Drug Administration outside of the United States. In some instances, the pharmacokinetic parameters comprise the peak plasma concentration (Cmax), the lowest concentration of a therapeutic agent (Cmin), volume of distribution, time to reach Cmax, elimination half-life, clearance, and the life. In some embodiments, the pharmacokinetic parameters of the small molecule fragments are outside of the parameters set by the FDA guideline, or by an equivalent Food and Drug Administration outside of the United States. In some instances, a skilled artisan understands in view of the pharmacokinetic parameters of the small molecule fragments described herein that these small molecule fragments are unsuited as therapeutic agents without further optimization.

In some embodiments, a small molecule fragment described herein comprises a reactive moiety which forms a covalent interaction with the thiol group of a cysteine residue of a cysteine-containing protein, and an affinity handle moiety.

In some instances, a small molecule fragment described herein is a small molecule fragment of Formula (I):

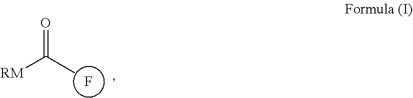

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some instances, the Michael acceptor moiety comprises an alkene or an alkyne moiety. In some cases, F is obtained from a compound library. In some cases, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some embodiments, a small molecule fragment of Formula (I) selectively interact with one or more protein variants. In some instances, for example, a small molecule fragment of Formula (I) interacts or binds to the wild-type protein but does not bind to a mutant form of the protein. Conversely, in some instances, a small molecule fragment of Formula (I) interacts or binds to one specific protein mutant but does not interact with either the wild-type or the same protein comprising a different mutation. As used herein, the term “variant” comprises mutations within the protein sequence, additions or deletions of the protein sequence, and/or termini truncations. As used herein, the term “variant” comprises a protein having different conformations, for example, an active conformation or an inactive conformation. In some instances, a small molecule fragment of Formula (I) interacts with about 1, 2, 3, 4, 5, or more different variants of a protein of interest. In additional instances, a small molecule fragment of Formula (I) interacts with about 1 variant of a protein of interest. In additional instances, a small molecule fragment of Formula (I) interacts with about 2 variants of a protein of interest. In additional instances, a small molecule fragment of Formula (I) interacts with about 3 variants of a protein of interest. In additional instances, a small molecule fragment of Formula (I) interacts with about 4 variants of a protein of interest. In additional instances, a small molecule fragment of Formula (I) interacts with about 5 variants of a protein of interest.

In some embodiments, a small molecule fragment of Formula (I) does not contain a second binding site. In some instances, a small molecule fragment moiety does not bind to the protein. In some cases, a small molecule fragment moiety does not covalently bind to the protein. In some instances, a small molecule fragment moiety does not interact with a secondary binding site on the protein. In some instances, the secondary binding site is an active site such as an ATP binding site. In some cases, the active site is at least about 10, 15, 20, 25, 35, 40 Å, or more away from the biologically active cysteine residue. In some instances, the small molecule fragment moiety does not interact with an active site such as an ATP binding site.

In some instances, F is a small molecule fragment moiety illustrated in Table A. In some cases, F further comprises a linker moiety that connects F to the carbonyl moiety. In some cases, the small molecule fragment is a small molecule fragment illustrated in Table A.

In some instances, F is a small molecule fragment moiety selected from: N-(4-bromophenyl)-N-phenylacrylamide, N-(1-benzoylpiperidin-4-yl)-2-chloro-N-phenylacetamide, 1-(4-benzylpiperidin-1-yl)-2-chloroethan-1-one, N-(2-(1H-indol-3-yl)ethyl)-2-chloroacetamide, N-(3,5-bis(trifluoromethyl)phenyl)acrylamide, N-(4-phenoxy-3-(trifluoromethyl)phenyl)-N-(pyridin-3-ylmethyl)acrylamide, N-(3,5-bis(trifluoromethyl)phenyl)acetamide, 2-chloro-1-(4-(hydroxydiphenylmethyl)piperidin-1-yl)ethan-1-one, (E)-3-(3,5-bis(trifluoromethyl)phenyl)-2-cyanoacrylamide, N-(3,5-bis(trifluoromethyl)phenyl)-2-bromopropanamide, N-(3,5-bis(trifluoromethyl)phenyl)-2-chloropropanamide, N-(3,5-bis(trifluoromethyl)phenyl)-N-(pyridin-3-ylmethyl)acrylamide, 3-(2-chloroacetamido)-5-(trifluoromethyl)benzoic acid, 1-(4-(5-fluorobenzisoxazol-3-yl)piperidin-1-yl)prop-2-en-1-one, tent-butyl 4-(4-acrylamido-2,6-difluorophenyl)piperazine-1-carboxylate, N-(4-bromo-2,5-dimethylphenyl)acrylamide, 2-Chloroacetamido-2-deoxy-α/β-D-glucopyranose, 2-chloro-1-(2-methyl-3,4-dihydroquinolin-1(2H)-yl)ethan-1-one, N-cyclohexyl-N-phenylacrylamide, 1-(5-bromoindolin-1-yl)prop-2-en-1-one, N-(1-benzylpiperidin-4-yl)-N-phenylacrylamide, 2-chloro-N-(2-methyl-5-(trifluoromethyl)phenyl)acetamide, 1-(5-bromoindolin-1-yl)-2-chloroethan-1-one, 2-chloro-N-(quinolin-5-yl)acetamide, 1-(4-benzylpiperidin-1-yl)prop-2-en-1-one, 2-chloro-N-((3-hydroxy-5-(hydroxymethyl)-2-methylpyridin-4-yl)methyl)acetamide, or 1-(6,7-dimethoxy-3,4-dihydroisoquinolin-2(1H)-yl)prop-2-en-1-one.

In some embodiments, the small molecule fragment of Formula (I) comprise a molecule weight of about 100, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 500, 550, 600, 650, 700, 750, 800, 850, 900, 950, 1000 Dalton, or higher. In some instances, the molecule weight of the small molecule fragment of Formula (I) is between about 150 and about 500, about 150 and about 450, abut 150 and about 440, about 150 and about 430, about 150 and about 400, about 150 and about 350, about 150 and about 300, about 150 and about 250, about 170 and about 500, about 180 and about 450, about 190 and about 400, about 200 and about 350, about 130 and about 300, or about 120 and about 250 Dalton.

In some embodiments, the molecule weight of the small molecule fragment of Formula (I) is the molecule weight prior to enrichment with one or more elements selected from a halogen, a nonmetal, a transition metal, or a combination thereof. In some embodiments, the molecule weight of the small molecule fragment of Formula (I) is the molecule weight prior to enrichment with a halogen. In some embodiments, the molecule weight of the small molecule fragment of Formula (I) is the molecule weight prior to enrichment with a nonmetal. In some embodiments, the molecule weight of the small molecule fragment of Formula (I) is the molecule weight prior to enrichment with a transition metal.

In some instances, the small molecule fragment of Formula (I) comprises micromolar or millimolar binding affinity. In some instances, the small molecule fragment of Formula (I) comprises a binding affinity of about 1 μM, 10 μM, 100 μM, 500 μM, 1 mM, 10 mM, or higher.

In some cases, the small molecule fragment of Formula (I) has a LE score about 0.3 kcal mol⁻1HA⁻1, about 0.35 kcal mol⁻1HA⁻1, about 0.4 kcal mol⁻1HA⁻1, or higher

In some embodiments, the small molecule fragment of Formula (I) follows the design parameters of Rule of 3. In some instances, the small molecule fragment of Formula (I) has a non-polar solvent-polar solvent (e.g. octanol-water) partition coefficient log P of about 3 or less, a molecular mass of about 300 Daltons or less, about 3 hydrogen bond donors or less, about 3 hydrogen bond acceptors or less, and about 3 rotatable bonds or less.

In some embodiments, the small molecule fragment of Formula (I) comprises three cyclic rings or less.

In some embodiments, the small molecule fragment of Formula (I) binds to a cysteine residue of a polypeptide (e.g., a cysteine-containing protein) that is about 20 amino acid residues in length or more. In some instances, the small molecule fragments described herein binds to a cysteine residue of a polypeptide (e.g., a cysteine-containing protein) that is about 30, 35, 40, 45, 50, 55, 60, 65, 70, 75, 80, 85, 90, 95, 100 amino acid residues in length or more.

In some instances, the small molecule fragment of Formula (I) has pharmacokinetic parameters outside of the parameters set by the FDA guideline, or by an equivalent Food and Drug Administration outside of the United States. In some instances, a skilled artisan understands in view of the pharmacokinetic parameters of the small molecule fragment of Formula (I) described herein that these small molecule fragment are unsuited as a therapeutic agent without further optimization.

TABLE A
Examples of electrophilic compounds

| Compound | Structure |
|---|---|
| KB-02 | 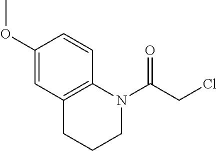 [SMILES: COc1ccc2c(c1)CCCN2C(=O)CCl] |
| KB-03 | 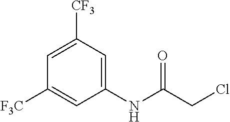 [SMILES: O=C(CCl)Nc1cc(C(F)(F)F)cc(C(F)(F)F)c1] |
| KB-05 | 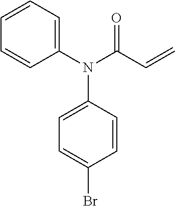 [SMILES: C=CC(=O)N(c1ccccc1)c1ccc(Br)cc1] |
| K4 | 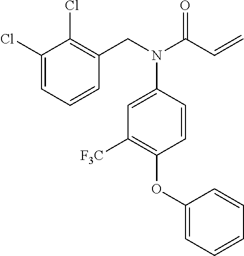 [SMILES: C=CC(=O)N(Cc1cccc(Cl)c1Cl)c1ccc(Oc2ccccc2)c(C(F)(F)F)c1] |
| L4 | 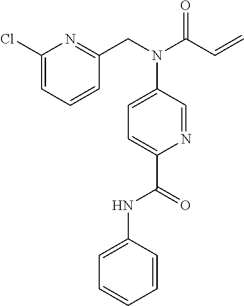 [SMILES: C=CC(=O)N(Cc1cccc(Cl)n1)c1ccc(C(=O)Nc2ccccc2)nc1] |
| B9 | 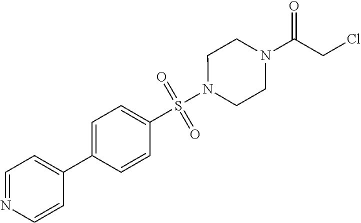 [SMILES: O=C(CCl)N1CCN(S(=O)(=O)c2ccc(-c3ccncc3)cc2)CC1] |

### Cysteine-Containing Proteins

In some embodiments, disclosed herein include a cysteine-containing polypeptide. In some instances, the cysteine-containing polypeptide is about 20, 30, 35, 40, 45, 50, 55, 60, 65, 70, 75, 80, 85, 90, 95, 100 amino acid residues in length or more. In some instances, the cysteine-containing polypeptide is a cysteine-containing protein or its fragment thereof. In some instances, the cysteine-containing protein is a soluble protein or its fragment thereof, or a membrane protein or its fragment thereof. In some instances, the cysteine-containing protein is involved in one or more of a biological process such as protein transport, lipid metabolism, apoptosis, transcription, electron transport, mRNA processing, or host-virus interaction. In some instances, the cysteine-containing protein is associated with one or more of diseases such as cancer or one or more disorders or conditions such as immune, metabolic, developmental, reproductive, neurological, psychiatric, renal, cardiovascular, or hematological disorders or conditions.

In some embodiments, the cysteine-containing protein comprises a biologically active cysteine residue. In some embodiments, the cysteine-containing protein comprises one or more cysteines in which at least one cysteine is a biologically active cysteine residue. In some cases, the biologically active cysteine site is a cysteine residue that is located about 10 Å or less to an active-site ligand or residue. In some cases, the cysteine residue that is located about 10 Å or less to the active-site ligand or residue is an active site cysteine. In other cases, the biologically active cysteine site is a cysteine residue that is located greater than 10 Å from an active-site ligand or residue. In some instances, the cysteine residue is located greater than 12 Å, 15 Å, 20 Å, 25 Å, 30 Å, 35 Å, 40 Å, 45 Å, or greater than 50 Å from an active-site ligand or residue. In some cases, the cysteine residue that is located greater than 10 Å from the active-site ligand or residue is a non-active site cysteine. In additional cases, the cysteine-containing protein exists in an active form, or in a pro-active form.

In some embodiments, the cysteine-containing protein comprises one or more functions of an enzyme, a transporter, a receptor, a channel protein, an adaptor protein, a chaperone, a signaling protein, a plasma protein, transcription related protein, translation related protein, mitochondrial protein, or cytoskeleton related protein. In some embodiments, the cysteine-containing protein is an enzyme, a transporter, a receptor, a channel protein, an adaptor protein, a chaperone, a signaling protein, a plasma protein, transcription related protein, translation related protein, mitochondrial protein, or cytoskeleton related protein. In some instances, the cysteine-containing protein has an uncategorized function.

In some embodiments, the cysteine-containing protein is an enzyme. An enzyme is a protein molecule that accelerates or catalyzes chemical reaction. In some embodiments, non-limiting examples of enzymes include kinases, proteases, or deubiquitinating enzymes.

In some instances, exemplary kinases include tyrosine kinases such as the TEC family of kinases such as Tec, Bruton's tyrosine kinase (Btk), interleukin-2-indicible T-cell kinase (Itk) (or Emt/Tsk), Bmx, and Txk/Rlk; spleen tyrosine kinase (Syk) family such as SYK and Zeta-chain-associated protein kinase 70 (ZAP-70); Src kinases such as Src, Yes, Fyn, Fgr, Lck, Hck, Blk, Lyn, and Frk; JAK kinases such as Janus kinase 1 (JAK1), Janus kinase 2 (JAK2), Janus kinase 3 (JAK3), and Tyrosine kinase 2 (TYK2); or ErbB family of kinases such as Her1 (EGFR, ErbB1), Her2 (Neu, ErbB2), Her3 (ErbB3), and Her4 (ErbB4).

In some embodiments, the cysteine-containing protein is a protease. In some embodiments, the protease is a cysteine protease. In some cases, the cysteine protease is a caspase. In some instances, the caspase is an initiator (apical) caspase. In some instances, the caspase is an effector (executioner) caspase. Exemplary caspase includes CASP2, CASP8, CASP9, CASP10, CASP3, CASP6, CASP7, CASP4, and CASP5. In some instances, the cysteine protease is a cathepsin. Exemplary cathepsin includes Cathepsin B, Cathepsin C, Cathepsin F, Cathepsin H, Cathepsin K, Cathepsin L1, Cathepsin L2, Cathepsin O, Cathepsin S, Cathepsin W, or Cathepsin Z.

In some embodiments, the cysteine-containing protein is a deubiquitinating enzyme (DUB). In some embodiments, exemplary deubiquitinating enzymes include cysteine proteases DUBs or metalloproteases. Exemplary cysteine protease DUBs include ubiquitin-specific protease (USP/UBP) such as USP1, USP2, USP3, USP4, USP5, USP6, USP7, USP8, USP9X, USP9Y, USP10, USP11, USP12, USP13, USP14, USP15, USP16, USP17, USP17L2, USP17L3, USP17L4, USP17L5, USP17L7, USP17L8, USP18, USP19, USP20, USP21, USP22, USP23, USP24, USP25, USP26, USP27X, USP28, USP29, USP30, USP31, USP32, USP33, USP34, USP35, USP36, USP37, USP38, USP39, USP40, USP41, USP42, USP43, USP44, USP45, or USP46; ovarian tumor (OTU) proteases such as OTUB1 and OTUB2; Machado-Josephin domain (MJD) proteases such as ATXN3 and ATXN3L; and ubiquitin C-terminal hydrolase (UCH) proteases such as BAP1, UCHL1, UCHL3, and UCHL5. Exemplary metalloproteases include the Jab1/Mov34/Mpr1 Pad1 N-terminal+ (MPN+) (JAMM) domain proteases.

In some embodiments, exemplary cysteine-containing proteins as enzymes include, but are not limited to, Glyceraldehyde-3-phosphate dehydrogenase (GAPDH), Protein arginine N-methyltransferase 1 (PRMT1), Peptidyl-prolyl cis-trans isomerase NIMA-interaction (PIN1), Acetyl-CoA acetyltransferase (mitochondrial) (ACAT1), Glutathione S-transferase P (GSTP1), Elongation factor 2 (EEF2), Glutathione S-transferase omega-1 (GSTO1), Acetyl-CoA acetyltransferase (mitochondrial) (ACAT1), Protein disulfide-isomerase A4 (PDIA4), Prostaglandin E synthase 3 (PTGES3), Adenosine kinase (ADK), Elongation factor 2 (EEF2), Isoamyl acetate-hydrolyzing esterase 1 homolog (IAH1), Peroxiredoxin-5 (mitochondrial) (PRDX5), Inosine-5-monophosphate dehydrogenase 2 (IMPDH2), 3-hydroxyacyl-CoA dehydrogenase type-2 (HSD17B10), Omega-amidase NIT2 (NIT2), Aldose reductase (AKR1B1), Monofunctional C1-tetrahydrofolate synthase (mitochondrial) (MTHFD1L), Protein disulfide-isomerase A6 (PDIA6), Pyruvate kinase isozymes M1/M2 (PKM), 6-phosphogluconolactonase (PGLS), Acetyl-CoA acetyltransferase (mitochondrial) (ACAT1), ERO1-like protein alpha (ERO1L), Thioredoxin domain-containing protein 17 (TXNDC17), Protein disulfide-isomerase A4 (PDIA4), Protein disulfide-isomerase A3 (PDIA3), 3-ketoacyl-CoA thiolase (mitochondrial) (ACAA2), Dynamin-2 (DNM2), DNA replication licensing factor MCM3 (MCM3), Serine-tRNA ligase (cytoplasmic) (SARS), Fatty acid synthase (FASN), Acetyl-CoA acetyltransferase (mitochondrial) (ACAT1), Protein disulfide-isomerase (P4HB), Deoxycytidine kinase (DCK), Eukaryotic translation initiation factor 3 subunit (EIF3F), Protein disulfide-isomerase A6 (PDIA6), UDP-N-acetylglucosamine-peptide N-acetylglucosamine (OGT), Ketosamine-3-kinase (FN3KRP), Protein DJ-1 (PARK7), Phosphoglycolate phosphatase (PGP), DNA replication licensing factor MCM6 (MCM6), Fructose-2,6-bisphosphatase TIGAR (TIGAR), Cleavage and polyadenylation specificity factor subunit (CPSF3), Ubiquitin-conjugating enzyme E2 L3 (UBE2L3), Alanine-tRNA ligase, cytoplasmic (AARS), Mannose-1-phosphate guanyltransferase alpha (GMPPA), C-1-tetrahydrofolate synthase (cytoplasmic) (MTHFD1), Dynamin-1-like protein (DNM1L), Protein disulfide-isomerase A3 (PDIA3), Aspartyl aminopeptidase (DNPEP), Acetyl-CoA acetyltransferase (cytosolic) (ACAT2), Thioredoxin domain-containing protein 5 (TXNDC5), Thymidine kinase (cytosolic) (TK1), Inosine-5-monophosphate dehydrogenase 2 (IMPDH2), Ubiquitin carboxyl-terminal hydrolase isozyme L3 (UCHL3), Integrin-linked protein kinase (ILK), Cyclin-dependent kinase 2 (CDK2), Histone acetyltransferase type B catalytic subunit (HAT1), Enoyl-CoA delta isomerase 2 (mitochondrial) (ECI2), C-1-tetrahydrofolate synthase (cytoplasmic) (MTHFD1), Deoxycytidine kinase (DCK), Ubiquitin-like modifier-activating enzyme 6 (UBA6), Protein-L-isoaspartate(D-aspartate) O-methyltransferase (PCMT1), Monofunctional C1-tetrahydrofolate synthase (mitochondrial) (MTHFD1L), Thymidylate kinase (DTYMK), Protein ETHE1 (mitochondrial) (ETHE1), Arginine-tRNA ligase (cytoplasmic) (RARS), NEDD8-activating enzyme E1 catalytic subunit (UBA3), Dual specificity mitogen-activated protein kinase (MAP2K3), Ubiquitin-conjugating enzyme E2S (UBE2S), Amidophosphoribosyltransferase (PPAT), Succinate-semialdehyde dehydrogenase (mitochondrial) (ALDH5A1), CAD, Phosphoenolpyruvate carboxykinase (PCK2), 6-phosphofructokinase type C (PFKP), Acyl-CoA synthetase family member 2 (mitochondrial) (ACSF2), Multifunctional protein ADE2 (PAICS), Desumoylating isopeptidase 1 (DESI1), 6-phosphofructokinase type C (PFKP), V-type proton ATPase catalytic subunit A (ATP6V1A), 3-ketoacyl-CoA thiolase (peroxisomal) (ACAA1), Galactokinase (GALK1), Thymidine kinase (cytosolic) (TK1), ATPase WRNIP1 (WRNIP1), Phosphoribosylformylglycinamidine synthase (PFAS), V-type proton ATPase catalytic subunit A (ATP6V1A), Thioredoxin domain-containing protein 5 (TXNDC5), 4-trimethylaminobutyraldehyde dehydrogenase (ALDH9A1), Dual specificity mitogen-activated protein kinase (MAP2K4), Calcineurin-like phosphoesterase domain-containing (CPPED1), Dual specificity protein phosphatase 12 (DUSP12), Phosphoribosylformylglycinamidine synthase (PFAS), Diphosphomevalonate decarboxylase (MVD), D-3-phosphoglycerate dehydrogenase (PHGDH), Cell cycle checkpoint control protein RAD9A (RAD9A), Peroxiredoxin-1 (PRDX1), Sorbitol dehydrogenase (SORD), Peroxiredoxin-4 (PRDX4), AMP deaminase 2 (AMPD2), Isocitrate dehydrogenase (IDH1), Pyruvate carboxylase (mitochondrial) (PC), Integrin-linked kinase-associated serine/threonine (ILKAP), Methylmalonate-semialdehyde dehydrogenase (ALDH6A1), 26S proteasome non-ATPase regulatory subunit 14 (PSMD14), Thymidylate kinase (DTYMK), 6-phosphofructo-2-kinase/fructose-2,6-bisphosphata (PFKFB2), Peroxiredoxin-5 (mitochondrial) (PRDXS), PDP1, Cathepsin B (CTSB), Transmembrane protease serine 12 (TMPRSS12), UDP-glucose 6-dehydrogenase (UGDH), Histidine triad nucleotide-binding protein 1 (HINT1), E3 ubiquitin-protein ligase UBRS (UBRS), SAM domain and HD domain-containing protein 1 (SAMHD1), Probable tRNA threonylcarbamoyladeno sine biosynthesis (OSGEP), Methylated-DNA-protein-cysteine methyltransferase (MGMT), Fatty acid synthase (FASN), Adenosine deaminase (ADA), Cyclin-dependent kinase 19 (CDK19), Serine/threonine-protein kinase 38 (STK38), Mitogen-activated protein kinase 9 (MAPK9), tRNA (adenine(58)-N(1))-methyltransferase catalytic (TRMT61A), Glyoxylate reductase/hydroxypyruvate reductase (GRHPR), Aldehyde dehydrogenase (mitochondrial) (ALDH2), Mitochondrial-processing peptidase subunit beta (PMPCB), 3-ketoacyl-CoA thiolase, peroxisomal (ACAA1), Lysophosphatidic acid phosphatase type 6 (ACP6), Ubiquitin/ISG15-conjugating enzyme E2 L6 (UBE2L6), Caspase-8 (CASP8), 2,5-phosphodiesterase 12 (PDE12), Thioredoxin domain-containing protein 12 (TXNDC12), Nitrilase homolog 1 (NIT1), ERO1-like protein alpha (ERO1L), SUMO-activating enzyme subunit 1 (SAE1), Leucine-tRNA ligase (cytoplasmic) (LARS), Protein-glutamine gamma-glutamyltransferase 2 (TGM2), Probable DNA dC-dU-editing enzyme APOBEC-3C (APOBEC3C), Double-stranded RNA-specific adenosine deaminase (ADAR), Isocitrate dehydrogenase (IDH2), Methylcrotonoyl-CoA carboxylase beta chain (mitochondrial) (MCCC2), Uridine phosphorylase 1 (UPP1), Glycogen phosphorylase (brain form) (PYGB), E3 ubiquitin-protein ligase UBR5 (UBR5), Procollagen-lysine,2-oxoglutarate 5-dioxygenase 1 (PLOD1), Ubiquitin carboxyl-terminal hydrolase 48 (USP48), Aconitate hydratase (mitochondrial) (ACO2), GMP reductase 2 (GMPR2), Pyrroline-5-carboxylate reductase 1 (mitochondrial) (PYCR1), Cathepsin Z (CTSZ), E3 ubiquitin-protein ligase UBR2 (UBR2),Cysteine protease ATG4B (ATG4B), Serine/threonine-protein kinase Nek9 (NEK9), Lysine-specific demethylase 4B (KDM4B), Insulin-degrading enzyme (IDE), Dipeptidyl peptidase 9 (DPP9), Decaprenyl-diphosphate synthase subunit 2 (PDSS2), TFIIH basal transcription factor complex helicase (ERCC3), Methionine-R-sulfoxide reductase B2 (mitochondrial) (MSRB2), E3 ubiquitin-protein ligase BRE1B (RNF40), Thymidylate synthase (TYMS), Cyclin-dependent kinase 5 (CDKS), Bifunctional 3-phosphoadenosine 5-phosphosulfate (PAPS S2), Short/branched chain specific acyl-CoA dehydrogenase (ACADSB), Cathepsin D (CTSD), E3 ubiquitin-protein ligase HUWE1 (HUWE1), Calpain-2 catalytic subunit (CAPN2), Dual specificity mitogen-activated protein kinase (MAP2K7), Mitogen-activated protein kinase kinase kinase MLT (MLTK), Bleomycin hydrolase (BLMH), Probable ATP-dependent RNA helicase DDX59 (DDX59), Cystathionine gamma-lyase (CTH), S-adenosylmethionine synthase isoform type-2 (MAT2A), 6-phosphofructokinase type C (PFKP), Cytidine deaminase (CDA), DNA-directed RNA polymerase II subunit RPB2 (POLR2B), Protein disulfide-isomerase (P4HB), Procollagen-lysine,2-oxoglutarate 5-dioxygenase 3 (PLOD3), Nucleoside diphosphate-linked moiety X motif 8 (mitochondrial) (NUDT8), E3 ubiquitin-protein ligase HUWE1 (HUWE1), Methylated-DNA-protein-cysteine methyltransferase (MGMT), Nitrilase homolog 1 (NIT1), Interferon regulatory factor 2-binding protein 1 (IRF2BP1), Ubiquitin carboxyl-terminal hydrolase 16 (USP16), Glycylpeptide N-tetradecanoyltransferase 2 (NMT2), Cyclin-dependent kinase inhibitor 3 (CDKN3), Hydroxysteroid dehydrogenase-like protein 2 (HSDL2), Serine/threonine-protein kinase VRK1 (VRK1), Serine/threonine-protein kinase A-Raf (ARAF), ATP-citrate synthase (ACLY), Probable ribonuclease ZC3H12D (ZC3H12D), Peripheral plasma membrane protein CASK (CASK), DNA polymerase epsilon subunit 3 (POLE3), Aldehyde dehydrogenase X (mitochondrial) (ALDH1B1), UDP-N-acetylglucosamine transferase subunit ALG13 (ALG13), Protein disulfide-isomerase A4 (PDIA4), DNA polymerase alpha catalytic subunit (POLA1), Ethylmalonyl-CoA decarboxylase (ECHDC1), Protein-tyrosine kinase 2-beta (PTK2B), E3 SUMO-protein ligase RanBP2 (RANBP2), Legumain (LGMN), Non-specific lipid-transfer protein (SCP2), Long-chain-fatty-acid-CoA ligase 4 (ACSL4), Dual specificity protein phosphatase 12 (DUSP12), Oxidoreductase HTATIP2 (HTATIP2), Serine/threonine-protein kinase MRCK beta (CDC42BPB), Histone-lysine N-methyltransferase EZH2 (EZH2), Non-specific lipid-transfer protein (SCP2), Dual specificity mitogen-activated protein kinase (MAP2K7), Ubiquitin carboxyl-terminal hydrolase 28 (USP28), 6-phosphofructokinase (liver type) (PFKL), SWI/SNF-related matrix-associated actin-dependent (SMARCAD1), Protein phosphatase methylesterase 1 (PPME1), DNA replication licensing factor MCM5 (MCM5), 6-phosphofructo-2-kinase/fructose-2,6-bisphosphata (PFKFB4), Dehydrogenase/reductase SDR family member 11 (DHRS11), Pyroglutamyl-peptidase 1 (PGPEP1), Probable E3 ubiquitin-protein ligase (MYCBP2), DNA fragmentation factor subunit beta (DFFB), Deubiquitinating protein VCIP135 (VCPIP1), Putative transferase CAF17 (mitochondrial) (IBA57), Calpain-7 (CAPN7), GDP-L-fucose synthase (TSTA3), Protein disulfide-isomerase A4 (PDIA4, Probable ATP-dependent RNA helicase (DDX59), RNA exonuclease 4 (REXO4), PDK1, E3 SUMO-protein ligase (PIAS4), DNA (cytosine-5)-methyltransferase 1 (DNMT1), Alpha-aminoadipic semialdehyde dehydrogenase (ALDH7A1), Hydroxymethylglutaryl-CoA synthase (cytoplasmic) (HMGCS1), E3 ubiquitin-protein ligase (SMURF2), Aldehyde dehydrogenase X (mitochondrial) (ALDH1B1), Tyrosine-protein kinase (BTK), DNA repair protein RAD50 (RAD50), ATP-binding domain-containing protein 4 (ATPBD4), Nucleoside diphosphate kinase 3 (NME3), Interleukin-1 receptor-associated kinase 1 (IRAK1), Ribonuclease P/MRP protein subunit POP5 (POP5), Peptide-N(4)-(N-acetyl-beta-glucosaminyl)asparagine (NGLY1), Caspase-2 (CASP2), Ribosomal protein S6 kinase alpha-3 (RPS6KA3), E3 ubiquitin-protein ligase UBR1 (UBR1), Serine/threonine-protein kinase Chk2 (CHEK2), Phosphatidylinositol 3,4,5-trisphosphate 5-phospha (INPPL1), Histone acetyltransferase p300 (EP300), Creatine kinase U-type (mitochondrial) (CKMT1B), E3 ubiquitin-protein ligase TRIM33 (TRIM33), Cancer-related nucleoside-triphosphatase (NTPCR), Aconitate hydratase (mitochondrial) (ACO2), Ubiquitin carboxyl-terminal hydrolase 34 (USP34), Probable E3 ubiquitin-protein ligase HERC4 (HERC4), E3 ubiquitin-protein ligase HECTD1 (HECTD1), Peroxisomal 2,4-dienoyl-CoA reductase (DECR2), Helicase ARIP4 (RAD54L2), Ubiquitin-like modifier-activating enzyme 7 (UBA7), ER degradation-enhancing alpha-mannosidase-like 3 (EDEM3), Ubiquitin-conjugating enzyme E2O (UBE2O), Dual specificity mitogen-activated protein kinase (MAP2K7), Myotubularin-related protein 1 (MTMR1), Calcium-dependent phospholipase A2 (PLA2G5), Mitotic checkpoint serine/threonine-protein kinase (BUB1B), Putative transferase CAF17 (mitochondrial) (IBA57), Tyrosine-protein kinase ZAP-70 (ZAP70), E3 ubiquitin-protein ligase pellino homolog 1 (PELI1), Neuropathy target esterase (PNPLA6), Ribosomal protein S6 kinase alpha-3 (RPS6KA3), N6-adenosine-methyltransferase 70 kDa subunit (METTL3), Fructosamine-3-kinase (FN3K), Ubiquitin carboxyl-terminal hydrolase 22 (USP22), Rab3 GTPase-activating protein catalytic subunit (RAB3GAP1), Caspase-5 (CASP5), L-2-hydroxyglutarate dehydrogenase (mitochondrial) (L2HGDH), Saccharopine dehydrogenase-like oxidoreductase (SCCPDH), FLAD1 FAD synthase, Lysine-specific demethylase 3A (KDM3A), or Ubiquitin carboxyl-terminal hydrolase 34 (USP34).

In some embodiments, the cysteine-containing protein is a signaling protein. In some instances, exemplary signaling protein includes vascular endothelial growth factor (VEGF) proteins or proteins involved in redox signaling. Exemplary VEGF proteins include VEGF-A, VEGF-B, VEGF-C, VEGF-D, and PGF. Exemplary proteins involved in redox signaling include redox-regulatory protein FAM213A.

In some embodiments, the cysteine-containing protein is a transcription factor or regulator. Exemplary cysteine-containing proteins as transcription factors and regulators include, but are not limited to, 40S ribosomal protein S3 (RPS3), Basic leucine zipper and W2 domain-containing protein (BZW1), Poly(rC)-binding protein 1 (PCBP1), 40S ribosomal protein S11 (RPS11), 40S ribosomal protein S4, X isoform (RPS4X), Signal recognition particle 9 kDa protein (SRP9), Non-POU domain-containing octamer-binding protein (NONO), N-alpha-acetyltransferase 15, NatA auxiliary subunit (NAA15), Cleavage stimulation factor subunit 2 (CSTF2), Lamina-associated polypeptide 2, isoform alpha (TMPO), Heterogeneous nuclear ribonucleoprotein R (FINRNPR), MMS19 nucleotide excision repair protein homolog (MMS19), SWI/SNF complex subunit SMARCC2 (SMARCC2), Enhancer of mRNA-decapping protein 3 (EDC3), H/ACA ribonucleoprotein complex subunit 2 (NHP2), WW domain-containing adapter protein with coiled-c (WAC), N-alpha-acetyltransferase 15 NatA auxiliary subunit (NAA15), 40S ribosomal protein S11 (RPS11), Signal transducer and activator of transcription 1 (STAT1), Mediator of RNA polymerase II transcription subunit (MED15), Lamina-associated polypeptide 2 (isoform alpha) (TMPO), MMS19 nucleotide excision repair protein homolog (MMS19), DNA mismatch repair protein Msh2 (MSH2), Recombining binding protein suppressor of hairless (RBPJ), Mediator of RNA polymerase II transcription subunit (MED17), Heterogeneous nuclear ribonucleoprotein U (HNRNPU), Transcription initiation factor IIA subunit 2 (GTF2A2), Chromatin accessibility complex protein 1 (CHRAC1), CDKN2A-interacting protein (CDKN2AIP), Zinc finger protein 217 (ZNF217), Signal transducer and activator of transcription 3 (STAT3), WD repeat and HMG-box DNA-binding protein 1 (WDHD1), Lamina-associated polypeptide 2 (isoform alpha) (TMPO), Lamina-associated polypeptide 2 (isoforms beta/gam) (TMPO), Interferon regulatory factor 4 (IRF4), Protein flightless-1 homolog (FLII), Heterogeneous nuclear ribonucleoprotein F (HNRNPF), Nucleus accumbens-associated protein 1 (NACC1), Transcription elongation regulator 1 (TCERG1), Protein HEXIM1 (HEXIM1), Enhancer of mRNA-decapping protein (EDC3), Zinc finger protein Aiolos (IKZF3), Transcription elongation factor SPT5 (SUPT5H), Forkhead box protein K1 (FOXK1), LIM domain-containing protein 1 (LIMD1), MMS19 nucleotide excision repair protein homolog (MMS19), Elongator complex protein 4 (ELP4), Ankyrin repeat and KH domain-containing protein 1 (ANKHD1), PML, Nuclear factor NF-kappa-B p100 subunit (NFKB2), Heterogeneous nuclear ribonucleoprotein L-like (HNRPLL), CCR4-NOT transcription complex subunit 3 (CNOT3), Constitutive coactivator of PPAR-gamma-like protein (FAM120A), Mediator of RNA polymerase II transcription subunit (MED15), 60S ribosomal protein L7 (RPL7), Interferon regulatory factor 8 (IRF8), COUP transcription factor 2 (NR2F2), Mediator of RNA polymerase II transcription subunit (MEDI), tRNA (uracil-5-)-methyltransferase homolog A (TRMT2A), Transcription factor p65 (RELA), Exosome complex component RRP42 (EXOSC7), General transcription factor 3C polypeptide 1 (GTF3C1), Mothers against decapentaplegic homolog 2 (SMAD2), Ankyrin repeat domain-containing protein 17 (ANKRD17), MMS19 nucleotide excision repair protein homolog (MMS19), Death domain-associated protein 6 (DAXX), Zinc finger protein 318 (ZNF318), Thioredoxin-interacting protein (TXNIP), Glucocorticoid receptor (NR3C1), Iron-responsive element-binding protein 2 (IREB2), Zinc finger protein 295 (ZNF295), Polycomb protein SUZ12 (SUZ12), Cleavage stimulation factor subunit 2 tau variant (CSTF2T), C-myc promoter-binding protein (DENND4A), Pinin (PNN), Mediator of RNA polymerase II transcription subunit (MED9), POU domain, class 2, transcription factor 2 (POU2F2), Enhancer of mRNA-decapping protein 3 (EDC3), A-kinase anchor protein 1 (mitochondrial) (AKAP1), Transcription factor RelB (RELB), RNA polymerase II-associated protein 1 (RPAP1), Zinc finger protein 346 (ZNF346), Chromosome-associated kinesin KIF4A (KIF4A), Mediator of RNA polymerase II transcription subunit (MED12), Protein NPAT (NPAT), Leucine-rich PPR motif-containing protein (mitochondrial) (LRPPRC), AT-hook DNA-binding motif-containing protein 1 (AHDC1), Mediator of RNA polymerase II transcription subunit (MED12), Bromodomain-containing protein 8 (BRD8), Trinucleotide repeat-containing gene 6B protein (TNRC6B), Aryl hydrocarbon receptor nuclear translocator (ARNT), Activating transcription factor 7-interacting protein (ATF7IP), Glucocorticoid receptor (NR3C1), Chromosome transmission fidelity protein 18 homolog (CHTF18), or C-myc promoter-binding protein (DENND4A).

In some embodiments, the cysteine-containing protein is a channel, transporter or receptor. Exemplary cysteine-containing proteins as channels, transporters, or receptors include, but are not limited to, Chloride intracellular channel protein 4 (CLIC4), Exportin-1 (XPO1), Thioredoxin (TXN), Protein SEC13 homolog (SEC13), Chloride intracellular channel protein 1 (CLIC1), Guanine nucleotide-binding protein subunit beta-2 (GNB2L1), Sorting nexin-6 (SNX6), Conserved oligomeric Golgi complex subunit 3 (COG3), Nuclear cap-binding protein subunit 1 (NCBP1), Cytoplasmic dynein 1 light intermediate chain 1 (DYNC1LI1), MOB-like protein phocein (MOB4), Programmed cell death 6-interacting protein (PDCD6IP), Glutaredoxin-1 (GLRX), ATP synthase subunit alpha (mitochondrial) (ATP5A1), Treacle protein (TCOF1), Dynactin subunit 1 (DCTN1), Importin-7 (IPO7), Exportin-2 (CSE1L), ATP synthase subunit gamma (mitochondrial) (ATP5C1), Trafficking protein particle complex subunit 5 (TRAPPCS), Thioredoxin mitochondrial (TXN2), THO complex subunit 6 homolog (THOC6), Exportin-1 (XPO1), Nuclear pore complex protein Nup50 (NUP50), Treacle protein (TCOF1), Nuclear pore complex protein Nup93 (NUP93), Nuclear pore glycoprotein p62 (NUP62), Cytoplasmic dynein 1 heavy chain 1 (DYNC1H1), Thioredoxin-like protein 1 (TXNL1), Nuclear pore complex protein Nup214 (NUP214), Protein lin-7 homolog C (LIN7C), ADP-ribosylation factor-binding protein GGA2 (GGA2), Trafficking protein particle complex subunit 4 (TRAPPC4), Protein quaking (QKI), Perilipin-3 (PLIN3), Copper transport protein ATOX1 (ATOX1), Unconventional myosin-Ic (MYO1C), Nucleoporin NUP53 (NUP35), Vacuolar protein sorting-associated protein 18 homolog (VPS18), Dedicator of cytokinesis protein 7 (DOCK7), Nucleoporin p54 (NUP54), Ras-related GTP-binding protein C (RRAGC), Arf-GAP with Rho-GAP domain (ANK repeat and PH domain) (ARAP1), Exportin-5 (XPO5), Kinectin (KTN1), Chloride intracellular channel protein 6 (CLIC6), Voltage-gated potassium channel subunit beta-2 (KCNAB2), Exportin-5 (XPO5), Ras-related GTP-binding protein C (RRAGC), Ribosome-binding protein 1 (RRBP1), Acyl-CoA-binding domain-containing protein 6 (ACBD6), Chloride intracellular channel protein 5 (CLIC5), Pleckstrin homology domain-containing family A member (PLEKHA2), ADP-ribosylation factor-like protein 3 (ARL3), Protein transport protein Sec24C (SEC24C), Voltage-dependent anion-selective channel protein (VDAC3), Programmed cell death 6-interacting protein (PDCD6IP), Chloride intracellular channel protein 3 (CLIC3), Multivesicular body subunit 12A (FAM125A), Eukaryotic translation initiation factor 4E transporter (EIF4ENIF1), NmrA-like family domain-containing protein 1 (NMRAL1), Nuclear pore complex protein Nup98-Nup96 (NUP98), Conserved oligomeric Golgi complex subunit 1 (COG1), Importin-4 (IPO4), Pleckstrin homology domain-containing family A member (PLEKHA2), Cytoplasmic dynein 1 heavy chain 1 (DYNC1H1), DENN domain-containing protein 1C (DENND1C), Cytoplasmic dynein 1 heavy chain 1 (DYNC1H1), Protein ELYS (AHCTF1), Trafficking protein particle complex subunit 1 (TRAPPC1), Guanine nucleotide-binding protein-like 3 (GNL3), or Importin-13 (IPO13).

In some embodiments, the cysteine-containing protein is a chaperone. Exemplary cysteine-containing proteins as chaperones include, but are not limited to, 60 kDa heat shock protein (mitochondrial) (HSPD1), T-complex protein 1 subunit eta (CCT7), T-complex protein 1 subunit epsilon (CCT5), Heat shock 70 kDa protein 4 (HSPA4), GrpE protein homolog 1 (mitochondrial) (GRPEL1), Tubulin-specific chaperone E (TBCE), Protein unc-45 homolog A (UNC45A), Serpin H1 (SERPINH1), Tubulin-specific chaperone D (TBCD), Peroxisomal biogenesis factor 19 (PEX19), BAG family molecular chaperone regulator 5 (BAG5), T-complex protein 1 subunit theta (CCT8), Protein canopy homolog 3 (CNPY3), DnaJ homolog subfamily C member 10 (DNAJC10), ATP-dependent Clp protease ATP-binding subunit clp (CLPX), or Midasin (MDN1).

In some embodiments, the cysteine-containing protein is an adapter, scaffolding or modulator protein. Exemplary cysteine-containing proteins as adapter, scaffolding, or modulator proteins include, but are not limited to, Proteasome activator complex subunit 1 (PSME1), TIP41-like protein (TIPRL), Crk-like protein (CRKL), Cofilin-1 (CFL1), Condensin complex subunit 1 (NCAPD2), Translational activator GCN1 (GCN1L1), Serine/threonine-protein phosphatase 2A 56 kDa regulatory (PPP2R5D), UPF0539 protein C7orf59 (C7orf59), Protein diaphanous homolog 1 (DIAPH1), Protein asunder homolog (Asun), Ras GTPase-activating-like protein IQGAP1 (IQGAP1), Sister chromatid cohesion protein PDS5 homolog A (PDS5A), Reticulon-4 (RTN4), Proteasome activator complex subunit 4 (PSME4), Condensin complex subunit 2 (NCAPH), Sister chromatid cohesion protein PDS5 homolog A (PDSPDS5A), cAMP-dependent protein kinase type I-alpha regulatory (PRKAR1A), Host cell factor 1 (HCFC1), Serine/threonine-protein phosphatase 4 regulatory (PPP4R2), Apoptotic chromatin condensation inducer in the nucleus (ACIN1), BRISC and BRCA1-A complex member 1 (BABAM1), Interferon-induced protein with tetratricopeptide (IFIT3), Ras association domain-containing protein 2 (RASSF2), Hsp70-binding protein 1 (HSPBP1), TBC1 domain family member 15 (TBC1D15), Dynamin-binding protein (DNMBP), Condensin complex subunit 1 (NCAPD2), Beta-2-syntrophin (SNTB2), Disks large homolog 1 (DLG1), TBC1 domain family member 13 (TBC1D13), Formin-binding protein 1-like (FNBP1L), Translational activator GCN1 (GCN1L1), GRB2-related adapter protein (GRAP), G2/mitotic-specific cyclin-B1 (CCNB1), Myotubularin-related protein 12 (MTMR12), Protein FADD (FADD), Translational activator GCN1 (GCN1L1), Wings apart-like protein homolog (WAPAL), cAMP-dependent protein kinase type II-beta regulatory (PRKAR2B), Malcavernin (CCM2), MPP1 55 kDa erythrocyte membrane protein, Actin filament-associated protein 1 (AFAP1), Tensin-3 (TNS3), tRNA methyltransferase 112 homolog (TRMT112), Symplekin (SYMPK), TBC1 domain family member 2A (TBC1D2), ATR-interacting protein (ATRIP), Ataxin-10 (ATXN10), Succinate dehydrogenase assembly factor 2 (mitochondrial) (SDHAF2), Formin-binding protein 1 (FNBP1), Myotubularin-related protein 12 (MTMR12), Interferon-induced protein with tetratricopeptide (IFIT3), Protein CBFA2T2 (CBFA2T2), Neutrophil cytosol factor 1 (NCF1), or Protein syndesmos (NUDT16L1).

In some embodiments, exemplary cysteine-containing immune-related proteins include, but are not limited to, MYD88, TRAF2, RIPK1, TAB3, TAK1, MYSM1, USP4, OTUD78, USP4, CYLD, NFKB1, ZAP70, PTPN11, PD-1, PLCG1, PRKC1, BIRC3, MAP4K1, BCL10, MALT1, IRBKB, CHUK, UBASH31, TNFAIP3, DGKZ, DGKA, CBLB, PDPK1, MAP3K8, PTPN22, PCBP2, or RHOH. In some embodiments, the cysteine-containing protein is involved in redox regulation. In some instances, the cysteine-containing protein is TXN, GSR, P4HB, PDIA6, GSTO1, or ERO1L. In some instances, the cysteine is TXN_C32, TXN_C35, GSR_C102, P4HB_C53, P4HB_C56, P4HB_C397, P4HB_C400, PDIA3_C57, PDIA3_C60, PDIA3_C406, PDIA3_C409, PDIA6_C55, PDIA6_C58, PDIA6_C190, PDIA6_C193, GSTO1_C32, ERO1L_C35, or ERO1L_C37. In some embodiments, the cysteine-containing protein is involved in metal binding. In some instances, the cysteine-containing protein is ADH5, LCP1, LMO7, TRIM65, or NDUFS1. In some instances, the cysteine is ADH5_C45, ADH5_C60, LCP1_C42, LCP1_C101, LMO7_C1646, TRIM65_C06, or NDUFS1_C78. In some embodiments, the cysteine-containing protein is involved in DNA/RNA-binding. In some instances, the cysteine-containing protein is UPF0361, RPP30, or MBD4. In some instances, the cysteine is UPF0361_07, RPP30_C225, MBD4_C61, MBD4_C82, or MBD4_C88. In some embodiments, the cysteine-containing protein is involved in cofactor binding. In some embodiments, the cysteine-containing protein is involved in metabolite binding. In some instances, the cysteine-containing protein is GIMAP2, G6PD, MMUT, ORC1, PAPSS1, or IDH1. In some instances, the cysteine is GIMAP2_C175, G6PD_C385, MMUT_C742, ORC1_C506, PAPSS1_C156, or IDHl1_C269. In some instances, liganding a protein with methods of the present invention leads to degradation. In one instance, the NuRD complex is liganded and degraded by the proteasome. In some instances, the cysteine-containing protein is a transcription factor. In some instances, the cysteine-containing protein is an adaptor. In some instances, the cysteine-containing protein is MALT1, IRF9, TAPBP, TAP1, TAP2, NLRP1, TMEM173, FADD, PRKDC, IRS1, RAD50, IKBKB, PSTPIP1, TRAF3IP2, BCL10, AP3D1, MYD88, IRF4, IRF8, RELB, STAT1, STAT2, STAT3, STAT4, NFKBIL1, NFKB1, NFKB2, IKZF1, BCL11B, FOXP3, RBPJ, RNF113A, GFI1, RFX5, or ERCC3. In some instances, the immune-related cysteine is DOCK2_C1083, MALT1_C71, ADA_C75, PSMB8_C120, PSMB8_C124, STAT1_C492, TRNT1_C373, IRF9_C319, CECR1_C408, TYK2_C838, TAPBP_C440, ALG3_C21, TMEM173_C91, ATM_C532, ATM_C1396, CASP8_C360, CTSC_C255, CTSC_C258, IRAK4_C13, MYD88_C203, PSTPIP1_C305, STT3A_C193, TAP2_C641, IKBKB_C464, NHEJ1_C74, PRKDC_C4045, OR ERCC3_C342. In some instances, the probe reacts with an immune-enriched protein. In some instances, the cysteine is ADPGK_C40, APOBEC3C_C130, BIRC3_C28, BIRC6_C213, CD38_C287, CD38_C296, CD7_C219, CMTM7_C12, DCTN4_C258, DDX60_C1051, DDX60_C517, DESI1_C108, GNLY_C43, GNLY_C45, IL16_C1004, IL16_C1011, INPP4A_C275, INPP4A_C286, INPP5D_C1088, JAK1_C810, JAK1_C817, JAK2_C243, KEAP1_C288, KIAA0317_C382, MCM3_C119, MCM3_C123, MCM3_C126, MRPL39_C133, MTM41_C117, NARF_C99, NCAPD2_C767, NPC1_C816, PARP10_C981, PIK3R6_C709, PIK3R6_C713, PPP1R12A_C553, PREX1_C1266, PSMA1_C148, PSMA1_C156, RABGAP1L_C248, RHOH_C108, RNF213_C4407, RRM1_C492, SLC12A9_C911, SON_C92, SYNE2_C1032, SYNE2_C568, TXNIP_C170, UBASH3A_C435, UBE2L6_C86, UBE2L6_C98, USP7_C315, UVRAG_C239, or ZDHHC18_C156. In some instances, the cysteine-containing protein is MBD2, MBD3, GATAD2B, MTA2, FAM21B, GATAD2A, HDAC2, HDAC1, MTA1, RBBP4, CHD4, RBBP7, CHD3. In some instances, the cysteine is CHD3_C1997, CHD4_C1594, GATAD2A_C417, GATAD2A_C420, GATAD2A_426, GATAD21_C417 and C420, GATAD2B_C308, GATAD2B_C423, HDAC1_100, HDAC1_110, HDAC1_100 and HDAC1_110, HDAC1_C408, HDAC1_C261, MBD2_C10, MBD2_C11, MBD2_C10 AND MBD2_C11, MBD2_C359, MBD3_C266, MBD3_C215, or MTA2_C209.

### Polypeptides Comprising a Cysteine Interacting Site

In some embodiments, a cysteine-containing polypeptide comprises a polypeptide that is at most 50 amino acid residues in length. In some embodiments, a cysteine-containing polypeptide comprises an isolated and purified polypeptide comprising at least 70% sequence identity to at least seven contiguous amino acids of an amino acid sequence selected from SEQs disclosed in the Examples. In some embodiments, a cysteine-containing polypeptide comprises an isolated and purified polypeptide comprising at least 75% sequence identity to at least seven contiguous amino acids of an amino acid sequence selected from SEQs disclosed in the Examples. In some embodiments, a cysteine-containing polypeptide comprises an isolated and purified polypeptide comprising at least 80% sequence identity to at least seven contiguous amino acids of an amino acid sequence selected from SEQs disclosed in the Examples. In some embodiments, a cysteine-containing polypeptide comprises an isolated and purified polypeptide comprising at least 85% sequence identity to at least seven contiguous amino acids of an amino acid sequence selected from SEQs disclosed in the Examples. In some embodiments, a cysteine-containing polypeptide comprises an isolated and purified polypeptide comprising at least 90% sequence identity to at least seven contiguous amino acids of an amino acid sequence selected from SEQs disclosed in the Examples. In some embodiments, a cysteine-containing polypeptide comprises an isolated and purified polypeptide comprising at least 91% sequence identity to at least seven contiguous amino acids of an amino acid sequence selected from SEQs disclosed in the Examples. In some embodiments, a cysteine-containing polypeptide comprises an isolated and purified polypeptide comprising at least 92% sequence identity to at least seven contiguous amino acids of an amino acid sequence selected from SEQs disclosed in the Examples. In some embodiments, a cysteine-containing polypeptide comprises an isolated and purified polypeptide comprising at least 93% sequence identity to at least seven contiguous amino acids of an amino acid sequence selected from SEQs disclosed in the Examples. In some embodiments, a cysteine-containing polypeptide comprises an isolated and purified polypeptide comprising at least 94% sequence identity to at least seven contiguous amino acids of an amino acid sequence selected from SEQs disclosed in the Examples. In some embodiments, a cysteine-containing polypeptide comprises an isolated and purified polypeptide comprising at least 95% sequence identity to at least seven contiguous amino acids of an amino acid sequence selected from SEQs disclosed in the Examples. In some embodiments, a cysteine-containing polypeptide comprises an isolated and purified polypeptide comprising at least 96% sequence identity to at least seven contiguous amino acids of an amino acid sequence selected from SEQs disclosed in the Examples. In some embodiments, a cysteine-containing polypeptide comprises an isolated and purified polypeptide comprising at least 97% sequence identity to at least seven contiguous amino acids of an amino acid sequence selected from SEQs disclosed in the Examples. In some embodiments, a cysteine-containing polypeptide comprises an isolated and purified polypeptide comprising at least 98% sequence identity to at least seven contiguous amino acids of an amino acid sequence selected from SEQs disclosed in the Examples. In some embodiments, a cysteine-containing polypeptide comprises an isolated and purified polypeptide comprising at least 99% sequence identity to at least seven contiguous amino acids of an amino acid sequence selected from SEQs disclosed in the Examples. In some embodiments, a cysteine-containing polypeptide comprises an isolated and purified polypeptide comprising 100% sequence identity to at least seven contiguous amino acids of an amino acid sequence selected from SEQs disclosed in the Examples. In some embodiments, a cysteine-containing polypeptide comprises an isolated and purified polypeptide consisting of 100% sequence identity to at least seven contiguous amino acids of an amino acid sequence selected from SEQs disclosed in the Examples.

As used herein, a polypeptide includes natural amino acids, unnatural amino acids, or a combination thereof. In some instances, an amino acid residue refers to a molecule containing both an amino group and a carboxyl group. Suitable amino acids include, without limitation, both the D- and L-isomers of the naturally-occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

The term “α-amino acid” refers to a molecule containing both an amino group and a carboxyl group bound to a carbon which is designated the α-carbon.

The term “β-amino acid” refers to a molecule containing both an amino group and a carboxyl group in β configuration.

“Naturally occurring amino acid” refers to any one of the twenty amino acids commonly found in peptides synthesized in nature, and known by the one letter abbreviations A, R, N, C, D, Q, E, G, H, I, L, K, M, F, P, S, T, W, Y and V.

“Hydrophobic amino acids” include small hydrophobic amino acids and large hydrophobic amino acids. “Small hydrophobic amino acid” are glycine, alanine, proline, and analogs thereof. “Large hydrophobic amino acids” are valine, leucine, isoleucine, phenylalanine, methionine, tryptophan, and analogs thereof. “Polar amino acids” are serine, threonine, asparagine, glutamine, cysteine, tyrosine, and analogs thereof “Charged amino acids” are lysine, arginine, histidine, aspartate, glutamate, and analogs thereof.

The term “amino acid analog” refers to a molecule which is structurally similar to an amino acid and which is substituted for an amino acid in the formation of a peptidomimetic macrocycle Amino acid analogs include, without limitation, β-amino acids and amino acids where the amino or carboxy group is substituted by a similarly reactive group (e.g., substitution of the primary amine with a secondary or tertiary amine, or substitution of the carboxy group with an ester).

The term “non-natural amino acid” refers to an amino acid which is not one of the twenty amino acids commonly found in peptides synthesized in nature, and known by the one letter abbreviations A, R, N, C, D, Q, E, G, H, I, L, K, M, F, P, S, T, W, Y and V.

In some instances, amino acid analogs include β-amino acid analogs. Examples of β-amino acid analogs include, but are not limited to, the following: cyclic β-amino acid analogs; β-alanine; (R)-β-phenylalanine; (R)-1,2,3,4-tetrahydro-isoquinoline-3-acetic acid; (R)-3-amino-4-(1-naphthyl)-butyric acid; (R)-3-amino-4-(2,4-dichlorophenyl)butyric acid; (R)-3-amino-4-(2-chlorophenyl)-butyric acid; (R)-3-amino-4-(2-cyanophenyl)-butyric acid; (R)-3-amino-4-(2-fluorophenyl)-butyric acid; (R)-3-amino-4-(2-furyl)-butyric acid; (R)-3-amino-4-(2-methylphenyl)-butyric acid; (R)-3-amino-4-(2-naphthyl)-butyric acid; (R)-3-amino-4-(2-thienyl)-butyric acid; (R)-3-amino-4-(2-trifluoromethylphenyl)-butyric acid; (R)-3-amino-4-(3,4-dichlorophenyl)butyric acid; (R)-3-amino-4-(3,4-difluorophenyl)butyric acid; (R)-3-amino-4-(3-benzothienyl)-butyric acid; (R)-3-amino-4-(3-chlorophenyl)-butyric acid; (R)-3-amino-4-(3-cyanophenyl)-butyric acid; (R)-3-amino-4-(3-fluorophenyl)-butyric acid; (R)-3-amino-4-(3-methylphenyl)-butyric acid; (R)-3-amino-4-(3-pyridyl)-butyric acid; (R)-3-amino-4-(3-thienyl)-butyric acid; (R)-3-amino-4-(3-trifluoromethylphenyl)-butyric acid; (R)-3-amino-4-(4-bromophenyl)-butyric acid; (R)-3-amino-4-(4-chlorophenyl)-butyric acid; (R)-3-amino-4-(4-cyanophenyl)-butyric acid; (R)-3-amino-4-(4-fluorophenyl)-butyric acid; (R)-3-amino-4-(4-iodophenyl)-butyric acid; (R)-3-amino-4-(4-methylphenyl)-butyric acid; (R)-3-amino-4-(4-nitrophenyl)-butyric acid; (R)-3-amino-4-(4-pyridyl)-butyric acid; (R)-3-amino-4-(4-trifluoromethylphenyl)-butyric acid; (R)-3-amino-4-pentafluoro-phenylbutyric acid; (R)-3-amino-5-hexenoic acid; (R)-3-amino-5-hexynoic acid; (R)-3-amino-5-phenylpentanoic acid; (R)-3-amino-6-phenyl-5-hexenoic acid; (S)-1,2,3,4-tetrahydro-isoquinoline-3-acetic acid; (S)-3-amino-4-(1-naphthyl)-butyric acid; (S)-3-amino-4-(2,4-dichlorophenyl)butyric acid; (S)-3-amino-4-(2-chlorophenyl)-butyric acid; (S)-3-amino-4-(2-cyanophenyl)-butyric acid; (S)-3-amino-4-(2-fluorophenyl)-butyric acid; (S)-3-amino-4-(2-furyl)-butyric acid; (S)-3-amino-4-(2-methylphenyl)-butyric acid; (S)-3-amino-4-(2-naphthyl)-butyric acid; (S)-3-amino-4-(2-thienyl)-butyric acid; (S)-3-amino-4-(2-trifluoromethylphenyl)-butyric acid; (S)-3-amino-4-(3,4-dichlorophenyl)butyric acid; (S)-3-amino-4-(3,4-difluorophenyl)butyric acid; (S)-3-amino-4-(3-benzothienyl)-butyric acid; (S)-3-amino-4-(3-chlorophenyl)-butyric acid; (S)-3-amino-4-(4-cyanophenyl)-butyric acid; (S)-3-amino-4-(3-fluorophenyl)-butyric acid; (S)-3-amino-4-(3-methylphenyl)-butyric acid; (S)-3-amino-4-(3-pyridyl)-butyric acid; (S)-3-amino -4-(3-thienyl)-butyric acid; (S)-3-amino-4-(3-trifluoromethylphenyl)-butyric acid; (S)-3-amino-4-(4-bromophenyl)-butyric acid; (S)-3-amino-4-(4-chlorophenyl) butyric acid; (S)-3-amino-4-(4-cyanophenyl)-butyric acid; (S)-3-amino-4-(4-fluorophenyl) butyric acid; (S)-3-amino-4-(4-iodophenyl)-butyric acid; (S)-3-amino-4-(4-methylphenyl)-butyric acid; (S)-3-amino-4-(4-nitrophenyl)-butyric acid; (S)-3-amino-4-(4-pyridyl)-butyric acid; (S)-3-amino-4-(4-trifluoromethylphenyl)-butyric acid; (S)-3-amino-4-pentafluoro-phenylbutyric acid; (S)-3-amino-5-hexenoic acid; (S)-3-amino-5-hexynoic acid; (S)-3-amino-5-phenylpentanoic acid; (S)-3-amino-6-phenyl-5-hexenoic acid; 1,2,5,6-tetrahydropyridine-3-carboxylic acid; 1,2,5,6-tetrahydropyridine-4-carboxylic acid; 3-amino-3-(2-chlorophenyl)-propionic acid; 3-amino-3-(2-thienyl)-propionic acid; 3-amino-3-(3-bromophenyl)-propionic acid; 3-amino-3-(4-chlorophenyl)-propionic acid; 3-amino-3-(4-methoxyphenyl)-propionic acid; 3-amino-4,4,4-trifluoro -butyric acid; 3-aminoadipic acid; D-β-phenylalanine; β-leucine; L-β-homoalanine; L-β-homoaspartic acid γ-benzyl ester; L-β-homoglutamic acid δ-benzyl ester; L-β-homoisoleucine; L-β-homoleucine; L-β-homomethionine; L-β-homophenylalanine; L-β-homoproline; L-β-homotryptophan; L-β-homovaline; L-Nω-benzyloxycarbonyl-β-homolysine; Nω-L-β-homoarginine; O-benzyl-L-βL-β-homohydroxyproline; O-benzyl-L-β-homoserine; O-benzyl-L-β-homothreonine; O-benzyl -L-β-homotyrosine; γ-trityl-L-β-homoasparagine; (R)-β-phenylalanine; L-β-homoaspartic acid γ-t-butyl ester; L-β-homoglutamic acid γ-t-butyl ester; L-Nω-β-homolysine; Nδ-trityl-L-β-homoglutamine; Nω-2,2,4,6,7-pentamethyl-dihydrobenzofuran-5-sulfonyl-L-β-homoarginine; O-t-butyl-L-β-homohydroxy-proline; O-t-butyl-L -β-homoserine; O-t-butyl-L-β-homothreonine; O-t-butyl-L-β-homotyrosine; 2-aminocyclopentane carboxylic acid; and 2-aminocyclohexane carboxylic acid.

In some instances, amino acid analogs include analogs of alanine, valine, glycine or leucine. Examples of amino acid analogs of alanine, valine, glycine, and leucine include, but are not limited to, the following: α-methoxyglycine; α-allyl-L-alanine; α-aminoisobutyric acid; α-methyl-leucine; α-(1-naphthyl)-D-alanine; β-(1-naphthyl)-L-alanine; β-(2-naphthyl)-D-alanine; β-(2-naphthyl)-L-alanine; β-(2-pyridyl)-D-alanine; β-(2-pyridyl)-L-alanine; β-(2-thienyl)-D-alanine; β-(2-thienyl)-L-alanine; β-(3-benzothienyl)-D-alanine; β-(3-benzothienyl)-L-alanine; β-(3-pyridyl)-D-alanine; β-(3-pyridyl)-L-alanine; β-(4-pyridyl)-D-alanine; β-(4-pyridyl)-L-alanine; β-chloro-L-alanine; β-cyano-L-alanine; β-cyclohexyl-D-alanine; β-cyclohexyl-L-alanine; β-cyclopenten-1-yl-alanine; β-cyclopentyl-alanine; β-cyclopropyl-L -Ala-OH·dicyclohexylammonium salt; β-t-butyl-D-alanine; β-t-butyl-L-alanine; γ-aminobutyric acid; L-α,β-diaminopropionic acid; 2,4-dinitro-phenylglycine; 2,5-dihydro-D-phenylglycine; 2-amino-4,4,4-trifluorobutyric acid; 2-fluoro-phenylglycine; 3-amino-4,4,4-trifluoro-butyric acid; 3-fluoro-valine; 4,4,4-trifluoro-valine; 4,5-dehydro-L-leu-OH·dicyclohexylammonium salt; 4-fluoro-D-phenylglycine; 4-fluoro -L-phenylglycine; 4-hydroxy-D-phenylglycine; 5,5,5-trifluoro-leucine; 6-aminohexanoic acid; cyclopentyl-D-Gly-OH·dicyclohexylammonium salt; cyclopentyl-Gly-OH·dicyclohexylammonium salt; D-α,β-diaminopropionic acid; D-α-aminobutyric acid; D-α-t-butylglycine; D-(2-thienyl)glycine; D-(3-thienyl)glycine; D-2-aminocaproic acid; D-2-indanylglycine; D-allylglycine-dicyclohexylammonium salt; D-cyclohexylglycine; D-norvaline; D-phenylglycine; β-aminobutyric acid; β-aminoisobutyric acid; β-bromophenyl)glycine; (2-methoxyphenyl)glycine; (2-methylphenyl)glycine; (2-thiazoyl)glycine; (2-thienyl)glycine; 2-amino-3-(dimethylamino)-propionic acid; L-α,β-diaminopropionic acid; L-α-aminobutyric acid; L-α-t-butylglycine; L-(3-thienyl)glycine; L-2-amino-3-(dimethylamino)-propionic acid; L-2-aminocaproic acid dicyclohexyl-ammonium salt; L-2-indanylglycine; L-allylglycine·dicyclohexyl ammonium salt; L-cyclohexylglycine; L-phenylglycine; L-propargylglycine; L-norvaline; N-α-aminomethyl-L-alanine; D-α,γ-diaminobutyric acid; L-α,γ-diaminobutyric acid; β-cyclopropyl-L-alanine; (N-β-(2,4-dinitrophenyl))-L-α,β-diaminopropionic acid; (N-β-1-(4,4-dimethyl-2,6-dioxocyclohex-1-ylidene)ethyl)-D-α,β-diaminopropionic acid; (N-β-1-(4,4-dimethyl -2,6-dioxocyclohex-1-ylidene)ethyl)-L-α,β-diaminopropionic acid; (N-β-4-methyltrityl)-L-α,β-diaminopropionic acid; (N-β-allyloxycarbonyl)-L-α,β-diaminopropionic acid; (N-γ-1-(4,4-dimethyl-2,6-dioxocyclohex- 1-ylidene)ethyl)-D-α,γ-diaminobutyric acid; (N-γ-1-(4,4-dimethyl-2,6-dioxocyclohex-1-ylidene)ethyl)-L-α,γ-diaminobutyric acid; (N-γ-4-methyltrityl)-D-α,γ-diaminobutyric acid; (N-γ-4-methyltrityl)-L-α,γ-diaminobutyric acid; (N-γ-allyloxycarbonyl)-L-α,γ-diaminobutyric acid; D-α,γ-diaminobutyric acid; 4,5-dehydro-L-leucine; cyclopentyl-D-Gly-OH; cyclopentyl-Gly-OH; D-allylglycine; D-homocyclohexylalanine; L-1-pyrenylalanine; L-2-aminocaproic acid; L-allylglycine; L-homocyclohexylalanine; and N-(2-hydroxy-4-methoxy-Bzl)-Gly-OH.

In some instances, amino acid analogs include analogs of arginine or lysine. Examples of amino acid analogs of arginine and lysine include, but are not limited to, the following: citrulline; L-2-amino-3-guanidinopropionic acid; L-2-amino-3-ureidopropionic acid; L-citrulline; Lys(Me)₂-OH; Lys(N₃)—OH; Nδ-benzyloxycarbonyl-L-omithine; Nω-nitro-D-arginine; Nω-nitro-L-arginine; α-methyl-ornithine; 2,6-diaminoheptane dioic acid; L-ornithine; (Nδ-1-(4,4-dimethyl-dioxo-cyclohex-1-ylidene)ethyl)-D-omithine; (Nδ-1-(4,4-dimethyl-2,6-dioxo-cyclohex-1-ylidene)ethyl)-L-ornithine; (Nδ-4-methyltrityl)-D-omithine; (Nδ-4-methyltrityl)-L-ornithine; D-omithine; L-omithine; Arg(Me)(Pbf)-OH; Arg(Me)₂-OH (asymmetrical); Arg(Me)2-OH (symmetrical); Lys(ivDde)-OH; Lys(Me)2-OH·HCl; Lys(Me3)-OH chloride; Nω-nitro-D-arginine; and Nω-nitro-L-arginine.

In some instances, amino acid analogs include analogs of aspartic or glutamic acids. Examples of amino acid analogs of aspartic and glutamic acids include, but are not limited to, the following: α-methyl -D-aspartic acid; α-methyl-glutamic acid; α-methyl-L-aspartic acid; γ-methylene-glutamic acid; (N-γ-ethyl)-L-glutamine; [N-α-(4-aminobenzoyl)] -L-glutamic acid; 2,6-diaminopimelic acid; L-α-aminosuberic acid; D-2-aminoadipic acid; D-α-aminosuberic acid; α-aminopimelic acid; iminodiacetic acid; L-2-aminoadipic acid; threo-β-methyl-aspartic acid; γ-carboxy-D-glutamic acid γ,γ-di-t-butyl ester; γ-carboxy -L-glutamic acid γ,γ-di-t-butyl ester; Glu(OAll)-OH; L-Asu(OtBu)-OH; and pyroglutamic acid.

In some instances, amino acid analogs include analogs of cysteine and methionine. Examples of amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridypethyl]-DL-penicillamine, [2-(4-pyridypethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

In some instances, amino acid analogs include analogs of phenylalanine and tyrosine. Examples of amino acid analogs of phenylalanine and tyrosine include β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2;4;5-trihydroxy-phenylalanine, 3,4,5-trifluoro-D-phenylalanine, 3,4,5-trifluoro-L-phenylalanine, 3,4-dichloro -D-phenylalanine, 3,4-dichloro-L-phenylalanine, 3,4-difluoro-D-phenylalanine, 3,4-difluoro-L-phenylalanine, 3,4-dihydroxy -L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L -tyrosine, 3-methyl-D-phenylalanine, 3-methyl-L-phenylalanine, 3-nitro-D-phenylalanine, 3-nitro-L -phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

In some instances, amino acid analogs include analogs of proline. Examples of amino acid analogs of proline include, but are not limited to, 3,4-dehydro-proline, 4-fluoro-proline, cis-4-hydroxy-proline, thiazolidine-2-carboxylic acid, and trans-4-fluoro-proline.

In some instances, amino acid analogs include analogs of serine and threonine. Examples of amino acid analogs of serine and threonine include, but are not limited to, 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

In some instances, amino acid analogs include analogs of tryptophan. Examples of amino acid analogs of tryptophan include, but are not limited to, the following: α-methyl-tryptophan; β-(3-benzothienyl)-D-alanine; β-3-benzothienyl)-L-alanine; 1-methyl-tryptophan; 4-methyl-tryptophan; 5-benzyloxy-tryptophan; 5-bromo-tryptophan; 5-chloro-tryptophan; 5-fluoro-tryptophan; 5-hydroxy-tryptophan; 5-hydroxy-L-tryptophan; 5-methoxy-tryptophan; 5-methoxy-L-tryptophan; 5-methyl -tryptophan; 6-bromo-tryptophan; 6-chloro-D-tryptophan; 6-chloro-tryptophan; 6-fluoro-tryptophan; 6-methyl-tryptophan; 7-benzyloxy-tryptophan; 7-bromo-tryptophan; 7-methyl-tryptophan; D-1,2,3,4-tetrahydro-norharman-3-carboxylic acid; 6-methoxy-1,2,3,4-tetrahydronorharman-1-carboxylic acid; 7-azatryptophan; L-1,2,3,4-tetrahydro-norharman-3-carboxylic acid; 5-methoxy-2-methyl-tryptophan; and 6-chloro-L-tryptophan.

In some instances, amino acid analogs are racemic. In some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

### Cysteine-Containing Polypeptide Production

In some embodiments, a cysteine-containing polypeptide described above is generated recombinantly or is synthesized chemically. In some instances, a cysteine-containing polypeptide described above is generated recombinantly, for example, by a host cell system or in a cell-free system. In some instances, a cysteine-containing polypeptide described above is synthesized chemically.

In some embodiments, a cysteine-containing polypeptide described above is generated recombinantly by a host cell system. Exemplary host cell systems include eukaryotic cell system (e.g., mammalian cell, insect cells, yeast cells or plant cell) or a prokaryotic cell system (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyleT™ CHO-S cells, GripTite™ 293 MSR cell line, GS-CHO cell line, HepaRG™ cells, T-REx™ Jurkat cell line, Per.C6 cells, T-RExT™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprises a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable vectors for the production of a cysteine-containing polypeptide include any suitable vectors derived from either eukaryotic or prokaryotic sources. Exemplary vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of a cysteine-containing polypeptide. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

### Methods of Use

In some embodiments, disclosed herein include methods of modulating an immune response in a subject. In some embodiments, disclosed herein is a method of modulating an immune response in a subject, which comprises administering to the subject a therapeutically effective amount of a small molecule fragment of Formula (I):

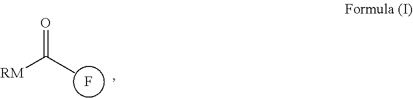

wherein:
RM is a reactive moiety selected from a Michael acceptor moiety, a leaving group moiety, or a moiety capable of forming a covalent bond with the thiol group of a cysteine residue; and
F is a small molecule fragment moiety.

In some embodiments, the small molecule fragment interacts with an endogenous cysteine-containing polypeptide expressed in the subject to form a cysteine-containing polypeptide-small molecule fragment adduct. In some instances, the cysteine-containing polypeptide-small molecule fragment adduct comprises a covalent bonding. In some cases, the cysteine-containing polypeptide-small molecule fragment adduct comprises an irreversible bonding. In other cases, the cysteine-containing polypeptide-small molecule fragment adduct comprises a reversible bonding. In some instances, an endogenous cysteine-containing polypeptide is a polypeptide that is expressed or present in a cell of interest (e.g., a diseased cell such as a cancerous cell). In some instances, an endogenous cysteine-containing polypeptide is a polypeptide that is overexpressed in a cell of interest (e.g., a diseased cell such as a cancerous cell). In some instances, an endogenous cysteine-containing polypeptide is a polypeptide that harbors one or more mutations in a cell of interest (e.g., a diseased cell such as a cancerous cell). In some instances, a mutation comprises a missense mutation, an insertion, or a deletion. In some instances, a mutation comprises a truncation, for example, a truncation at the N-terminus or the C-terminus of the polypeptide. In additional instances, an endogenous cysteine-containing polypeptide is a polypeptide that has an altered conformation in a cell of interest (e.g., a diseased cell such as a cancerous cell) relative to the conformation of the wild-type polypeptide.

In some instances, a cysteine-containing polypeptide-small molecule fragment adduct induces an immune response. In some cases, the immune response is a humoral immune response. In other cases, the immune response is a cell mediated immune response. In some instances, the cysteine-containing polypeptide-small molecule fragment adduct induces a humoral immune response. In some instances, a cysteine-containing polypeptide-small molecule fragment adduct induces a cell mediated immune response. In additional instances, a cysteine-containing polypeptide-small molecule fragment adduct induces a humoral immune response and a cell mediated immune response. In some instances, humoral immunity (or antibody-mediated beta cellularis immune system) is the production of antibody and its accessory processes such as Th2 activation, cytokine production, germinal center formation, isotype switching, affinity maturation, and memory cell generation. In some instances, humoral immunity is mediated by macromolecules in the extracellular fluids. In some cases, cell mediated immunity comprises activation of phagocytes, antigen-specific cytotoxic T-lymphocytes, and release of cytokines in response to an antigen. In some cases, cell mediated immunity differs from humoral immunity in that it does not involve production of antibody.

In some embodiments, a cysteine-containing polypeptide-small molecule fragment adduct increases an immune response relative to a control. In some cases, a cysteine-containing polypeptide-small molecule fragment adduct increases a humoral immune response relative to a control. In additional cases, a cysteine-containing polypeptide-small molecule fragment adduct increases a cell mediated immune response relative to a control. In additional cases, a cysteine-containing polypeptide-small molecule fragment adduct increases a humoral immune response and a cell mediated immune response relative to a control.

In some cases, a control is the level of an immune response in the subject prior to administration of the small molecule fragment or is the level of an immune response in a subject who has not been exposed to the small molecule fragment. In some cases, a control is the level of a humoral immune response or a cell mediated immune response in the subject prior to administration of the small molecule fragment or is the level of a humoral immune response or a cell mediated immune response in a subject who has not been exposed to the small molecule fragment.

In some instances, a cysteine-containing polypeptide-small molecule fragment adduct modulates an immune response. In some cases, the immune response is a humoral immune response. In other cases, the immune response is a cell mediated immune response. In some instances, the cysteine-containing polypeptide-small molecule fragment adduct modulates a humoral immune response. In some instances, a cysteine-containing polypeptide-small molecule fragment adduct modulates a cell mediated immune response. In additional instances, a cysteine-containing polypeptide-small molecule fragment adduct modulates a humoral immune response and a cell mediated immune response.

In some instances, a cysteine-containing polypeptide is a non-denatured form of the polypeptide.

In some instances, a cysteine-containing polypeptide comprises a biologically active cysteine site. As described above and elsewhere herein, in some cases, a biologically active cysteine site is a cysteine residue that is located about 10 Å or less to an active-site ligand or residue. In other cases, a biologically active cysteine site is a cysteine residue that is located greater than 10 Å from an active-site ligand or residue. In some cases, the cysteine residue that is located greater than 10 Å from the active-site ligand or residue is a non-active site cysteine.

Further as described elsewhere herein, a cysteine-containing polypeptide comprises, in some instances, an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide comprises an enzyme, a transporter, a receptor, a channel protein, an adaptor protein, a chaperone, a signaling protein, transcription related protein, or translation related protein.

In some embodiments, a cysteine-containing polypeptide is about 20, 30, 35, 40, 45, 50, 55, 60, 65, 70, 75, 80, 85, 90, 95, 100, 150, 200, 250, 300, 350, 400, 450, 500, 600, 700, 800, 900, 1000, 1100, 1200, 1500, 2000, 2100, 2200, 2500 amino acid residues in length or more. In some cases, a cysteine-containing polypeptide is about 20 amino acid residues in length or more. In some cases, a cysteine-containing polypeptide is about 60 amino acid residues in length or more. In some cases, a cysteine-containing polypeptide is about 70 amino acid residues in length or more. In some cases, a cysteine-containing polypeptide is about 80 amino acid residues in length or more. In some cases, a cysteine-containing polypeptide is about 90 amino acid residues in length or more. In some cases, a cysteine-containing polypeptide is about 100 amino acid residues in length or more. In some cases, a cysteine-containing polypeptide is about 150 amino acid residues in length or more. In some cases, a cysteine-containing polypeptide is about 200 amino acid residues in length or more. In some cases, a cysteine-containing polypeptide is about 300 amino acid residues in length or more. In some cases, a cysteine-containing polypeptide is about 400 amino acid residues in length or more. In some cases, a cysteine-containing polypeptide is about 500 amino acid residues in length or more. In some cases, a cysteine-containing polypeptide is about 800 amino acid residues in length or more. In some cases, a cysteine-containing polypeptide is about 1000 amino acid residues in length or more. In some cases, a cysteine-containing polypeptide is about 1500 amino acid residues in length or more.

In some embodiments, as described above, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide

In some instances, the method further comprises administration of a cysteine-containing polypeptide-small molecule fragment adduct. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples. In some cases, the cysteine-containing polypeptide-small molecule fragment adduct further enhances or increases an immune response. In some instances, an enhancement or an increase of the immune response is relative to a level of the immune response prior to administration of the cysteine-containing polypeptide-small molecule fragment adduct.

### Diseases or Indications

In some embodiments, disclosed herein include a method of administrating a small molecule fragment to a subject in which the small molecule fragment interacts with an endogenous cysteine-containing polypeptide expressed in the subject to form a cysteine-containing polypeptide-small molecule fragment adduct. In some embodiments, the cysteine-containing polypeptide is overexpressed in a disease or condition. In some cases, the overexpressed cysteine-containing polypeptide comprises one or more mutations. In some cases, the cysteine-containing polypeptide comprising one or more mutations is overexpressed in a disease or condition.

In some instances, the disease or condition is cancer. In some cases, the cysteine-containing polypeptide is a cancer-associated protein. In some cases, the cysteine-containing polypeptide is overexpressed in a cancer. In some cases, the cysteine-containing polypeptide comprising one or more mutations is overexpressed in a cancer. In some instances, a mutation comprises a missense mutation, an insertion, or a deletion. In some instances, a mutation comprises a truncation at a terminus of a protein. In some instances, a mutation alters the conformation of a protein relative to the conformation of its wild-type protein. In additional instances, a mutation does not alter the conformation of a protein.

In some instances, a cancer is a solid tumor. In some instances, a cancer is a hematologic malignancy. In some instances, a cancer is a relapsed or refractory cancer, or a metastatic cancer. In some instances, a solid tumor is a relapsed or refractory solid tumor, or a metastatic solid tumor. In some cases, a hematologic malignancy is a relapsed or refractory hematologic malignancy, or a metastatic hematologic malignancy.

In some embodiments, a cancer is a solid tumor. Exemplary solid tumor includes, but is not limited to, anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer or vulvar cancer.

In some embodiments, a cysteine-containing polypeptide described herein that is overexpressed and/or comprises one or more mutations is present in a solid tumor. In some cases, a cysteine-containing polypeptide described herein that is overexpressed and/or comprises one or more mutations is present in metastatic solid tumor. In some cases, a cysteine-containing polypeptide described herein that is overexpressed and/or comprises one or more mutations is present in a relapsed or refractory solid tumor. In some instances, a small molecule fragment described herein interacts with a cysteine-containing polypeptide that is present, overexpressed, and/or comprises a mutation in a solid tumor.

In some instances, a cancer is a hematologic malignancy. In some instances, a hematologic malignancy is a leukemia, a lymphoma, a myeloma, a non-Hodgkin's lymphoma, or a Hodgkin's lymphoma. In some instances, a hematologic malignancy comprises chronic lymphocytic leukemia (CLL), small lymphocytic lymphoma (SLL), high risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis.

In some embodiments, a cysteine-containing polypeptide described herein that is overexpressed and/or comprises one or more mutations is present in a hematologic malignancy. In some cases, a cysteine-containing polypeptide described herein that is overexpressed and/or comprises one or more mutations is present in metastatic hematologic malignancy. In some cases, a cysteine-containing polypeptide described herein that is overexpressed and/or comprises one or more mutations is present in a relapsed or refractory hematologic malignancy. In some instances, a small molecule fragment described herein interacts with a cysteine-containing polypeptide that is present, overexpressed, and/or comprises a mutation in a hematologic malignancy.

### Vaccines

In some embodiments, disclosed herein include vaccines and vaccine formulations that comprises a small molecule fragment described herein, an antibody that recognizes a cysteine-containing polypeptide-small molecule fragment adduct described herein, or a cysteine-containing polypeptide-small molecule fragment adduct described herein. In some embodiments, disclosed herein is a vaccine that comprises a small molecule fragment described herein. In some embodiments, disclosed herein is a vaccine that comprises an antibody that recognizes a cysteine-containing polypeptide-small molecule fragment adduct described herein. In some embodiments, disclosed herein is a vaccine that comprises a cysteine-containing polypeptide-small molecule fragment adduct described herein.

In some instances, a cysteine-containing polypeptide is an enzyme, a transporter, a receptor, a channel protein, an adaptor protein, a chaperone, a signaling protein, a plasma protein, transcription related protein, translation related protein, mitochondrial protein, or cytoskeleton related protein. In some cases, the cysteine-containing polypeptide is an enzyme, a transporter, a receptor, a channel protein, an adaptor protein, a chaperone, a signaling protein, transcription related protein, or translation related protein.

In some embodiments, a small molecule fragment comprises a Michael acceptor moiety which comprises an alkene or an alkyne moiety. In some instances, a covalent bond is formed between a portion of the Michael acceptor moiety on the small molecule fragment and a portion of a cysteine residue of the cysteine-containing polypeptide.

In some instances, a small molecule fragment comprises a small molecule fragment moiety F which is obtained from a compound library. In some instances, the compound library comprises ChemBridge fragment library, Pyramid Platform Fragment-Based Drug Discovery, Maybridge fragment library, FRGx from AnalytiCon, TCI-Frag from AnCoreX, Bio Building Blocks from ASINEX, BioFocus 3D from Charles River, Fragments of Life (FOL) from Emerald Bio, Enamine Fragment Library, IOTA Diverse 1500, BIONET fragments library, Life Chemicals Fragments Collection, OTAVA fragment library, Prestwick fragment library, Selcia fragment library, TimTec fragment-based library, Allium from Vitas-M Laboratory, or Zenobia fragment library.

In some instances, a small molecule fragment has a molecular weight of about 150 Dalton or higher. In some cases, a small molecular fragment has a molecular weight of about 175, 200, 225, 250, 275, 300, 350, 400, 450, 500, 550, 600, 650, 700, 750, 800, 850, 900, 950, 1000 Dalton, or higher. In some cases, the molecular weight of the small molecular fragment is prior to enrichment with a halogen, a nonmetal, or a transition metal. In some cases, the small molecular fragment of Formula (I) has a molecular weight of about 150, 175, 200, 225, 250, 275, 300, 350, 400, 450, 500, 550, 600, 650, 700, 750, 800, 850, 900, 950, 1000 Dalton, or higher.

In some instances, a vaccine is formulated in a conventional manner using one or more physiologically acceptable carriers including excipients and auxiliaries which facilitate processing of the active agents into preparations which is used pharmaceutically. Proper formulation is dependent upon the route of administration chosen. Any of the well-known techniques, carriers, and excipients are used as suitable and as understood in the art.

In some instances, a vaccine is further formulated with a cysteine-containing polypeptide-small molecule fragment adduct. In some instances, a cysteine-containing polypeptide-small molecule fragment adduct enhances an immune response. In some instances, the cysteine-containing polypeptide comprises an isolated and purified polypeptide comprising at least 80%, 85%, 90%, 95%, 96%, 97%, 98%, or 99% sequence identity to at least seven contiguous amino acids of an amino acid sequence selected from SEQs disclosed in the Examples.

In some embodiments, disclosed herein is a vaccine comprising an antibody or its binding fragment thereof that recognizes a derivative of a cysteine-containing protein having the structure of Formula (I):

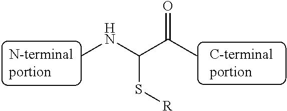

wherein,
the derivation occurs at a cysteine residue;
R is selected from:

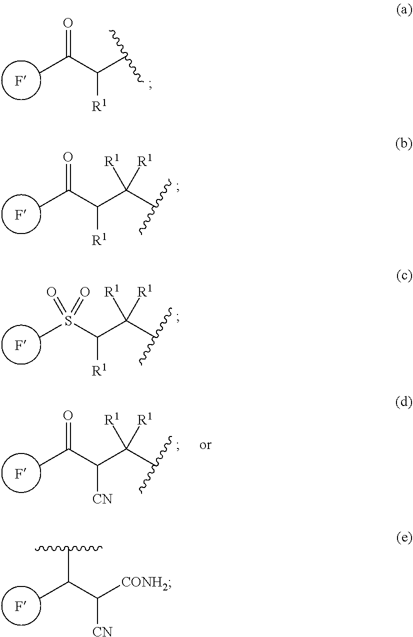

wherein,
R¹ is H, C1-C3 alkyl, or aryl; and
F′ is a small molecule fragment moiety.

In some embodiments, F′ has a molecular weight of about 175, 200, 225, 250, 275, 300, 350, 400, 450, 500, 550, 600, 650, 700, 750, 800, 850, 900, 950, 1000 Dalton, or higher. In some embodiments, the molecular weight of F′ is prior to enrichment with a halogen, a nonmetal, or a transition metal. In some embodiments, F′ is a small molecule fragment moiety illustrated in Table A.

### Pharmaceutical Compositions and Formulations

In some embodiments, disclosed herein include pharmaceutical composition and formulations comprising a small molecule fragment of Formula (I). In some instances, also described herein include pharmaceutical composition and formulations comprising a cysteine-containing polypeptide-small molecule fragment adduct. In other instances, the cysteine-containing polypeptide is an isolated and purified polypeptide comprising at least 70%, 80%, 85%, 90%, 95%, 96%, 97%, 98%, 99% or more sequence identity to at least seven contiguous amino acids of an amino acid sequence selected from SEQs disclosed in the Examples. In some embodiments, the pharmaceutical formulations described herein are administered to a subject by multiple administration routes, including but not limited to, parenteral (e.g., intravenous, subcutaneous, intramuscular), oral, intranasal, buccal, rectal, or transdermal administration routes. In some instances, the pharmaceutical composition describe herein is formulated for parenteral (e.g., intravenous, subcutaneous, intramuscular) administration. In other instances, the pharmaceutical composition describe herein is formulated for oral administration. In still other instances, the pharmaceutical composition describe herein is formulated for intranasal administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerin, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999) .

In some instances, the pharmaceutical formulations further include pH adjusting agents or buffering agents which include acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

### Therapeutic Regimens for a Pharmaceutical Composition

In some embodiments, a pharmaceutical composition described herein are administered for therapeutic applications. In some embodiments, the pharmaceutical composition is administered once per day, twice per day, three times per day or more. The pharmaceutical composition is administered daily, every day, every alternate day, five days a week, once a week, every other week, two weeks per month, three weeks per month, once a month, twice a month, three times per month, or more. The pharmaceutical composition is administered for at least 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does improve, upon the doctor's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

The foregoing ranges are merely suggestive, as the number of variables in regard to an individual treatment regime is large, and considerable excursions from these recommended values are not uncommon. Such dosages is altered depending on a number of variables, not limited to the activity of the compound used, the disease or condition to be treated, the mode of administration, the requirements of the individual subject, the severity of the disease or condition being treated, and the judgment of the practitioner.

In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)₂, Fv, single chain antibodies, diabodies, antibody chimeras, hybrid antibodies, bispecific antibodies, humanized antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular toxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface ofthe V_{H}-V_{L} dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (C_{H1}) of the heavy chain. Fab fragments differ from Fab′ fragments by the addition of a few residues at the carboxy terminus of the heavy chain C_{H1} domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. It is understood that the alkyl group is acyclic. In some instances, the alkyl group is branched or unbranched. In some instances, the alkyl group is also substituted or unsubstituted. For example, the alkyl group is substituted with one or more groups including, but not limited to, alkyl, cycloalkyl, alkoxy, amino, ether, halide, hydroxy, nitro, silyl, sulfo-oxo, or thiol. A “lower alkyl” group is an alkyl group containing from one to six (e.g., from one to four) carbon atoms. In some instances, the term alkyl group is also a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

The term “aryl” as used herein is a group that contains any carbon-based aromatic group including, but not limited to, benzene, naphthalene, phenyl, biphenyl, anthracene, and the like. The aryl group can be substituted or unsubstituted. In some instances, the aryl group is substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol. The term “biaryl” is a specific type of aryl group and is included in the definition of “aryl.” In addition, the aryl group is optionally a single ring structure or comprise multiple ring structures that are either fused ring structures or attached via one or more bridging groups such as a carbon-carbon bond. For example, biaryl refers to two aryl groups that are bound together via a fused ring structure, as in naphthalene, or are attached via one or more carbon-carbon bonds, as in biphenyl.

### EXAMPLES

These examples are provided for illustrative purposes only and not to limit the scope of the claims provided herein.

### Example 1 Synthetic Methods

### General Synthetic Methods

Chemicals and reagents were purchased from a variety of vendors, including Sigma Aldrich, Acros, Fisher, Fluka, Santa Cruz, CombiBlocks, BioBlocks, and Matrix Scientific, and were used without further purification, unless noted otherwise. Anhydrous solvents were obtained as commercially available pre-dried, oxygen-free formulations. Flash chromatography was carried out using 230-400 mesh silica gel. Preparative thin layer chromotography (PTLC) was carried out using glass backed PTLC plates 500-2000 μm thickness (Analtech). All reactions were monitored by thin layer chromatography carried out on 0.25 mm E. Merck silica gel plates (60E-254) and visualized with UV light, or by ninhydrin, ethanolic phosphomolybdic acid, iodine, p-anisaldehyde or potassium permanganate stain. NMR spectra were recorded on Varian INOVA-400, Bruker DRX-600 or Bruker DRX-500 spectrometers in the indicated solvent. Multiplicities are reported with the following abbreviations: s singlet; d doublet; t triplet; q quartet; p pentet; m multiplet; br broad. Chemical shifts were reported in ppm relative to TMS and J values were reported in Hz. Mass spectrometry data were collected on a HP1100 single-quadrupole instrument (ESI; low resolution) or an Agilent ESI-TOF instrument (HRMS).

In some embodiments, General Procedure A was used for the synthesis of one or more of the small molecule fragments and/or cysteine-reactive probes described herein. The amine was dissolved in anhydrous CH₂Cl₂ (0.2 M) and cooled to 0° C. To this, anhydrous pyridine (1.5 equiv.) was added in one portion, then chloroacetyl chloride (1.5 equiv.) dropwise and the reaction was monitored by TLC until complete disappearance of starting material and conversion to product was detected (typically 1 h). If the reaction did not proceed to completion, additional aliquots of pyridine (0.5 equiv.) and chloroacetyl chloride (0.5 equiv.) were added. The reaction was quenched with H₂O (1 mL), diluted with CH₂Cl₂ (20 mL), and washed twice with saturated NaHCO₃ (100 mL). The organic layer was concentrated in vacuo and purified by preparatory thin layer or flash column chromatography to afford the desired product. In some embodiments, General Procedure A1 is similar to General Procedure A except triethylamine (3 equiv.) was used instead of pyridine. In some embodiments, General Procedure A2 is similar to General Procedure A except N-methylmorpholine (3 equiv.) was used instead of pyridine.

In some embodiments, General Procedure B was used for the synthesis of one or more of the small molecule fragments and/or cysteine-reactive probes described herein. The amine was dissolved in anhydrous CH₂Cl₂ (0.2 M) and cooled to 0° C. To this, triethylamine (TEA, 1.5 equiv.), was added in one portion, then acryloyl chloride (1.5 equiv.) dropwise, and the reaction was monitored by TLC until complete disappearance of starting material and conversion to product was detected (typically 1 h). If the reaction did not proceed to completion, additional aliquots of TEA (0.5 equiv.) and acryloyl chloride (0.5 equiv.) were added. The reaction was quenched with H₂O (1 mL), diluted with CH₂Cl₂ (20 mL), and washed twice with saturated NaHCO₃ (100 mL). The organic layer was passed through a plug of silica, after which, the eluant was concentrated in vacuo and purified by preparatory thin layer or flash column chromatography to afford the desired product.

In some embodiments, General Procedure C was used for the synthesis of one or more of the small molecule fragments and/or cysteine-reactive probes described herein. Acryloyl chloride (80.4 μL, 1.0 mmol, 2 equiv.) was dissolved in anhydrous CH₂Cl₂ (4 mL) and cooled to 0° C. A solution of the amine (0.5 mmol, 1 equiv.) and N-methylmorpholine (0.16 mL, 1.5 mmol, 3 equiv.) in CH₂Cl₂ (2 mL) was then added dropwise. The reaction was stirred for 1 hr at 0° C. then allowed to warm up to room temperature slowly. After TLC analysis showed disappearance of starting material, or 6 h, whichever was sooner, the reaction was quenched with saturated aqueous NaHCO₃ (5 mL) and extracted with CH₂Cl₂ (3×10 mL). The combined organic layers were dried over anhydrous Na₂SO₄, concentrated in vacua, and the residue obtained was purified by preparatory thin layer chromatography to afford the desired product.

### Synthesis of Isotopically-Labeled TEV-Tags:

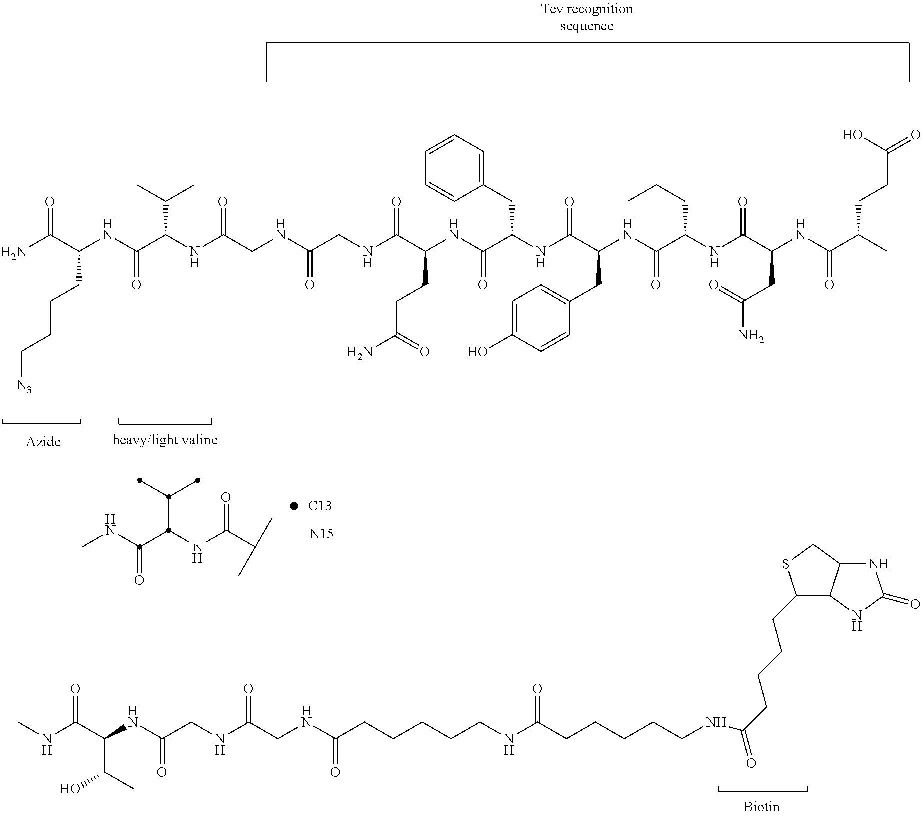

Isotopically-labeled heavy and light tags were synthesized with minor modifications to the procedure reported in Weerapana et al. Nat Protoc 2:1414-1425 (2007) and Weerapana et al. Nature 468:790-795 (2010). Fmoc-Rink-Amide-MBHA resin (EMD Biosciences; 0.5 M, 830 mg, 0.6 mmol/g loading) was deprotected with 4-methylpiperidine in DMF (50% v/v, 2×5 mL, 1 min). Fmoc-Lys(N₃)-OH (Anaspec) (500 mg, 1.26 mmol, 1.26 equiv.) was coupled to the resin overnight at room temperature with DIEA (113 μl) and 2-(6-chloro-1H-benzotriazole-1-yl)-1,1,3,3-tetramethylaminium hexafluorophosphate (HCTU; 1.3 mL of 0.5 M stock in DMF) followed by a second overnight coupling with Fmoc-Lys(N₃)-OH (500 mg, 1.26 mmol, 1.26 equiv.), DIEA (113 μ1), O-(7-azabenzotriazol-1-yl) -N,N,N′,N′-tetramethyluronium hexafluorophosphate (HATU; 1.3 mL of 0.5 M stock in DMF). Unmodified resin was then capped (2×30 min) with Ac₂O (400 μL) and DIEA (700 μL) in DMF after which the resin was washed with DMF (2×1 min). Deprotection with 4-methylpiperidine in DMF (50% v/v, 2×5 mL, 1 min) and coupling cycles (4 equiv. Fmoc-protected amino acid (EMD biosciences) in DMF) with HCTU (2 mL, 0.5 M in DMF) and DIEA (347.7 μL) were then repeated for the remaining amino acids. For the heavy TEV-tag, Fmoc-Valine-OH (¹³C₅C₁₅H₂₁¹⁵NO₄, ¹³C₅, 97-99%, ¹⁵N, 97-99%, Cambridge Isotope Laboratories, Inc.) was used. Reactions were monitored by ninhydrin stain and dual couplings were used for all steps that did not go to completion. Biotin (0.24 g, 2 equiv.) was coupled for two days at room temperature with NHS (0.1 g, 2 equiv.), DIC (0.16 g, 2 equiv.) and DIEA (0.175 g, 2 equiv.). The resin was then washed with DMF (5 mL, 2×1 min) followed by 1:1 CH₂Cl₂:MeOH (5 mL, 2×1 min), dried under a stream of nitrogen and transferred to a round-bottom flask. The peptides were cleaved for 90 minutes from the resin by treatment with 95:2.5:2.5 trifluoroacetic acid: water:triisopropylsilane. The resin was removed by filtration and the remaining solution was triturated with cold ether to provide either the light or heavy TEV-tag as a white solid. HPLC-MS revealed only minor impurities and the compounds were used without further purification. HRMS-ESI (m/z): calculated for C₈₃H₁₂₈N₂₃O₂₃S [M+H]: (Light-TEV-Tag) 1846.9268; found: 1846.9187; calculated for C₇₈¹³C₅H₁₂₈N₂₂¹⁵NO₂₃S [M+H]: (Heavy-TEV-Tag): 1852.9237; found: 1852.9309.

The synthesis of the compounds disclosed is preformed according to the general methods and procedure described in Example 1.

### Example 2

### Animal Treatment

Female DBA/1 mice (7-10 week of age) are purchased from The Jackson Laboratory (Bar Harbor, ME), and are kept for 1 week before treatments. The animal facilities are certified by the Association for Assessment and Accreditation of Laboratory Animal Care. The animals are injected i.p. with about 50 mg/kg of compound disclosed herein (dissolved in phosphate-buffered saline) or vehicle four times weekly for 3 weeks. Four days after the last dose, mice are sacrificed, and splenocytes and lymph node cells are isolated for ex vivo T-cell proliferation assays.

### Lymph Node and Splenic T-cell Proliferation Assay

Splenocytes and lymph node cells obtained from the Animal Treatment study are separately pooled from three to five mice, and single-cell suspension are prepared. The cells (about 1×10⁶ cells/well) are stimulated with 10 μg/ml of a compound disclosed herein, and then incubated for 4 days in a 96-well plate in DMEM containing 10% fetal calf serum (FCS). During the last 16 hours, the cells are pulsed with [³H]thymidine (0.5 μCi/well), and T-cell proliferation is determined by thymidine uptake. In the lymph node proliferation assay, serum-free X-VIVO medium is used.

### Electrophoresis Analysis

Splenocytes and lymph node cells obtained from the Animal Treatment study are separately pooled and centrifuged to collect the respective cell pellet. The cell pellet is subsequently lysed and resolved on a 10-12% polyacrylamide gel. Protein bands are subsequently visualized by silver staining.

### Example 3

### Tumor Cell Lines and Mice

Six to eight-week female C57BL and C3H mice are purchased (Charles River Laboratories, Wilmington, MA). The animal facilities are certified by the Association for Assessment and Accreditation of Laboratory Animal Care.

ID8 is a clone of the MOSEC ovarian carcinoma of C57BL/6 origin. SW1 is a clone derived from the K1735 melanoma of C3H origin.

### In Vivo Studies

In experiments with the ID8 ovarian carcinoma, mice (5 or 10/group) are transplanted i.p. with 3×10⁶ cells. Either 10 or 15 days later, they are injected i.p. with a compound disclosed herein or vehicle, which is repeated weekly for a total of 3 times. Mice are monitored daily for tumor growth, including swollen bellies indicating that they have developed ascites, and for evidence of toxicity. Tumor growth is recorded using a digital caliper. The survival of each mouse is further recorded and overall survival is calculated as mean±standard error of mean (M±SEM).

In experiments with the SW1 melanoma, 5×10⁵ cells are transplanted s.c. on the right flank, When the mice have developed tumors of about 4-5 mm in mean diameter, they are randomized into treatment group and control group; with either compound disclosed herein or vehicle injected i.p., respectively, at weekly intervals for a total of 3 times. Mice are monitored daily for evidence of toxicity. Tumor diameters are measured twice/week using a digital caliper and tumor surfaces are calculated. Overall survival is also recorded.

### Example 4

### Phase 1 Clinical Trial

Purpose: this clinical trial is to assess the safety and tolerability of administration of compound disclosed herein in combination with low-dose cytokines (IL-2 and IFN-alpha) in patients with metastatic or refractory cancer.
Study Type: Interventional
Study Design: Allocation: Non-Randomized
Intervention Model: Single Group Assignment
Masking: Open Label
Primary Purpose: Treatment
Primary Outcome Measures:
   Safety [Time Frame: Initial dose of study therapy through 30 days post last dose of study therapy]
   Tolerability [Time Frame: Initial dose of study therapy through 30 days post last dose of study therapy]
   Anti-tumor Activity [Time Frame: From initial dose of study therapy to disease progression]

### Eligibility

Ages Eligible for Study: 18 Years and older (Adult, Senior)
Sexes Eligible for Study: All
Accepts Healthy Volunteers: No

### Criteria

### Inclusion Criteria:

Have a histologically confirmed diagnosis of metastatic or refractory cancer for which there are no effective standard therapeutic options available;
Have signed an Institutional Review Board (IRB) approved informed consent form (ICF) prior to performing any study evaluation/procedures;
Be >or =18 years of age and women must either be 1) not of childbearing potential or 2) have a negative serum pregnancy test within 7 days prior to commencing treatment. Patients are considered not of childbearing potential if they are surgically sterile (they have undergone a hysterectomy, bilateral tubal ligation or bilateral oophorectomy) or they are postmenopausal (12 consecutive months of amenorrhea [lack of menstruation]);
(If applicable) Have completed prior cytotoxic chemotherapy, radiotherapy or immunotherapy or experimental therapy >or =30 days prior to the study enrollment, and recovered form associated toxicities;
Have an Eastern Cooperative Oncology Group (ECOG) score of <or =2, and an anticipated life expectancy of at least 6 months;
Have adequate hematologic function, as defined by an absolute or calculated neutrophil count >or =1500/microL, platelet count >or =100000/microL, lymphocyte count >or =500/microL, and hemoglobin level >or =10 g/dL. Patients may not receive prophylactic transfusion in order to qualify for trial eligibility;
Have adequate renal function, as defined by a documented serum creatinine of <or =2.0 mg/dL. Greater than “1+” proteinuria will require microscope evaluation and the results discussed with the medical monitor prior to patient enrollment; or if serum creatine is >2.0, patient must have an actual or calculated 24-hour creatinine clearance of >60 mL/min and no obvious evidence of concurrent medullary cystic disease or obstructive uropathy;
Have adequate hepatic function, as defined by a total bilirubin level <or =1.5×upper limit of normal (ULN) and alkaline phosphatase, aspartate transaminase (AST), and alanine transaminase (ALT) levels <or =2.5×ULN. If alkaline phosphatase is outside of these parameters and is due to bone metastases (as verified by the assessment of isoenzymes), then the patient is eligible.

### Exclusion Criteria:

Have a history of severe hypersensitivity (grade 3-4 allergic reaction) to fluorescein or any drug, radiologic contrast agent, insect bite, food, cytokines, or any other agent; or have received fluorescein within 30 days of the study;
Have medical conditions that preclude the use of IL-2 or IFN-alpha. These conditions include but are not limited to, diabetes mellitus with a history of progression to diabetic ketoacidosis, history of severe coagulation disorder, psoriasis, sarcoidosis, retinal hemorrhage, symptomatic pulmonary disease, heart failure (>or =New York Heart Association NYHA class II), or transplant requiring immunosuppressive therapy;
Be pregnant or breast-feeding;
Be currently receiving an experimental drug, or used an experimental device within 30 days of study entry;
Be currently undergoing chemotherapy, anticancer hormonal therapy, and/or therapy with immuno suppressant agents;
Have any concomitant malignancy with the exception of basal cell or squamous cell carcinoma of skin;
Have radiographically documented evidence of current brain metastases, a history of stem cell transplant, immunodeficiency, and/or a medical or psychiatric illness (that in the investigator's opinion, would prevent adequate compliance with study therapy or evaluation of the endpoints).

### Example 5: General Methods

Isolation of Peripheral Blood Mononuclear Cells (PBMC) and T cells

Peripheral blood mononuclear cells (PBMCs) are isolated over Lymphoprep (STEMCELL Technologies) gradient using slightly modified manufacturer's instructions. 25 mL of freshly isolated blood is layered on top of 12.5 mL of Lymphoprep in a 50 mL Falcon tube minimizing mixing of blood with Lymphoprep. The tubes are centrifuged at room temperature (20 min, 23° C.) with break off and the plasma and Lymphoprep layers containing PBMCs are transferred to new 50 mL Falcon tubes with a 2:1 dilution with PBS. The cells are pelleted (8 min, 4° C.) and washed with PBS once. T cells are isolated from fresh PBMCs using EasySep Human T Cell Isolation Kit (STEMCELL Technologies, negative selection) according to manufacturer's instructions.

### T Cell Activation for Mass-Spectrometry Analysis

A non-tissue culture treated 6-well plate are pre-coated with αCD3 (1.5 μg/mL) antibody in PBS (3 mL/well) and kept at 4° C. overnight. The next day, the plates are transferred to a 37° C. incubator for 1 h and washed with PBS (2×5 mL/well). Freshly isolated T cells are re-suspended in RPMI media (10% FBS, L-glutamine (2 mM), penicillin (100 U/mL), streptomycin (100 μg/mL)), containing αCD28 antibody (1 μg/mL) at 1×10⁶ cells/mL, plated into the pre-coated 6-well plate (6-10 mL/well) and kept at 37° C. in a 5% CO₂ incubator for 3 days. Following this incubation period, the cells are combined in 50 mL Falcon tubes, pelleted (5 min, 4° C.), and washed with PBS (10 mL). The cells are then re-suspended in RPMI media containing recombinant IL2 (10 U/mL) and kept at 37° C. in a 5% CO₂ incubator for 10-12 days, splitting the cells every 3-4 days to keep cell density below 2×10⁶ cells/mL. After this time, the cells are pelleted (5 min, 4° C.), washed with PBS (10 mL) and either re-suspended in fresh RPMI media for in situ treatments or flash-frozen and kept at −80° C. until further analysis (in vitro treatments).

### In Situ Labeling With Cysteine-Reactive Electrophiles

Activated or expanded T cells are re-suspended in RPMI media supplemented with 10% FBS, L-glutamine (2 mM), penicillin (100 U/mL), and streptomycin (100 μg/mL) at 2×10⁶ cells/mL. The compounds are added to cells as 1000× DMSO stocks and mixed well with the media by pipetting up and down after addition. The cells are kept at 37° C. in 5% CO₂ containing incubators for 3 h, then pelleted by centrifugation (5 min, 4° C.), washed with cold PBS (10 mL) and transferred to Eppendorf tubes (1 mL PBS). The cells are pelleted again (5 min, 4° C.), flash-frozen, and kept at −80° C. until further analysis.

### Multidimensional Screen for Inhibition of T Cell Activation

Non-tissue culture treated 96-well plates are pre-coated with αCD3 (5 μg/mL) and αCD28 antibodies (2 μg/mL) in PBS (100 μL/well) and left at 4° C. overnight. Freshly isolated T cells are re-suspended in RPMI media supplemented with 10% FBS, L-glutamine (2 mM), penicillin (100 U/mL), and streptomycin (100 μg/mL) at 2×10⁶ cells/mL. Compound stocks (200×) in DMSO are diluted to 2× stocks in the working RPMI media in another 96-well plate. The pre-coated 96-well treatment plates are washed with PBS (2×200μL), T cells (100 μL/well, 2×10⁵ cells/well) are then added to the wells, followed by the addition of 2× compound stocks in RPMI media (100 μL). The treatment is done overnight (24 h) at 37° C. in a 5% CO₂ containing incubator. Following the treatment, the cells are transferred to a U-bottom 96-well plate and harvested by centrifugation (3 min, 4° C.). The supernatants are kept and stored at −80° C. for further cytokine analysis, while the cells were washed with PBS (2×150 μL) prior to staining for flow cytometry analysis.

### Flow Cytometry Analysis

Following the PBS washes, the cells are stained with fixable near-IR LIVE/DEAD cell stain (Invitrogen) according to manufacturer's instructions. Briefly, one vial of near-IR LIVE/DEAD dye is resuspended in DMSO (50 μL) and diluted with PBS (1:1000). The diluted stain is added to each well (200 iuL) and the cells are incubated for 30 min at room temperature in the dark. After this time, the cells were pelleted (3 min, 4° C.), washed once with PBS (200 μL/well) and incubated with a freshly made cocktail of antibodies for the appropriate cell surface markers diluted in PBS containing 2% FBS (1:400 antibody dilution).

### Measurement of Phospho-NF-κB p65 (Ser536) Levels

Freshly isolated T cells (2×10⁵ cells/well) are harvested and stimulated as described before in a 96-well plate in the presence of DMSO or compounds of interest. Following the overnight treatment, the cells are pelleted in a U-bottom plate, harvested by centrifugation (3 mM, 4° C.), washed with PBS, and stained with near-IR LIVE/DEAD dye. After the staining, intracellular phospho-NF-κB p65 (Ser536) levels is measured using PE conjugate of phospho-NF-κB p65 (Ser536) (93H1) rabbit antibody (Cell Signaling Technology) according to manufacturer's instructions. The cells are washed with PBS and fixed with 4% PFA in PBS (100 μL, 15 min, rt). The cells are washed with PBS again (2×150 μL), placed on ice and permeabilized with 90% MeOH (100 μL/well, slow addition with gentle mixing by pipetting up and down). Following a 30 min incubation on ice, the plate is sealed and stored at −20° C. overnight. The following day, the cells are thawed on ice, washed with PBS (150 μL×2), and stained with PE conjugate of phospho-NF-κB p65 (Ser536) (93H1) rabbit antibody (50 μL, 1:100 dilution in incubation buffer (1% FBS in PBS)) for 1 h at it in the dark. The cells are then washed with incubation buffer (150 μL×2) and resuspended in PBS for further flow cytometry analysis.

### Measurement of Intracellular Glutathione Levels

Intracellular glutathione levels were determined using GSH-Glo glutathione assay (Promega Corporation) according to manufacturer's instructions. Briefly, freshly isolated T cells are treated with compounds or DMSO overnight under TCR-stimulating conditions (96-well plate, 1×10⁵ cells/well) at 37° C. in 5% CO₂ containing incubator, then transferred to a U-shape bottom 96-well plate and pelleted (3 min, 4° C.). The supernatants are kept and stored at −80° C. for cytokine analysis. The cells are washed with PBS (2×150 μL) and re-suspended in 50 μL of PBS. An aliquot of treated cells (25 μL) is then added to an equal volume of 2× GSH reaction buffer containing Glutathione S-transferase and Luciferin-NT substrate (1:50 dilution in GSH-Glo Reaction Buffer). The reaction is incubated for 30 min at rt, after which Luciferin Detection Reagent (in reconstitution buffer with esterase, 25 μL/well) is added, and the plate is incubated for an additional 15 mM and luminescence is read using a CLARIOstar (BMG Labtech) plate reader.

### DuoSet ELISA Quantification of Secreted Cytokines (IL2, IFNγ, TNFα)

The levels of secreted IL2, IFNγ and TNFα after incubating T cells in the presence of DMSO or electrophilic compounds under TCR-stimulating conditions are measured using DuoSet ELISA cytokine kits (R&D Systems) in clear microplates (R&D Systems) according to manufacturer's instructions and read using a CLARIOstar (BMG Labtech) plate reader (450 nm).

NFAT (nuclear Factor of Activated T Cells) Luciferase Reporter Assay

NFAT activity are measured using the Jurkat-Lucia NFAT reporter cell line (Invivogen) according to manufacturer's procedure. Briefly, Jurkat-Lucia NFAT cells are cultured at 37° C. in 5% CO₂ containing incubator in manufacturer-recommended growth medium (RPMI, 2 mM L-glutamine, 25 mM HEPES, 10% heat-inactivated fetal bovine serum (FBS, 30 min at 56° C.), 100 μg/mL Normocin, Pen-Strep (50 U/mL-50iAg/mL)) keeping cell density below 2×10⁶ cells/mL. Zeocin (100 μg/mL) is added to the growth medium every other passage and the cell passage number is kept less than 10. On the day of compound treatment, the cells are pelleted and resuspended at 2.2×10⁶ cells/mL in fresh, pre-warmed test medium (RPMI, 2 mM L-glutamine, 25 mM HEPES, 10% heat-inactivated FBS, Pen-Strep (100 U/mL-100 μg/mL) without Normocin). Cell suspension (180 μL, 4×10⁵ cells/well) is then added to the test plate containing stimulating solution (20 μL/well, PMA (50 ng/mL) and ionomycin (3 Kg/mL) in growth media) and test compounds (2 μL, 100× stock in DMSO) or DMSO, and the plate is kept at 37° C. in a 5% CO₂ containing incubator for 24 h. To evaluate expression of the luciferase reporter, 50 μL of Quanti-luc (Invivogen) detection reagent is combined with 20 μL of cell suspension from each well in a new 96-well white (opaque) plate and the luminescence was read using a CLARIOstar microplate reader (BMG Labtech).

### Bio-Plex Quantification of Secreted Cytokines

Freshly isolated PBMCs (4×10\hat{ }6 cells/mL, 1 mL/well), were treated with compound (10 μM) or vehicle (DMSO) for 6 h in a 24-well plate, after which cGAMP (10 μM) is added to the wells and the cells were incubated for additional 20 h. Following this treatment, the cells are transferred to 1.5 mL Eppendorf tubes and harvested by centrifugation (8 min, 4° C.). The supernatants are saved (−80° C.) and used for further cytokine analysis using Bio-Plex Pro Human Cytokine assay (Bio-Rad) according to manufacturer's instructions. Bio-Plex Assay is a multiplex flow immunoassay that simultaneously detects and identifies cytokines based on fluorescent dye-labeled 6.5 μm magnetic beads in a single reaction.

### ELISA Quantification of Secreted IFN-β

Concentrations of IFN-β are determined with VeriKine-HS human IFN-β serum ELISA kit (PBL Assay Science) according to manufacturer's instructions. All concentrations of IFN-β are calculated according to the standard curve generated for each experiment.

### Western Blot Analysis

Western blot analysis is performed on freshly isolated or expanded T cells. For Western blot protein degradation analysis, primary human T cells (2×10⁷ cells/treatment) are re-suspended in RPMI media at 2×10⁶ cells/mL and treated with the compounds or DMSO at 37° C. in a CO₂ containing incubator for 24 h. Following this incubation period, the cells are pelleted (5 min, 4° C.), washed with PBS (10 mL), transferred to 1.5 mL Eppendorf tubes and flash-frozen until further analysis. On the day of the analysis, the cell pellets are thawed on ice, re-suspended in cold PBS and lysed by sonication with probe sonicator (2×8 pulses). Protein concentrations for all the samples are adjusted to 1 mg/mL, 4× loading buffer was added (10 μL to 30 μL of proteome), and the samples are heated at 95° C. for 5 min. The proteins are resolved using SDS-PAGE (10% acrylamide gel) and transferred to 0.45 μM nitrocellulose membranes (GE Healthcare). The membrane is blocked with 5% milk in Tris-buffered saline with tween (TBST) buffer (0.1% Tween 20, 20 mM Tris-HCl7.6, 150 mM NaCl) at rt for 1 h (or at 4° C. overnight), washed 3 times with TBST, and incubated with primary antibodies in 5% BSA in TBST at 4° C. overnight. Following another TBST wash (3 times), the membrane is incubated with secondary antibody (1:5,000 in 5% milk in TBST) at 4° C. overnight. The membrane is washed with TBST (3 times), developed with ECL western blotting detection reagent kit (Thermo Scientific) and recorded on CL-X Posure film (Thermo Scientific). Relative band intensities are quantified using ImageJ software. (Rasband, W. S., ImageJ, U.S. National Institutes of Health, Bethesda, Maryland, USA, https://imagej.nih.gov/ij/, 1997-2018.)

### Western Blot Analysis of Chromatin-Bound Proteins

Compound (10 μM) treated expanded T cells are washed with PBS before permeabilization by rotation at 4° C. for 10 min with cytoplasm lysis buffer (10 mM sodium phosphate pH 7.4, 25 mM KC1, 1.5 mM MgCl₂, 10% glycerol, and 0.025% NP40 supplemented with 1× HALT protease inhibitor cocktail (Thermo Scientific)). Nuclei are pelleted and washed with cytoplasm lysis buffer without detergent, before being lysed by gentle sonication (Branson Sonifier 250) in cell lysis buffer (10 mM sodium phosphate pH 7.4, 25 mM KCl, 1.5 mM MgCl₂, 10% glycerol, and 1% NP40, 0.1% SDS supplemented with 1× HALT, and 1× Benzoase (Pierce)) and rotated for 2 h at 4° C. Insoluble material is precipitated by centrifugation (12,000 g, 10 min) and the protein concentration of nuclear extracts is measured using standard BCA assay (Thermo Scientific) and normalized. Electrophoretic separation was performed on Novex 4-20% Tris-Glycine Mini Gels (Invitrogen) using the Novex Wedgewell system, and transferred to 0.45 μM Nitrocellulose membranes (GE Healthcare). Primary antibodies are applied overnight at 4° C. in 5% BSA/TBST. Blots are imaged using fluorescence-labeled secondary antibodies (LI-COR) on the Odyssey CLx Imager.

Gene Expression (qPCR) Analysis

Total RNA from compound or DMSO treated T cells (1.5×10⁷ cells/group) is isolated using RNeasy Mini Kit (Qiagen) according to manufacturer's protocol. RNA concentration is determined using NanoDrop and adjusted to 1 μg RNA in 15 μL RNAse free water for the reverse transcription reaction. cDNA amplification was done using iScript Reverse Transcription Supermix kit (BioRad) according to manufacturer's instructions. The following PCR settings are used for the reverse transcription reaction: 5 min at 25° C. (priming), 20 min at 46° C. (Reverse transcription), 1 min at 95° C. (RT inactivation), hold at 4° C. qPCR analysis are performed on ABI Real Time PCR System (Applied Biosystems) with the SYBR green Mastermix (Applied Biosystems). Relative gene expression are normalized to actin.

qPCR primers used (5′ to 3′):

actin-fwd AGAGCTACGAGCTGCCTGAC
actin-rev AGCACTGTGTTGGCGTACAG
BIRC2-fwd AGCACGATCTTGTCAGATTGG
BIRC2-rev GGCGGGGAAAGTTGAATATGTA
BIRC3-fwd AAGCTACCTCTCAGCCTACTTT
BIRC3-rev CCACTGTTTTCTGTACCCGGA
IL6-fwd AATTCGGTACATCCTCGACGG
IL6-rev GGTTGTTTTCTGCCAGTGCC
IL 1-beta-fwd ACAGATGAAGTGCTCCTTCCA
IL 1-beta-rev GTCGGAGATTCGTAGCTGGAT
CXCL10-fwd CCAGAATCGAAGGCCATCAA
CXCL10-rev CATTTCCTTGCTAACTGCTTTCAG

### RNA Sequencing

Total RNA from compound or DMSO treated T cells (1.5×10⁷ cells/group) are isolated using RNeasy Mini Kit (Qiagen) using RNAse free DNAse set (Qiagen) for on column DNA digestion according to manufacturer's protocol and stored at −80° C. until further analysis.

RNA quality is assessed using Tape Station 4200 and RNA-Seq libraries are prepared using the TruSeq stranded mRNA Sample Preparation Kit v2 according to Illumina protocols. Multiplexed libraries were validated using TapeStation 4200, normalized, pooled and quantified by qPCR for sequencing. High-throughput sequencing is performed on the NextSeq 500 system (Illumina). Image analysis and base calling is done with Illumina CASAVA-1.8.2.

Sequenced reads are quality-tested using FASTQC Andrews S. (2010). FastQC: a quality control tool for high throughput sequence data. Mapping is carried out using default parameters (up to 10 mismatches per read, and up to 9 multi-mapping locations per read). The genome index is constructed using the gene annotation supplied with the hg19 Illumina iGenomes (iGenomes online. Illumina. 2015. http://support.illumina.com/sequencing/sequencing_software/igenome.html) collection and sjdbOverhang value of 100. Homer (Heinz et al., 2010) v4.10.4 was used to calculate the fragments per kilobase per million mapped reads (FPKM) normalized gene expression across all exons with the top expressed isoform reported.

### Example 6 General ABPP Preparation and Procedures

isoTOPABPP Sample Preparation

Activated or expanded primary human T cells were re-suspended in RPMI (1×10⁶ cells/mL) containing 10% fetal bovine serum (FBS), penicillin (100 U/mL), streptomycin (100 mg/mL), and L-glutamine (2 mM). The cells were treated with DMSO or compounds for 3 h, pelleted (524 g, 5 min), washed with PBS, and lysed by sonication (2×8 pulses). Soluble and particulate proteomic fractions were separated by ultracentrifugation (100,000 g, 45 min), and protein concentration was normalized to 1.7 mg/mL using a standard DC protein assay (Bio-Rad). The resulting proteomes were analyzed by competitive isotopic Tandem Orthogonal Proteolysis Activity-Based Protein Profiling (isoTOP-ABPP).

### IA-Alkyne Labeling and Click Chemistry

Samples (500 μL, 1.7 mg/mL) were treated with iodoacetamide alkyne (IA-alkyne, 5 μL of 10 mM stock in DMSO, final concentration: 100 μM) for 1 hat ambient temperature. Modified proteins were then conjugated to isotopically labeled, TEV-cleavable biotin tags (TEV-tags) using copper-catalyzed azide-alkyne cycloaddition reaction (CuAAC). Reagents for the CuAAC reaction were pre-mixed prior to their addition to the proteome samples. TEV tags (light or heavy, 10 μL of 5 mM stocks in DMSO, final concentration=100 μM), tris(benzyltriazolylmethyl)amine ligand (TBTA; 30 μL of 1.7 mM stock in DMSO:t-butanol 1:4, final concentration=100 μM), tris(2-carboxyethyl)phosphine hydrochloride (TCEP; 10 μL of fresh 50 mM stock in water, final concentration=1 mM), and Cu(OAc)₂ (10 μL of 50 mM stock in water, final concentration=1 mM) were combined in an Eppendorf tube, vortexed and added to the proteomes (55 μL/sample). “Heavy” CuAAC reaction mixture was added to the DMSO-treated control samples and “light”—to compound-treated samples. The reaction was allowed to proceed at rt for 1 h, “heavy” and “light” samples were combined pairwise in 15 mL conical Falcon tubes kept on ice containing 4 mL of cold methanol (pre-chilled at −80° C.), 1 mL CHCl₃, and 1 mL H₂O. Eppendorf tubes from the reaction mixtures were washed with additional H₂O (1mL each) and the washes were added to the same Falcon tube (final ratios MeOH:CHCl₃:H₂O=4:1:4). Following centrifugation (5,000 g, 10 min, 4° C.), a protein disk formed at the interface of CHCl₃ and aqueous layers. Both layers were aspirated without perturbing the disk, which was resuspended in cold MeOH (2 mL) and CHCl₃ (1 mL) by vortexing. The proteins were pelleted (5,000 g, 10 min, 4° C.), and the resulting pellets were solubilized in 1.2% SDS in PBS (1 mL) with sonication and heating (95° C., 5 min).

isoTOPABPP Sample Streptavidin Enrichment

Once solubilized, the samples were diluted with PBS (4 mL) and streptavidin-agarose beads were added for the enrichment (final SDS concentration: 0.2% in PBS). The beads (100 μL of a 50% slurry per sample) were washed with PBS (2×10 mL) and resuspended in 1 mL of PBS per sample prior to addition. The final mixture was rotated for 3 h at rt. Following this enrichment step, the beads were pelleted by centrifugation (2,000 g, 2 min) and extensively washed to remove non-specifically binding proteins (2×10 mL 0.2% SDS in PBS, 2×10 mL PBS, and 2×10 mL _{H2O}).

isoTOPABPP Sample Trypsin and TEV Digestion

After the last wash, the beads were transferred to new Eppendorf tubes in water (2×0.5 mL), pelleted (4,000 g, 3 min), and resuspended in 6M urea in PBS (0.5 mL). DTT (25 μL of a fresh 200 mM stock in water, final concentration—10 mM) was added and the beads were incubated at 65° C. for 15 min. Iodoacetamide (25 μL of a 400 mM stock in water, final concentration—20 mM) was then added and the samples were incubated in the dark at 37° C. with shaking for 30 min. Following this incubation, the mixture was diluted with PBS (900 μL), the beads were pelleted by centrifugation and resuspended in 2M urea in PBS (200 μL). Trypsin (Promega, sequencing grade; 2 μg in 6 μL of trypsin buffer containing 1 mM CaCl₂) was added to the mixture and the digestion was allowed to proceed overnight at 37° C. with shaking. The beads were pelleted (2,000 g, 2 min) and the tryptic digest was aspirated. The beads were then extensively washed (3×1 mL PBS, 3×1 mL H₂O), transferred to a new Eppendorf tube in H₂O (2×0.5 mL), washed with TEV buffer (200 μL, 50 mM Tris, pH 8, 0.5 mM EDTA, 1 mM DTT), and resuspended in TEV buffer (140 μL). TEV protease (4 μL, 80 μM) was then added and the beads were incubated at 30° C. overnight with rotation. Following the overnight digestion, the beads were pelleted by centrifugation (2,000 g, 2 min) and the TEV digest was separated from the beads using Micro Bio-Spin columns (Bio-Rad) with centrifugation (800 g, 0.5 min) and an additional wash (100 μL H₂O). The samples were then acidified by the addition of 0.1% FA (14 μL, final concentration: 5% v/v) and stored at −80° C. prior to analysis.

isoTOP ABPP Liquid-Chromatography-Mass-Spectrometry (LC-MS/MS) Analysis

Samples were pressure-loaded onto a 250 μm (inner diameter) fused silica capillary columns packed with C18 resin (Aqua 5 μm, Phenomenex) and analyzed by multidimensional liquid chromatography tandem mass-spectrometry (MudPIT) using an LTQ-Velos Orbitrap mass spectrometer (Thermo Scientific) coupled to an Agilent 1200-series quaternary pump. The peptides were eluted onto a biphasic column with a 5 μm tip (100 μm fused silica, packed with C18 (10 cm) and bulk strong cation exchange resin (3 cm, SCX, Phenomenex) in a 5-step MudPIT experiment, using 0%, 30%, 60%, 90%, and 100% salt bumps of 500 mM aqueous ammonium acetate and a 5%-100% gradient of buffer B in buffer A (buffer A: 95% water, 5% CH₃CN, 0.1% FA; buffer B: 5% water, 95% CH₃CN, 0.1% FA). Data were collected in data-dependent acquisition mode with dynamic exclusion enabled (20 s, repeat of 2). One full MS (MS1) scan (400-1800 m/z) was followed by 30 MS2 scans (ITMS) of the nth most abundant ions.

isoTOP ABPP Peptide Identification

The MS2 spectra data were extracted from the raw file using RAW Converter (version 1.1.0.22; available at hap://fields.scripps.edu/rawconv/), uploaded to Integrated Proteomics Pipeline (IP2), and searched using the ProLuCID algorithm (publicly available at http://fields.scripps.edu/downloads.php) using a reverse concatenated, non-redundant variant of the Human UniProt database (release-2012_11). Cysteine residues were searched with a static modification for carboxyamidomethylation (+57.02146) and up to one differential modification for either the light or heavy TEV tags (+464.28595 or +470.29976 respectively). Peptides were required to have at least one tryptic terminus and to contain the TEV modification. ProLuCID data was filtered through DTASelect (version 2.0) to achieve a peptide false-positive rate below 1%.

isoTOP ABPP R Value Calculation and Data Processing

The heavy/light isoTOP-ABPP ratios (R values) for each unique peptide (DMSO/compound treated) were quantified with in-house CIMAGE software (Weerapana et al., 2010) using default parameters (3 MS1 acquisitions per peak and signal to noise threshold set to 2.5). Site-specific engagement of cysteine residues was assessed by blockade of IA-alkyne probe labeling. A maximal ratio of 20 was assigned for peptides that showed a ≥95% reduction in MS1 peak area in the compound treated proteome (light TEV tag) compared to the control DMSO-treated proteome (heavy TEV tag). Ratios for unique peptide sequences were calculated for each experiment; overlapping peptides with the same modified cysteine (e.g., different charge states, elution times or tryptic termini) were grouped together and the median ratio was reported as the final ratio (R). Additionally, ratios for peptide sequences containing multiple cysteines were grouped together. When aggregating data across experimental replicates, the mean of each experimental median R was reported. The peptide ratios reported by CIMAGE were further filtered to ensure the removal or correction of low-quality ratios in each individual dataset. The quality filters applied were the following: removal of half-tryptic peptides, removal of peptides with more than one tryptic miscleaved, removal of peptides with R=20 and only a single MS2 event triggered during the elution of the parent ion, removal of non-unique peptides. Further filtering was then performed as described below for each experiment type.

### Combining Data Across Experimental Groups

Identifiers consisting of the Uniprot accession concatenated with the tryptic sequence associated with the particular peptide were used. Peptides that contained the same modified cysteine or where multiple cysteines were modified on that peptide were combined. When data from an experiment group associated with a miscleaved peptide sequence was combined with data from another group which contained a non miscleaved variant of the same peptide, all data was reported under the fully tryptic identifier, unless the non miscleaved variant introduced an additional cysteine, in which case the data was not merged.

### Filtering of Broad Ligandability (Scout Fragment) Data

All peptides with R=20 were manually reviewed. Peptides with R=20 were discarded if the ratio set contained a single 20, and the minimum ratio in the set was less than 4. If the ratio set contained two or more 20 values and the minimum ratio in the set was less than 2, these 20 values were also discarded. This filter was applied on R values within a single experiment and when aggregating data from replicate experiments.

When aggregating data from replicate experiments, for peptides that had standard deviations greater than 60% of the mean, the lowest ratio of that set was reported, unless the minimum ratio of the set was ≥4, in which case the average ratio was reported. Individual peptide sequences were required to have been quantified (R≠0) in at least two replicates per condition. Peptides were considered liganded if they had a final value of R≥5.

### Filtering of Elaborated Compound Data

All peptides with R=20 were manually reviewed. Within individual replicates, peptides with R=20 were discarded if the ratio set contained a single 20, and the minimum ratio in the set was less than 4. When aggregating ratios across replicates, peptides with R=20 were discarded if the ratio set contained a single 20, and the minimum ratio in the set was less than 3. Individual peptide sequences were required to have been quantified (R≠0) in at least two replicates per condition, unless they had R≥4 in both particulate and soluble conditions for a given compound.

During manual review of the data, some peptides were exempted from specific filters due to additional evidence of their validity. BIRC2 (C45) R=20 and CASP2 (C366, C370) R=20 values derived from DMF datasets values derived from DMF datasets were exempted from applied 20-filters as the same residues were convincingly liganded in the TMT datasets.

Peptides were considered liganded if they had a final value of R≥4.

### Filtering and Processing of Hyper-Reactivity Data

Peptides with R=20 were discarded if the ratio set contained a single 20, and the minimum ratio in the set was less than 4 This filter was applied on R values within a single experiment and when aggregating data from replicate experiments.

Data from these experiments was separated according to activation state and the minimal ratio between soluble and particulate fractions for each state was reported for each peptide.

### TMT-ABPP Sample Preparation and IA-D TB Labeling

Samples (500 μL, 1.7 mg/mL) were treated with iodoacetamide desthiobiotin (IA-DTB, 5 μL of 10 mM stock in DMSO, final concentration: 100 μM) for 1 h at ambient temperature. Ice-cold MeOH (500 μL) and CHCl₃ (200 μL) were then added, the mixture was vortexed and centrifuged (10,000 g, 10 min, 4° C.) to afford a protein disc at the interface of CHCl₃ and aqueous layers. Both layers were aspirated without perturbing the disk, which was re-suspended in cold methanol (500 μL) and CHCl₃ (200 μL) by sonication. The proteins were pelleted (10,000 g, 10 min, 4° C.), and the resulting pellets were re-suspended in a buffer (90 μL) containing 9M urea, 10 mM DTT and 50 mM triethylammonium bicarbonate ( 1/20 dilution of 1.0 M stock solution, pH 8.5) by thorough pipetting up and down. The resulting mixture was heated at 65° C. for 20 min. Sample was cooled to room temp, iodoacetamide (10 μL, 500 mM solution in H₂O) was added, and the samples were incubated at 37° C. for 30 min with shaking.

### TMT-ABPP Trypsin Digestion and Streptavidin Enrichment

Following the labeling with iodoacetamide, samples were diluted with 305 μL of triethylammonium bicarbonate buffer (50 mM, 1/20 dilution of 1.0 M stock, pH 8.5; Final urea concentration: 2.0 M). Trypsin (4 μL of 0.25 μg/μL trypsin in trypsin buffer, containing 25 mM CaCl₂) was then added and the proteins were digested at 37° C. overnight. The following day, samples were diluted with wash buffer (400 μL, 25 mM Tris-HCl, pH 7.5, 150 mM NaCl, 0.2% NP-40), streptavidin-agarose beads (50% slurry in wash buffer) were added to each sample (40 μL/sample) and the bead mixture was rotated for 2 h at rt. Briefly, for a 10-plex sample, streptavidin-agarose bead slurry (440 μL, 50% slurry) was washed (2×1 mL, 25 mM Tris-HCl, pH 7.5, 150 mM NaCl, 0.1% NP-40) and brought up to the initial volume in the wash buffer prior to the addition to the sample. After incubation, the beads were pelleted by centrifugation (2,000 g, 1 min), transferred to a BioSpin column and washed extensively (3×1 mL wash buffer, 3×1 mL PBS, 3×1 mL H₂O). Peptides were eluted by the addition of 300 μL of 50% aqueous CH3CN containing 0.1% FA. The eluate was then evaporated to dryness using SpeedVac vacuum concentrator.

TMT Tag labeling

Peptides were resuspended in 100 μL EPPS buffer (200 mM, pH 8.0) with 30% dry CH3CN, vortexed and spun down (2,000 g, 1 mM). TMT tags (3 μL/tube in dry CH₃CN, 20 μg/μL) were added to the corresponding tubes and the reaction was allowed to proceed for 1 h 15 min. The reaction was quenched by the addition of 5% hydroxylamine (3 iAL per sample), vortexed and left at room temperature for 15 min. FA (5 μL) was then added to each tube, the tubes were vortexed, spun down and combined in a low binding 1.5 mL Eppendorf tube. The final combined sample was dried in a SpeedVac vacuum concentrator and kept at −80° C. until the high pH fractionation step.

### High pH Fractionation

The spin columns for high pH fractionation were pre-equilibrated prior to use. Briefly, the columns were placed in Eppendorf tubes (2 mL), spun down to remove the storage solution (5,000 g, 2 min), and washed with CH₃CN (2×300 μL, 5,000 g, 2 min) and buffer A (2×300 μL, 95% H₂O, 5% CH₃CN, 0.1% FA, 5,000 g, 2 min). TMT labeled peptides were re-dissolved in buffer A (300 μL, 95% H₂O, 5% CH₃CN, 0.1% FA) and loaded onto pre-equilibrated spin columns for high pH fractionation. The columns were spun down (2,000 g, 2 min) and the flow through was used to wash the original Eppendorf tube and passed through the spin column again (2,000 g, 2 min). The column was then washed with buffer A (300 μL, 2,000 g, 2 mM) and 10 mM aqueous NH₄HCO₃ containing 5% CH₃CN (300 μL, 2,000 g, 2 min), and the flow through was discarded. The peptides were eluted from the spin column into fresh Eppendorf tubes (2.0 mL) with a series of NH₄HCO₃/CH₃CN buffers (2000 g, 2 min).

### Whole Proteome TMT (TMT-exp) Sample Preparation

Freshly isolated T cells (1.6×10⁷ cells, 2×10⁶ cells/mL in RPMI media) were treated with compound or DMSO for 24 h, pelleted (600 g, 5 min), and washed with PBS (1×10 mL). The cells were then transferred to an Eppendorf tube in additional PBS (1 mL), pelleted (600 g, 5 mM), flash frozen, and kept at −80° C. until further analysis. On the first day of the whole proteome TMT protocol, the cells were thawed on ice and lysed in lysis buffer (150 μL, 1 tablet of Roche complete, mini, EDTA-free Protease Inhibitor Cocktail dissolved in 10 mL of PBS) using a probe sonicator (2×8 pulses). Protein concentration was adjusted to 2.0 mg/mL and the samples (100 μL) were transferred to new Eppendorf tubes (1.5 mL) containing urea (48 mg/tube, final urea concentration: 8 M). DTT (5 μL, 200 mM fresh stock in H₂O, final DTT concentration: 10 mM) was then added to the tubes and the samples were incubated at 65° C. for 15 min. Following this incubation, iodoacetamide (5 μL, 400 mM fresh stock in H₂O, final IA concentration: 20 mM) was added and the samples were incubated in the dark at 37° C. with shaking for 30 min. Ice-cold MeOH (600 μL), CHCl₃ (200 μL), and H₂O (500 μL) were then added, the mixture was vortexed and centrifuged (10,000 g, 10 min, 4° C.) to afford a protein disc at the interface of CHCl₃ and aqueous layers. The top layer was aspirated without perturbing the disk, additional MeOH (600 μL) was added and the proteins were pelleted (10,000 g, 10 min, 4° C.) and used in the next step or stored at −80° C. overnight.

### Whole Proteome TMT LysC and Trypsin Digestion

The resulting protein pellets were resuspended in EPPS buffer (160 μL, 200 mM, pH 8) using a probe sonicator (2×6 pulses). LysC solution (4 μL/sample, 20 μg in 40 μL of HPLC grade water) was added and the samples were incubated at 37° C. with shaking for 2 h. Trypsin (10 μL, 0.5 μg/μL in trypsin buffer) and CaCl₂ (1.8 μL, 100 mM in H₂O) were then added and the samples were incubated at 37° C. with shaking overnight.

### Whole Proteome TMT Labeling With TMT Tags

The sample became clear after the overnight digestion. At this point, peptide concentration was determined using microBCA assay (Thermo Scientific) according to manufacturer's instructions. For each sample, a volume corresponding to 25 μg of peptides was transferred to a new Eppendorf tube and the total volume was brought up to 35 μL with EPPS buffer (200 mM, pH 8). The samples were diluted with CH₃CN (9 μL) and incubated with the corresponding TMT tags (3 μL/sample, 20 μg/μL) at rt for 30 mM. The TMT tag treatment (3 μL/sample, 20 μg/μL, 30 min) was repeated, after which the tags were quenched by the addition of hydroxylamine (6 μL, 5% in H₂O). Following a 15 min incubation at rt, formic acid was added (2.5 μL, final FA concentration: 5%) and the samples were stored at −80° C. until further analysis.

### Whole Proteome TMT Ratio Check and High pH Fractionation

Small aliquots (2 μL) from each channel were combined in a separate Eppendorf tube and dried using SpeedVac vacuum concentrator. The residue was re-dissolved in Buffer A (20 μL) and desalted using C18 stage tips (made in-house using 200 μL pipette tips and C18 discs (3M Empore)). Briefly, the stage-tip was activated by passing MeOH (2×50 μL) through the stage tip and washed with Buffer B (2×50 μL, 5% H₂O, 95% CH₃CN, 0.1% FA), followed by Buffer A (2×50 μL, 5% CH₃CN/95% H₂O, 0.1% FA). The sample was then loaded and the stage-tip was washed with Buffer A. The sample was eluted into a new Eppendorf tube with Buffer B (2×50 μL) and dried using SpeedVac vacuum concentrator. The residue was re-dissolved in Buffer A (10 μL) and analyzed by mass-spectrometry using the following LC-MS gradient: 5% buffer B in buffer A from 0-15 min, 5-15% buffer B from 15-17.5 min, 15-35% buffer B from 17.5-92.5 min, 35-95% buffer B from 92.5-95 min, 95% buffer B from 95-105 min, 95-5% buffer B from 105-107 min, and 5% buffer B from 107-125 min (buffer A: 95% H₂O, 5% CH₃CN, 0.1% FA; buffer B: 5% H₂O, 95% CH₃CN, 0.1% FA) and standard MS3-based quantification described below. Ratios were determined from the average peak intensities corresponding to each channel For a ten-plex experiment, samples (20 μL/channel, final volumes adjusted based on the determined ratios) were combined in a new low binding Eppendorf tube (1.5 mL) and dried using SpeedVac. The residue was subjected to high pH fractionation as described above to yield 7 fractions which were re-suspended in buffer A (24 μL/sample) and analyzed by liquid chromatography tandem mass-spectrometry.

### TMT-ABPP and Whole Proteome TMT Liquid Chromatography-Mass-Spectrometry (LC-MS) Analysis

Samples were analyzed by liquid chromatography tandem mass-spectrometry using an Orbitrap Fusion mass spectrometer (Thermo Scientific) coupled to an UltiMate 3000 Series Rapid Separation LC system and autosampler (Thermo Scientific Dionex). The peptides were eluted onto a capillary column (75 μm inner diameter fused silica, packed with C18 (Waters, Acquity BEH C18, 1.7 μm, 25 cm) and separated at a flow rate of 0.25 μL/min using the following gradient: 5% buffer B in buffer A from 0-15 min, 5-35% buffer B from 15-155 min, 35-95% buffer B from 155-160 min, 95% buffer B from 160-169 min, 95-5% buffer B from 169-170 min, and 5% buffer B from 170-200 min (buffer A: 95% H₂O, 5% acetonitrile, 0.1% FA; buffer B: 5% H₂O, 95% CH₃CN, 0.1% FA). The voltage applied to the nano-LC electrospray ionization source was 1.9 kV. Data was acquired using an MS3-based TMT method. Briefly, the scan sequence began with an MS1 master scan (Orbitrap analysis, resolution 120,000, 400-1700 m/z, RF lens 60%, automatic gain control [AGC] target 2E5, maximum injection time 50 ms, centroid mode) with dynamic exclusion enabled (repeat count 1, duration 15s). The top ten precursors were then selected for MS2/MS3 analysis. MS2 analysis consisted of: quadrupole isolation (isolation window 0.7) of precursor ion followed by collision-induced dissociation (CID) in the ion trap (AGC 1.8E4, normalized collision energy 35%, maximum injection time 120 ms). Following the acquisition of each MS2 spectrum, synchronous precursor selection (SPS) enabled the selection of up to 10 MS2 fragment ions for MS3 analysis. MS3 precursors were fragmented by HCD and analyzed using the Orbitrap (collision energy 55%, AGC 1.5E5, maximum injection time 120 ms, resolution was 50,000). For MS3 analysis, we used charge state-dependent isolation windows. For charge state z=2, the MS isolation window was set at 1.2; for z=3-6, the MS isolation window was set at 0.7. The MS2 and MS3 files were extracted from the raw files using RAW Converter (version 1.1.0.22; available at hap://fields.scripps.edu/rawconv/), uploaded to Integrated Proteomics Pipeline (IP2), and searched using the ProLuCID algorithm (publicly available at http://fields.scripps.edu/downloads.php) using a reverse concatenated, non-redundant variant of the Human UniProt database (release-2012_11). Cysteine residues were searched with a static modification for carboxyamidomethylation (+57.02146) and up to one differential modification for the desthiobiotin (DTB) tag (+398.2529). N-terminus and lysine were also searched with a static modification corresponding to the TMT tag (+229.1629). Peptides were required to be at least 6 amino acids long, to have at least one tryptic terminus, and to contain the DTB modification. ProLuCID data was filtered through DTASelect (version 2.0) to achieve a peptide false-positive rate below 1%. The MS3-based peptide quantification was performed with reporter ion mass tolerance set to 20 ppm with Integrated Proteomics Pipeline (IP2).

### TMT-ABPP R Value Calculation for Broad Ligandability Data

At individual TMT experiment level, the following filters were applied to remove low-quality peptides: removal of non-unique peptides; removal of half-tryptic peptides; removal of peptides with more than one internal miscleaved sites; removal of peptides with low (<20,000) sum of reporter ion intensities for either expanded or activated control channels; removal of peptides with high variation between the replicate control channels (coefficient of variance >0.5), and peptides corresponding to the lower average reporter ion intensity control channels (activated vs expanded) if the difference in the average reporter ion intensity between expanded and activated control channels was more than two-fold. R-value (DMSO-treated vs. KB02/KB05-treated) for each peptide entry was calculated using the reporter ion intensities of DMSO and KB02/KB05 treated TMT channels for each treatment group with a maximum ratio cap of 20. Once the R values were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were used for further data processing. Peptides with high donor variation (R>5 for one donor, while R<2 for the other donor) were discarded (<1%), then the R values of replicate channels of the same condition were averaged to obtain the final reported data. A cysteine was required to be quantified in at least two TMT channels for each proteomic fraction to be reported.

### TMT-ABPP R Value Calculation for Elaborated Compounds Dataset

At individual TMT experiment level, the following filters were applied to remove low-quality peptides: removal of non-unique peptides; removal of half-tryptic peptides; removal of peptides with more than one internal miscleaved site; removal of peptides with low (<10,000) sum of reporter ion intensities for control channels, and peptides with high variation between the replicate control channels (coefficient of variance >0.5). R values (compound-treated vs. DMSO-treated) for each peptide entry were calculated using the reporter ion intensities of DMSO and compound treated TMT channels for each treatment group with a maximum ratio cap of 20. Once the R values for each peptide entry were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were used for further data processing. The R values of replicate channels of the same condition were averaged to obtain the final reported data with the requirement that all included peptides have been quantified in at least two individual experiments.

### Whole Proteome Protein Ratios Calculation for Elaborated Compounds Dataset

At individual TMT experiment level, the following filters were applied to remove low-quality peptides: removal of non-unique peptides; removal of half-tryptic peptides; removal of peptides with more than one internal miscleaved site; removal of cysteine-containing peptides; removal of peptides with low (<10,000) sum of reporter ion intensities for control channels, and peptides with high variation between the replicate control channels (coefficient of variance >0.5). R values (compound-treated vs. DMSO-treated) were calculated using the reporter ion intensities of compound and DMSO treated TMT channels for each treatment group. Then the ratios of all peptides of a protein were averaged to be reported as the final protein ratio. Proteins were required to have at least two unique quantified peptides in each experiment and were quantified in at least two independent experiments.

### Whole Proteome Protein Ratios Calculation for State-Dependent Dataset

The MS3-based peptide quantification was performed with reporter ion mass tolerance set to 20 ppm with Integrated Proteomics Pipeline (IP2). At individual TMT experiment level, the following filters were applied to remove low-quality peptides: removal of non-unique peptides; removal of half-tryptic peptides; removal of peptides with more than one internal miscleaved sites; removal of peptides with low (<10,000) sum of reporter ion intensities (5 channels/donor), and peptides with high variation between either of the replicate channels for expanded or activated T cells (coefficient of variance >0.5). R values (activated vs. expanded) for each peptide entry were calculated using the average reporter ion intensities of activated and expanded TMT channels. Then the ratios of all quantified peptides for a protein were averaged to obtain the final protein ratio. Proteins were required to have at least two unique quantified peptides in each experiment.

### TMT-ABPP R Value Calculation for Cysteine State-Dependent Reactivity Dataset

At individual TMT experiment level, the following filters were applied to remove low-quality peptides: removal of non-unique peptides; removal of half-tryptic peptides; removal of peptides with more than one internal miscleaved site; removal of peptides with low (<10,000) sum of reporter ion intensities in both expanded or activated channels; removal of peptides with high variation (coefficient of variance >0.5) between the replicate expanded or activated channels if their sum of reporter ion intensities is greater than 5,000. R values (activated vs. expanded) for each peptide were calculated using the average reporter ion intensities of activated and expanded TMT channels. Once the R values were calculated, two types of grouping were performed to aggregate peptide quantification data: 1) overlapping peptides with the same modified cysteine (e.g., different charge states, high pH fractionation fractions, or tryptic termini) were grouped together, then their R values were averaged, and the shortest unique tryptic peptide was reported; 2) multiple modified cysteines on a tryptic peptide were grouped together, then the averaged R values were reported for further data processing. The median value derived from at least two biological replicates was reported as the final R value for each peptide with a maximum ratio cap of 20.

### Data Processing and Analysis for IA-DTB Reactivity Dataset

Proteins must have at least three unique quantified peptides in either particulate or soluble fraction in the TMT-ABPP experiments within the state-dependent dataset to be analyzed. The fraction with the most quantified unique peptides was selected for analysis for each protein. If a protein had an equal number of unique quantified peptides in both fractions, the peptide R ratios (activated vs. expanded) from both fractions were averaged. To account for potential donor variations in protein expression level, proteins were required to have at least one peptide R ratio within 1.5-fold of the protein expression level measured in TMT-exp experiments (if available) and were excluded from the analysis if all peptide R ratios were greater than 2.0 or less than 0.5. For proteins with 5 or more quantified peptides, a cysteine was considered for potential change in reactivity if its peptide R value differed more than two-fold from both the median R value of all quantified cysteines on the same protein and from the protein expression level measured in TMT-exp experiments (if available). For proteins with three or four quantified peptides, a cysteine was considered for potential change in reactivity if its peptide R value differed more than two-fold from the protein expression level measured by TMT-exp data, with an additional requirement that the maximum peptide R ratio differed more than 2-fold from the minimum peptide R ratio. All the cysteines that passed the initial filters described above were manually curated to remove low quality profiles.

### General Methods

The crystal structures of the proteins were retrieved from the Protein Data Bank: TIR domain of MYD88 (PDB 4DOM), ERCC3 (PDB 5OF4), TMEM173 (PDB 6NT5). Hydrogens were added with Reduce, then were prepared using AutoDockTools following the standard AutoDock protocol. Reactive docking was performed following the protocol reported previously (Backus et al., 2016, Nature 534, 570-574). A grid box was defined for each cysteine: C168, C192, C216, C203, C247 and C280 (size x: 60, y: 60, z: 60 points).

For the flexible side chain covalent method, ligands were modelled attached to the alkylated residue via covalent bond, then processed following the covalent docking protocol (available online at http://autodock.scripps.edu/resources/covalentdocking) to be modeled as flexible during the docking. All dockings were performed using AutoDock 4.2, generating 100 poses using the default LGA parameters. Poses with the best energy score were selected and analyzed. Figures were generated using Pymol.(The PyMOL Molecular Graphics System, Version 2.0 Schrodinger, LLC.)

### Example 7

### Chemical Proteomic Map of Cysteine Reactivity in Activated T Cells

Some of the discovered changes in activated T cells occur in general biochemical pathways associated with cell proliferation, while others reflect immune-restricted processes. The extent to which these types of activation state-dependent changes in the biochemistry of T cells might also create a landscape of new targets for chemical probes that regulate T cell function remains largely unexplored. The global scale profiling was addressed by using activity-based protein profiling (ABPP) methods to quantify cysteine reactivity and electrophilic small-molecule interactions in primary human T cells activated by T cell receptor (TCR) stimulation.

Primary human T cells are isolated from human blood, activated by exposure to anti-CD3 and anti-CD28 antibodies for three days, and then expanded in culture in the presence of IL2. Cysteine reactivity changes are measured by treating proteomic lysates from activated or expanded control T cells with a broad-spectrum, cysteine-directed iodoacetamide-desthiobiotin probe (1A-DTB), protease digestion of the IA-DTB-treated proteomes, streptavidin enrichment of IA-DTB-labeled cysteine-containing peptides, and quantitative, multiplexed LC-MS-based proteomic analysis using tandem mass tags (TMT, 10-plex experiments). These cysteine reactivity profiles are then integrated with complementary proteomic experiments measuring protein expression changes in control versus activated T cells. In total, more than 4800 proteins were quantified in expression-based (TMT-exp) proteomic experiments, the vast majority of which (˜80%) were also quantified by cysteine reactivity profiling (TMT-ABPP), and nearly a quarter of these proteins qualified as “immune-relevant”, based on immune cell-enriched expression profiles derived from public databases and/or human genetic (OMIM) evidence of contributing to immune-related disorders. A protein is considered to show altered expression if its abundance was elevated or reduced by >two-fold in activated T cells, and ˜1100 proteins satisfied this requirement.

A distinct set of proteins harbored cysteine reactivity changes that differed substantially from the corresponding expression profiles for these proteins in activated T cells. These cysteine reactivity changes were found in immune-relevant proteins and featured functional sub-groups that may reflect the diverse modulation of cellular biochemistry in activated T cells. For example, a number of catalytic and active-site cysteines in proteins involved in redox regulation showed much greater reactivity in activated T cells, possibly reflecting the higher intracellular reducing potential of these cells furnished, at least in part, by increases in glutathione production. Reactivity changes are also found for cysteines in the metal-binding domains of proteins, with one prominent example being the immune-relevant protein L-plastin (LCP1), which is a calcium-regulated actin-binding protein that participates in remodeling of the actin cytoskeleton during T cell activation. Calcium binding decreases the ability of LCP-1 to bind actin by inducing structural changes to the EF-hand motif of LCP-1, in particular, in α-helices 2 and 3 surrounding C42, which showed increased reactivity in activated T cells. Additional reactivity changes are observed for cysteines in DNA or RNA-binding domains and at the sites of protein-protein interactions (e.g., EZH2, NEDD9, TNFAIP3), as well as cysteines proximal to cofactor- and metabolite-binding sites. These cysteine reactivity changes may reflect a landscape of dynamic intermolecular interactions occurring in activated T cells that, in turn, impinge upon the reactivity of cysteines. As one example, C269 in isocitrate dehydrogenase 1 (IDH1) undergoes a dramatic increase in reactivity in activated T cells, which could reflect changes in cofactor (NADP) and/or substrate (isocitrate) binding that promote a structural rearrangement in residues 271-277 (Xu et al., 2004), which may, in turn, alter the reactivity of C269.

### Chemical Proteomic Map of Cysteine Ligandability in Human T Cells

An efficient strategy to globally assess the ligandability of cysteines in native biological systems that leverages broad-reactivity was recently described. Electrophilic small-molecule fragments are referred to as “scouts”. Two scout fragments bearing a chloroacetamide (KB02) or acrylamide (KB05)—reactive groups frequently found in covalent chemical probes and drugs—were used to construct in-depth cysteine ligandability maps across primary human T cells in both resting and activated states. Scout fragment-cysteine interactions were analyzed using two complementary chemical proteomic methods that provided a balance of confidence in quantitative accuracy (isoTOP-ABPP) with greater multiplexing capacity (TMT-ABPP). Both proteomic methods provided similar ratio (R) values (DMSO/scout fragment (500 μM, 1 h)) for cysteines in T cell proteomes, with the MS3-based quantification used in TMT-ABPP resulting in mild ratio compression , which was countered by a substantial increase in proteomic coverage compared to isoTOP-ABPP. Designated cysteines were ligandable if they showed an R value of ≥5 as measured by either isoTOP-ABPP or TMT-ABPP.

The cysteine reactivity proteomic data from combined isoTOP-ABPP and TMT-ABPP experiments with scout fragments and active compounds as well as isoTOP-ABPP hyperreactivity experiments are shown in Tables 1 and 2 below.

Among the liganded cysteines were several targeted by existing covalent probes and drug candidates, including those being pursued for immunological disorders (e.g., C909 in JAK3, C528 in XPO1), underscoring the potential for ABPP to “rediscover” established druggable sites on immune-relevant proteins. Ligandable cysteines were also well-represented within the subset of proteins showing expression and/or cysteine reactivity changes in activated T cells, where cysteines with altered reactivity showed a greater propensity for liganding by scout fragments. As one example, the discovery of a ligandable cysteine (C93) in programmed cell death protein 1 (PDCD1 or PD-1) was noted, which was only observed in activated T cells, and likely reflects the induced expression of this key immune checkpoint protein following T cell stimulation. Ligandable cysteines showing reactivity-based changes included the catalytic cysteine in the deubiqutinase USP16 (C205), which has been shown to regulate hematopoietic stem cell differentiation.

Fragment-based screening, whether performed on a single protein of interest or more broadly across the proteome as described herein, offers advantages for discovering hit compounds for challenging protein classes. Nonetheless, progressing fragments to more advanced chemical probes and ultimately drugs can be confounded by a variety of factors, including the low-affinity and promiscuity of initial hits and the tractability of fragment-binding sites on proteins.

A multidimensional screen can be preformed of a focused library of structurally elaborated electrophilic small molecules to identify compounds that suppress T cell activation at low-μM concentrations without causing cytotoxicity. Primary human T cells isolated from the blood of healthy donors can be activated with anti-human antibodies against CD3 and CD28 antigens in the presence of an in-house collection of electrophilic compounds, where suppression of T cell activation was measured 24 h later by reductions in secreted IL-2 and IFN-γ and in the expression of the cell surface markers CD69 and CD25. The viability of T cells can be monitored by flow cytometry using near-IR live-dead stain. 17 compounds, along with the immunosuppressive drug DMF, which was used as a positive control, were found to substantially suppress T cell activation (>65%) without causing cytotoxicity (viability >85%).

### Chemical Proteomic Analysis of Immunosuppressive Electrophilic Compounds

The targets of active compounds were mapped in primary human T cells by ABPP. Cysteine reactivity profiles were acquired for T cells treated with active compounds (10-20 μM for elaborated compounds BKP-21/25 and EV-3/93; 50 μM for DMF) for 3 h. On average, 10,000-12,000 cysteines were quantified for each compound (aggregated across at least 6 biological replicates, where it was required that a cysteine was quantified in at least two replicates for interpretation), of which only a modest fraction (0.2-1.0%) was substantially altered in reactivity by compound treatment (RDMSO/compound >4). There is a distinct cysteine reactivity profiles of each hit compound, which engaged largely non-overlapping sets of targets that originated from diverse structural and functional classes and included several immune-relevant proteins.

The vast majority of cysteines liganded by described compounds can also be engaged by scout fragments, underscoring the potential for fragment profiling to discover tractable sites of ligandability across the human proteome that can also be targeted by more elaborated electrophiles with improved potency (low-μM) and credible SARs. Molecular modeling can be performed on the structures of proteins containing cysteines targeted by hit compounds, to predict binding pockets within 5 Å of the liganded cysteines.

### Electrophilic Compound-Dependent Degradation of Proteins: The Acrylamide and Chloroacetamide can Promotes Degradation of the Protein

As noted previously, the structurally related acrylamides can be used to suppress T cell activation, but by apparently different mechanisms. Acrylamide and chloroacetamide compounds impaired T cell activity by inhibiting proteins or inhibit cell signaling pathway. A survey of the cysteines preferentially engaged by acrylamide and chloroacetamide did not reveal obvious candidate proteins within the NF-κB pathway as potential targets of relevance for the former compound.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods within the scope of these claims and their equivalents be covered thereby.

Lengthy table referenced here
US20240123078A1-20240418-T00001
Please refer to the end of the specification for access instructions.

TABLE 2
Cysteine reactivity proteomic data from combined isoTOP-ABPP and TMT-ABPP experiments with
scout fragments and active compounds as well as isoTOP-ABPP hyperreactivity experiments.

| K4_500 | L4_500 | L4_100 | B9_500 | | | | | |
|---|---|---|---|---|---|---|---|---|
| μM | μM | μM | μM | B9_100 | Identifier | Uniprot | Description | Residues |
| | | | | 100 | Q86TI2_CPESGEHY | Q86TI2 | DPP9 | C844 |
| | | | | | EVTLLHFLQEYL | | Dipeptidyl peptidase | |
| | | | | | | | 9 | |
| | | | | 100 | Q9Y2S7_DCPHISQR | Q9Y2S7 | POLDIP2 | C143 |
| | | | | | | | Polymerase delta- | |
| | | | | | | | interacting protein 2 | |
| | | | | 99.9317 | P42680 GCLLNFLR | P42680 | TEC Tyrosine- | C449 |
| | | | | | | | protein kinase Tec | |
| 3.32 | −21.9512 | 12.2537 | 99.8498 | 99.8839 | P16455_GNPVPILIP | P16455 | MGMT Methylated- | C145 |
| | | | | | CHR | | DNA--protein- | |
| | | | | | | | cysteine | |
| | | | | | | | methyltransferase | |
| 29.0709 | 88.9926 | 88.0107 | 98.293 | 96.565 | P21580_TCQFCPQFR | P21580 | TNFAIP3 Tumor | C54, C57 |
| | | | | | | | necrosis factor alpha- | |
| | | | | | | | induced protein 3 | |
| 16.17 | | | | 95.36 | Q9NQR4_VGLGICY | Q9NQR | NIT2 Omega- | C153 |
| | | | | | DMR | 4 | amidase NIT2 | |
| | | | | 94.3042 | Q96RU2_CEVWGEN | Q96RU2 | USP28 Ubiquitin | C142 |
| | | | | | PNPNDWR | | carboxyl-terminal | |
| | | | | | | | hydrolase 28 | |
| 14.98 | | | | 94.22 | Q8NEC7_ACAEVSQ | Q8NEC7 | GSTCD Glutathione | C140 |
| | | | | | WTR | | S-transferase C- | |
| | | | | | | | terminal domain- | |
| | | | | | | | containing protein | |
| 10.19 | | | | 92.6 | Q13613_DVMYICPF | Q13613 | MTMR1 | C117 |
| | | | | | MGAVSGTLTVTDF | | Myotubularin-related | |
| | | | | | K | | protein 1 | |
| | | | | 91.08 | Q96AB3_SVLLCGIE | Q96AB3 | ISOC2 | C114 |
| | | | | | AQACILNTTLDLLD | | Isochorismatase | |
| | | | | | R | | domain-containing | |
| | | | | | | | protein 2, | |
| | | | | | | | mitochondrial | |
| 6.81 | | | | 90.46 | O95881_SWCGACK | O95881 | TXNDC12 | C66 |
| | | | | | | | Thioredoxin domain- | |
| | | | | | | | containing protein 12 | |
| 6.57 | | | | 89.91 | Q5TFE4_HFLSDTG | Q5TFE4 | NT5DC1 5′- | C119 |
| | | | | | MACR | | nucleotidase domain- | |
| | | | | | | | containing protein 1 | |
| 11.64 | | | | 89.65 | Q9UL40_NQCLFTNT | Q9UL40 | ZNF346 Zinc finger | C68 |
| | | | | | QCK | | protein 346 | |
| | | | | 89.23 | P52333_LVMEYLPS | P52333 | JAK3 Tyrosine- | C909 |
| | | | | | GCLR | | protein kinase JAK3 | |
| | | | | 89.04 | Q96ME1_VGVQSCP | Q96ME1 | FBXL18 F-box/LRR- | C459, |
| | | | | | SPFSGQACPQPSSVF | | repeat protein 18 | C468 |
| | | | | | WSLLK | | | |
| | | | | 88.96 | Q13315_ATFAYISN | Q13315 | ATM Serine-protein | C1396 |
| | | | | | CHK | | kinase ATM | |
| 5.68487 | 97.5383 | 90.2621 | 99.4535 | 88.2428 | P16455_VVCSSGAV | P16455 | MGMT Methylated- | C150 |
| | | | | | GNYSGGLAVK | | DNA--protein- | |
| | | | | | | | cysteine | |
| | | | | | | | methyltransferase | |
| 6.75 | | | | 88.13 | Q13490_YDFSCELY | Q13490 | BIRC2 Baculoviral | C45 |
| | | | | | R | | IAP repeat- | |
| | | | | | | | containing protein 2 | |
| 46.06 | | | | 86.25 | P30519_CPFYAAEQ | P30519 | HMOX2 Heme | C265, |
| | | | | | DKGALEGSSCPFR | | oxygenase 2 | C282 |
| | | | | 86.25 | P30519_GALEGSSCP | P30519 | HMOX2 Heme | C282 |
| | | | | | FR | | oxygenase 2 | |
| 10.99 | | | | 85.67 | P07237_NVFVEFYA | P07237 | P4HB Protein | C397, |
| | | | | | PWCGHCK | | disulfide-isomerase | C400 |
| | | | | 85.58 | P07858_DQGSCGSC | P07858 | CTSB Cathepsin B | C105, |
| | | | | | WAFGAVEAISDR | | | C108 |
| 18.2 | | | | 83.03 | P07237_YLLVEFYA | P07237 | P4HB Protein | C53, C56 |
| | | | | | PWCGHCK | | disulfide-isomerase | |
| 12.15 | | | | 82.95 | P78417_FCPFAER | P78417 | GSTO1 Glutathione | C32 |
| | | | | | | | S-transferase omega- | |
| | | | | | | | 1 | |
| 6.5 | | | | 82.88 | O15050_GSFEALSCP | O15050 | TRANK1 TPR and | C1319 |
| | | | | | HGR | | ankyrin repeat- | |
| | | | | | | | containing protein 1 | |
| | | | | 82.42 | Q9Y4A5_IYPYLVM | Q9Y4A5 | TRRAP | C3535 |
| | | | | | NDACLTESR | | Transformation/ | |
| | | | | | | | transcription domain- | |
| | | | | | | | associated protein | |
| 15.67 | | | | 81.35 | P04818_DLPLMALP | P04818 | TYMS Thymidylate | C195, |
| | | | | | PCHALCQFYVVNSE | | synthase | C199 |
| | | | | | LSCQLYQR | | | |
| | | | | 81.29 | Q86X76_THLCDVEI | Q86X76 | NIT1 Nitrilase | C165, |
| | | | | | PGQGPMCESNSTM | | homolog 1 | C176 |
| | | | | | PGPSLESPVSTPAG | | | |
| | | | | | K | | | |
| 15.43 | | | | 80.68 | Q02252_CMALSTAV | Q02252 | ALDH6A1 | C317 |
| | | | | | LVGEAK | | Methylmalonate- | |
| | | | | | | | semialdehyde | |
| | | | | | | | dehydrogenase | |
| 8.79 | | | | 79.96 | Q9Y676_LLEQFVCA | Q9Y676 | MRPS18B 28S | C128 |
| | | | | | HTGIIFYAPYTGVC | | ribosomal protein | |
| | | | | | VK | | S18b, mitochondrial | |
| 71.61 | | | | 79.82 | Q9H7Z7_TCPFCSK | Q9H7Z7 | PTGES2 | C110 |
| | | | | | | | Prostaglandin E | |
| | | | | | | | synthase 2 | |
| | | | | 79.73 | P15121_VCALLSCTS | P15121 | AKR1B1 Aldose | C299, |
| | | | | | HK | | reductase | C304 |
| 0.58 | | | | 79.5 | O95456_NMQITILTC | O95456 | PSMG1 Proteasome | C169 |
| | | | | | R | | assembly chaperone | |
| | | | | | | | 1 | |
| | | | | 79.4 | Q99757_VVNSETPV | Q99757 | TXN2 Thioredoxin, | C90, C93 |
| | | | | | VVDFHAQWCGPCK | | mitochondrial | |
| 41.52 | | | | 79.2 | Q6NUM9_LHPCVM | Q6NUM | RETSAT All-trans- | C547 |
| | | | | | ASLR | 9 | retinol 13,14- | |
| | | | | | | | reductase | |
| 22.4158 | 98.2637 | 96.4386 | 98.8468 | 78.9759 | P29597_LPEPSCPQL | P29597 | TYK2 Non-receptor | C838 |
| | | | | | ATLTSQCLTYEPTQ | | tyrosine-protein | |
| | | | | | RPSFR | | kinase TYK2 | |
| | | | | 78.65 | P57075 YYRPDLNF | P57075 | UBASH3A | C435 |
| | | | | | PCSLPR | | Ubiquitin-associated | |
| | | | | | | | and SH3 domain- | |
| | | | | | | | containing protein A | |
| 16.71 | | | | 78.37 | Q9NRW3_LYYFQYP | Q9NRW | APOBEC3C | C130 |
| | | | | | CYQEGLR | 3 | Probable DNA dC- | |
| | | | | | | | dU-editing enzyme | |
| | | | | | | | APOBEC-3C | |
| | | | | 78.26 | Q07960_IIVFSACR | Q07960 | ARHGAP1 Rho | C91 |
| | | | | | | | GTPase-activating | |
| | | | | | | | protein 1 | |
| | | | | 77.67 | Q9Y485 SIMMYAC | Q9Y485 | DMXL1 DmX-like | C551 |
| | | | | | TK | | protein 1 | |
| | | | | 77.6337 | Q9NZJ0_GHQCSLK | Q9NZJ0 | DTL Denticleless | C141 |
| | | | | | | | protein homolog | |
| 11.34 | | | | 77.13 | Q9HBL8_LPCYFENL | Q9HBL8 | NMRAL1 NmrA-like | C154 |
| | | | | | LSHFLPQK | | family domain- | |
| | | | | | | | containing protein 1 | |
| | | | | 77.12 | Q9BZH6_TVRPFSM | Q9BZH6 | WDR11 WD repeat- | C364 |
| | | | | | VCCPVNENAAALV | | containing protein 11 | |
| | | | | | VSDGR | | | |
| 29.03 | | | | 76.34 | Q8IZ69_VIGVELCPE | Q8IZ69 | TRMT2A tRNA | C463 |
| | | | | | AVEDAR | | (uracil-5-)- | |
| | | | | | | | methyltransferase | |
| | | | | | | | homolog A | |
| | | | | 76.2371 | P42858_QYTTTTCV | P42858 | HTT Huntingtin | C1441 |
| | | | | | QLQK | | | |
| | | | | 76.0802 | Q14145_CVLHVMN | Q14145 | KEAP1 Kelch-like | C151 |
| | | | | | GAVMYQIDSVVR | | ECH-associated | |
| | | | | | | | protein 1 | |
| | | | | 75.52 | Q9BYB4_LWQADCS | Q9BYB4 | GNB1L Guanine | C175 |
| | | | | | SR | | nucleotide-binding | |
| | | | | | | | protein subunit beta- | |
| | | | | | | | like protein 1 | |
| 12.94 | | | | 75.1 | P49189_GALMANFL | P49189 | ALDH9A1 4- | C288, |
| | | | | | TQGQVCCNGTR | | trimethylaminobutyraldehyde | C289 |
| | | | | | | | dehydrogenase | |
| −1.89 | | | | 75.02 | Q86X76_IGLAVCYD | Q86X76 | NIT1 Nitrilase | C203 |
| | | | | | MR | | homolog 1 | |
| 5.72 | | | | 74.73 | Q9HA64_ATGHSGG | Q9HA64 | FN3KRP | C24 |
| | | | | | GCISQGR | | Ketosamine-3-kinase | |
| | | | | 73.96 | Q96GX2_LPLCSLPG | Q96GX2 | ATXN7L3B Putative | C75 |
| | | | | | EPGNGPDQQLQR | | ataxin-7-like protein | |
| | | | | | | | 3B | |
| | | | | 73.9 | O75691_LPPQSCLLL | O75691 | UTP20 Small subunit | C2058 |
| | | | | | PPTPVR | | processome | |
| | | | | | | | component 20 | |
| | | | | | | | homolog | |
| | | | | 73.81 | Q9BY49_SLALEWA | Q9BY49 | PECR Peroxisomal | C191 |
| | | | | | CSGIR | | trans-2-enoyl-CoA | |
| | | | | | | | reductase | |
| | | | | 73.7291 | Q9NZJ0_ICTYFHR | Q9NZJ0 | DTL Denticleless | C710 |
| | | | | | | | protein homolog | |
| 0.15 | | | | 73.25 | Q96MX3_FVLSCSLL | Q96MX | ZNF48 Zinc finger | C288 |
| | | | | | SHQR | 3 | protein 48 | |
| 14.53 | | | | 73.13 | Q6YN16_QHCAYTI | Q6YN16 | HSDL2 | C166 |
| | | | | | AK | | Hydroxysteroid | |
| | | | | | | | dehydrogenase-like | |
| | | | | | | | protein 2 | |
| 12.21 | | | | 72.89 | Q8WV74_LAGLTCS | Q8WV7 | NUDT8 Nucleoside | C207 |
| | | | | | GAEGLAR | 4 | diphosphate-linked | |
| | | | | | | | moiety X motif 8, | |
| | | | | | | | mitochondrial | |
| 45.88 | | | | 72.8 | O96017_TLGSGACG | O96017 | CHEK2 | C231 |
| | | | | | EVK | | Serine/threonine- | |
| | | | | | | | protein kinase Chk2 | |
| | | | | 72.68 | Q2VPK5_TPPGPCCS | Q2VPK5 | CTU2 Cytoplasmic | C433 |
| | | | | | PGVGWAQR | | tRNA 2-thiolation | C434 |
| | | | | | | | protein 2 | |
| | | | | 72.64 | P20073_VLIEILCTR | P20073 | ANXA7 Annexin A7 | C285 |
| −1.62 | | | | 72.34 | O43776_LMTDTINE | O43776 | NARS Asparagine-- | C438 |
| | | | | | PILLCR | | tRNA ligase, | |
| | | | | | | | cytoplasmic | |
| | | | | 72.19 | P45984_TACTNFMM | P45984 | MAPK9 Mitogen- | C177 |
| | | | | | TPYVVTR | | activated protein | |
| | | | | | | | kinase 9 | |
| | | | | 71.98 | Q9BRR9_SPPPGPAC | Q9BRR9 | ARHGAP9 Rho | C210 |
| | | | | | PLLQR | | GTPase-activating | |
| | | | | | | | protein 9 | |
| 3.69 | | | | 71.94 | O00541_SLCIGATY | O00541 | PES1 Pescadillo | C361 |
| | | | | | DVTDSR | | homolog | |
| | | | | 71.64 | Q96Q05_LDFSCQEN | Q96Q05 | TRAPPC9 | C800 |
| | | | | | LLQDLSDDGISVSG | | Trafficking protein | |
| | | | | | FPLSSPFR | | particle complex | |
| | | | | | | | subunit 9 | |
| 1.71 | | | | 71.33 | Q8IZL8_SAPHLPGL | Q8IZL8 | PELP1 Proline-, | C71 |
| | | | | | MCLLR | | glutamic acid- and | |
| | | | | | | | leucine-rich protein 1 | |
| 19.52 | | | | 70.66 | Q6P087_ESSGLVLL | Q6P087 | RPUSD3 RNA | C147 |
| | | | | | SSCPQTASR | | pseudouridylate | |
| | | | | | | | synthase domain- | |
| | | | | | | | containing protein 3 | |
| | | | | 70.44 | Q9Y4K1_ISSVQPICL | Q9Y4K1 | AIM1 Absent in | C1404 |
| | | | | | DSFTGPR | | melanoma 1 protein | |
| 21.91 | | | | 69.87 | Q5JPI3_CPFVENTW | Q5JPI3 | C3orf38 | C259 |
| | | | | | K | | Uncharacterized | |
| | | | | | | | protein C3orf38 | |
| −0.63 | | | | 69.8 | Q5T4S7_DVPVEALT | Q5T4S7 | UBR4 E3 ubiquitin- | C4049 |
| | | | | | TVKPYCNEIHAQAQ | | protein ligase UBR4 | |
| | | | | | LWLK | | | |
| 8.32 | | | | 69.31 | P25786_PYGVGLLIA | P25786 | PSMA1 Proteasome | C148, |
| | | | | | GYDDMGPHIFQTCP | | subunit alpha type-1 | C156 |
| | | | | | SANYFDCR | | | |
| 30.48 | | | | 69.23 | Q8TBP6_IAPACAIM | Q8TBP6 | SLC25A40 Solute | C314 |
| | | | | | ISTYEFGK | | carrier family 25 | |
| | | | | | | | member 40 | |
| −0.58 | | | | 68.58 | P62888_VCTLAIIDP | P62888 | RPL30 60S | C92 |
| | | | | | GDSDIIR | | ribosomal protein | |
| | | | | | | | L30 | |
| 2.38 | | | | 68.2 | O95644_GLGACTLL | O95644 | NFATC1 Nuclear | C228 |
| | | | | | GSPR | | factor of activated T- | |
| | | | | | | | cells, cytoplasmic 1 | |
| | | | | 67.98 | Q9H7Z6_GQHVICVT | Q9H7Z6 | KAT8 Histone | C416 |
| | | | | | PK | | acetyltransferase | |
| | | | | | | | KAT8 | |
| 45.45 | | | | 67.62 | Q6PJG6_THCPYAV | Q6PJG6 | BRAT1 BRCA1- | C673 |
| | | | | | ALPEVAPAQPLTEA | | associated ATM | |
| | | | | | LR | | activator 1 | |
| | | | | 67.57 | O76075_VLGSMCQR | O76075 | DFFB DNA | C194 |
| | | | | | | | fragmentation factor | |
| | | | | | | | subunit beta | |
| | | | | 67.16 | Q9NZ32_IPDWCSLN | Q9NZ32 | ACTR10 Actin- | C388 |
| | | | | | NPPLEMMFDVGK | | related protein 10 | |
| 4.76 | | | | 66.82 | Q12769_ALECFCQA | Q12769 | NUP160 Nuclear | C929 |
| | | | | | ASEVGKEEFLDR | | pore complex protein | C931 |
| | | | | | | | Nup160 | |
| 31.15 | | | | 66.79 | Q86UL3_SKLPILIFP | Q86UL3 | AGPAT6 Glycerol-3- | C325 |
| | | | | | EGTCINNTSVMMF | | phosphate | |
| | | | | | K | | acyltransferase 4 | |
| 20.44 | | | | 66.06 | P78527_QCLPSLDLS | P78527 | PRKDC DNA- | C1499, |
| | | | | | CK | | dependent protein | C1507 |
| | | | | | | | kinase catalytic | |
| | | | | | | | subunit | |
| | | | | 65.9 | Q92922_NLTGDVCA | Q92922 | SMARCC1 | C520 |
| | | | | | VMR | | SWI/SNF complex | |
| | | | | | | | subunit SMARCC1 | |
| | | | | 65.73 | Q15126_GWVFTPGV | Q15126 | PMVK | C156 |
| | | | | | DDAESECGLDNFG | | Phosphomevalonate | |
| | | | | | DFDWVIENHGVEQ | | kinase | |
| | | | | | R | | | |
| 12.6621 | 80.6157 | 42.1791 | 69.5373 | 64.9054 | P40763_FICVTPTTC | P40763 | STAT3 Signal | C712, |
| | | | | | SNTIDLPMSPR | | transducer and | C718 |
| | | | | | | | activator of | |
| | | | | | | | transcription 3 | |
| 9.88065 | | | | 64.8685 | Q86UT6_LNQPYCG | Q86UT6 | NLRX1 NLR family | C331 |
| | | | | | YAVGGSGVSATPA | | member X1 | |
| | | | | | QR | | | |
| 27.62 | | | | 64.76 | Q6NXE6_AFLDNPGI | Q6NXE6 | ARMC6 Armadillo | C297 |
| | | | | | LSELCGTLSR | | repeat-containing | |
| | | | | | | | protein 6 | |
| 22.69 | | | | 64.58 | Q15796_CVTIPSTCS | Q15796 | SMAD2 Mothers | C74, C81 |
| | | | | | EIWGLSTPNTIDQW | | against | |
| | | | | | DTTGLYSFSEQTR | | decapentaplegic | |
| | | | | | | | homolog 2 | |
| 3.18 | | | | 64.57 | Q9Y679_TGCVDLTI | Q9Y679 | AUP1 Ancient | C391 |
| | | | | | TNLLEGAVAFMPE | | ubiquitous protein 1 | |
| | | | | | DITK | | | |
| | | | | 64.56 | Q8IY21_CQFDEK | Q8IY21 | DDX60 Probable | C517 |
| | | | | | | | ATP-dependent RNA | |
| | | | | | | | helicase DDX60 | |
| | | | | 64.48 | Q13464_SEVNSDCL | Q13464 | ROCK1 Rho- | C29 |
| | | | | | LDGLDALVYDLDF | | associated protein | |
| | | | | | PALR | | kinase 1 | |
| | | | | 64.44 | P61970_NINDAWVC | P61970 | NUTF2 Nuclear | C114 |
| | | | | | TNDMFR | | transport factor 2 | |
| | | | | 64.39 | Q14289_VSEGMALQ | Q14289 | PTK2B Protein- | C180 |
| | | | | | LGCLELR | | tyrosine kinase 2-beta | |
| | | | | 63.68 | P32929_FLQNSLGA | P32929 | CTH Cystathionine | C252 |
| | | | | | VPSPIDCYLCNR | | gamma-lyase | |
| | | | | 63.5 | Q14586_SSCLTQHR | Q14586 | ZNF267 Zinc finger | C506 |
| | | | | | | | protein 267 | |
| | | | | 63.33 | P17480_WSQEDMLT | P17480 | UBTF Nucleolar | C33 |
| | | | | | LLECMK | | transcription factor 1 | |
| 7.77 | | | | 63.16 | O00267_SFAFLHCK | O00267 | SUPT5H | C626 |
| | | | | | | | Transcription | |
| | | | | | | | elongation factor | |
| | | | | | | | SPT5 | |
| −0.33 | | | | 62.52 | O75131_EALAQCVL | O75131 | CPNE3 Copine-3 | C506 |
| | | | | | AEIPQQVVGYFNTY | | | |
| | | | | | K | | | |
| −11.87 | | | | 62.44 | Q9BUP3_YSVFRPG | Q9BUP3 | HTATIP2 | C172 |
| | | | | | VLLCDR | | Oxidoreductase | |
| | | | | | | | HTATIP2 | |
| 34.2758 | | 13.8951 | 94.9822 | 62.3249 | Q15084_NVLDSEDV | Q15084 | PDIA6 Protein | C190, |
| | | | | | WMVEFYAPWCGH | | disulfide-isomerase | C193 |
| | | | | | CK | | A6 | |
| 8.05 | | | | 62.28 | P24666_SVLFVCLG | P24666 | ACP1 Low molecular | C13, C18 |
| | | | | | NICR | | weight | |
| | | | | | | | phosphotyrosine | |
| | | | | | | | protein phosphatase | |
| 53.39 | | | | 62.2 | Q3SXM5_CSWLVPS | Q3SXM | HSDL1 Inactive | C265 |
| | | | | | PK | 5 | hydroxysteroid | |
| | | | | | | | dehydrogenase-like | |
| | | | | | | | protein 1 | |
| | | | | 62.16 | Q8IU81_EQLLALSA | Q8IU81 | IRF2BP1 Interferon | C239 |
| | | | | | CAPFNVR | | regulatory factor 2- | |
| | | | | | | | binding protein 1 | |
| | | | | 62.11 | Q16875_VESIYLNV | Q16875 | PFKFB3 6- | C440 |
| | | | | | ESVCTHR | | phosphofructo-2- | |
| | | | | | | | kinase/fructose-2,6- | |
| | | | | | | | bisphosphatase 3 | |
| | | | | 61.9848 | Q9H7D7_LDNNLDS | Q9H7D7 | WDR26 WD repeat- | C338 |
| | | | | | VSLLIDHVCSR | | containing protein 26 | |
| 15.05 | | | | 61.9 | Q7RTR2_TQVAELL | Q7RTR2 | NLRC3 Protein | C553, |
| | | | | | QGCLRPDAAVCAR | | NLRC3 | C561 |
| | | | | 61.84 | Q9NUQ8_VAFAQM | Q9NUQ | ABCF3 ATP-binding | C628 |
| | | | | | TMPCPNFYILDEPT | 8 | cassette sub-family F | |
| | | | | | NHLDMETIEALGR | | member 3 | |
| | | | | 61.8176 | O95999_CSSCEPFPD | O95999 | BCL 10 B-cell | C119, |
| | | | | | GATNNLSR | | lymphoma/leukemia | C122 |
| | | | | | | | 10 | |
| | | | | 61.77 | Q9BRX8_RPGCFLC | Q9BRX8 | FAM213A Redox- | C85 |
| | | | | | R | | regulatory protein | |
| | | | | | | | FAM213A | |
| 21.7995 | | | 91.5874 | 61.3807 | Q15084_EVIQSDSL | Q15084 | PDIA6 Protein | C55, C58 |
| | | | | | WLVEFYAPWCGHC | | disulfide-isomerase | |
| | | | | | QR | | A6 | |
| 7.07 | | | | 61.24 | P30101_ISDTGSAGL | P30101 | PDIA3 Protein | C57, C60 |
| | | | | | MLVEFFAPWCGHC | | disulfide-isomerase | |
| | | | | | K | | A3 | |
| 21.91 | | | | 61.05 | O60825_VFFVESVC | O60825 | PFKFB2 6- | C158 |
| | | | | | DDPDVIAANILEVK | | phosphofructo-2- | |
| | | | | | | | kinase/fructose-2,6- | |
| | | | | | | | bisphosphatase 2 | |
| | | | | 60.7059 | Q14145_SGVGVAVT | Q14145 | KEAP1 Kelch-like | C613 |
| | | | | | MEPCR | | ECH-associated | |
| | | | | | | | protein 1 | |
| 18.0491 | 44.1085 | 25.0674 | 19.3862 | 60.6269 | Q9Y4E8_ISTETEETE | Q9Y4E8 | USP15 Ubiquitin | C633, |
| | | | | | GSLHCCK | | carboxyl-terminal | C634 |
| | | | | | | | hydrolase 15 | |
| 93.57 | | | | 47.52 | Q9NQC3_YSNSALG | Q9NQC | RTN4 Reticulon-4 | C1101 |
| | | | | | HVNCTIK | 3 | | |
| 83.08 | | | | 34.81 | Q00765_NCMTDLLA | Q00765 | REEP5 Receptor | C18 |
| | | | | | K | | expression-enhancing | |
| | | | | | | | protein 5 | |
| 61.48 | | | | 22.19 | P16615_ECVQPATK | P16615 | ATP2A2 | C997 |
| | | | | | | | Sarcoplasmic/ | |
| | | | | | | | endoplasmic reticulum | |
| | | | | | | | calcium ATPase 2 | |
| 60.86 | | | | 44.96 | P45880_VCEDLDTS | P45880 | VDAC2 Voltage- | C210, |
| | | | | | VNLAWTSGTNCTR | | dependent anion- | C227 |
| | | | | | | | selective channel | |
| | | | | | | | protein 2 | |
| 60.18 | | | | 36.72 | O15228_CYTPLVYK | O15228 | GNPAT | C54 |
| | | | | | | | Dihydroxyacetone | |
| | | | | | | | phosphate | |
| | | | | | | | acyltransferase | |
| 59.19 | | | | 16.34 | Q92503_YLGDLTPL | Q92503 | SEC14L1 SEC14- | C258 |
| | | | | | QESCLIR | | like protein 1 | |
| 58.17 | | | | | Q92685_SGSAAQAE | Q92685 | ALG3 Dol-P- | C21 |
| | | | | | GLCK | | Man:Man(5)GlcNAc(2)- | |
| | | | | | | | PP-Dol alpha-1,3- | |
| | | | | | | | mannosyltransferase | |
| 57.9565 | 76.1325 | 74.7915 | 84.2304 | 56.7152 | P29728_CFLEQIDSA | P29728 | OAS2 2′-5′- | C361 |
| | | | | | VNIIR | | oligoadenylate | |
| | | | | | | | synthase 2 | |
| 54.11 | 82.6656 | 91.6209 | 32.8738 | 25.19 | P09211_ASCLYGQL | P09211 | GSTP1 Glutathione | C48 |
| | | | | | PK | | S-transferase P | |
| 53.78 | | | | 12.02 | Q9NXH9_GDQCCYS | Q9NXH | TRMT1 tRNA | C620, |
| | | | | | HSPPTPR | 9 | (guanine(26)-N(2))- | C621 |
| | | | | | | | dimethyltransferase | |
| 53.22 | | | | 21.36 | O00410_STACQMLV | O00410 | IPO5 Importin-5 | C682, |
| | | | | | CYAK | | | C687 |
| 50.89 | 98.3152 | 91.0892 | 90.8865 | 39.3137 | P19447_SGVIVLPCG | P19447 | ERCC3 TFIIH basal | C342 |
| | | | | | AGK | | transcription factor | |
| | | | | | | | complex helicase | |
| | | | | | | | XPB subunit | |
| 50.06 | | | | 11.18 | O14920_NNSCLSK | O14920 | IKBKB Inhibitor of | C464 |
| | | | | | | | nuclear factor kappa- | |
| | | | | | | | B kinase subunit beta | |
| 49.16 | | | | | Q9NVN8_TIESYCQ | Q9NVN | GNL3L Guanine | C89 |
| | | | | | DVLR | 8 | nucleotide-binding | |
| | | | | | | | protein-like 3-like | |
| | | | | | | | protein | |
| 48.65 | | | | | Q96JB2_DHCALVR | Q96JB2 | COG3 Conserved | C363 |
| | | | | | | | oligomeric Golgi | |
| | | | | | | | complex subunit 3 | |
| 48.53 | | | | | Q5VYS8_NSVLAQG | Q5VYS8 | ZCCHC6 Terminal | C690 |
| | | | | | PGATSSAANTCK | | uridylyltransferase 7 | |
| 47.9153 | | | | 23.4664 | P48735_NYDGDVQS | P48735 | IDH2 Isocitrate | C336 |
| | | | | | DILAQGFGSLGLMT | | dehydrogenase | |
| | | | | | SVLVCPDGK | | | |
| 47.5 | | | | 35.63 | Q9H2U1_DCAVLSAI | Q9H2U1 | DHX36 Probable | C977 |
| | | | | | IDLIK | | ATP-dependent RNA | |
| | | | | | | | helicase DHX36 | |
| 47.1209 | −65.5983 | 24.1339 | 4.29804 | 29.8929 | Q96RU2_NVGNTCW | Q96RU2 | USP28 Ubiquitin | C171 |
| | | | | | FSAVIQSLFQLPEFR | | carboxyl-terminal | |
| | | | | | | | hydrolase 28 | |
| 39.2015 | 99.489 | 94.3358 | 90.3525 | 18.7286 | P23458_CRPVTPSCK | P23458 | JAK1 Tyrosine- | C810, |
| | | | | | | | protein kinase JAK1 | C817 |
| 50.89 | 98.3152 | 91.0892 | 90.8865 | 39.3137 | P19447_SGVIVLPCG | P19447 | ERCC3 TFIIH basal | C342 |
| | | | | | AGK | | transcription factor | |
| | | | | | | | complex helicase | |
| | | | | | | | XPB subunit | |
| 7.66 | 97.4228 | 95.8228 | −17.7122 | 25.7925 | P61088_ICLDILKDK | P61088 | UBE2N Ubiquitin- | C87 |
| | | | | | | | conjugating enzyme | |
| | | | | | | | E2 N | |
| 2.14646 | 94.9683 | 83.3324 | 95.8534 | 42.1898 | Q9NRW4_SCANPNV | Q9NRW | DUSP22 Dual | C124 |
| | | | | | GFQR | 4 | specificity protein | |
| | | | | | | | phosphatase 22 | |
| 21.3952 | 91.9716 | 95.872 | 19.6105 | 27.2983 | Q99836_DVLPGTCV | Q99836 | MYD88 Myeloid | C203 |
| | | | | | WSIASELIEK | | differentiation | |
| | | | | | | | primary response | |
| | | | | | | | protein MyD88 | |
| | 90.579 | 78.9223 | 82.8058 | 29.9 | Q9BRZ2_QLQGCPW | Q9BRZ2 | TRIM56 E3 | C338, |
| | | | | | APGPAPCLLPQLEL | | ubiquitin-protein | C347 |
| | | | | | HPGLLDK | | ligase TRIM56 | |
| 20.4722 | 89.3453 | 74.7494 | 53.869 | 24.0868 | Q99836_FITVCDYT | Q99836 | MYD88 Myeloid | C274, |
| | | | | | NPCTK | | differentiation | C280 |
| | | | | | | | primary response | |
| | | | | | | | protein MyD88 | |
| −24.3435 | 89.0944 | 81.7564 | 90.3507 | 26.69 | Q16875_AFFIESVCD | Q16875 | PFKFB3 6- | C155 |
| | | | | | DPTVVASNIMEVK | | phosphofructo-2- | |
| | | | | | | | kinase/fructose-2,6- | |
| | | | | | | | bisphosphatase 3 | |
| −7.60073 | 85.876 | 48.4428 | 82.1728 | 31.94 | P23743_DVVCLNDV | P23743 | DGKA | C95 |
| | | | | | SCYFSLLEGGRPED | | Diacylglycerol kinase | |
| | | | | | K | | alpha | |
| 2.41812 | 84.7455 | 4.7619 | 76.6489 | 25.6002 | Q13191_CTGPLANS | Q13191 | CBLB E3 ubiquitin- | C686 |
| | | | | | LSEK | | protein ligase CBL-B | |
| 54.11 | 82.6656 | 91.6209 | 32.8738 | 25.19 | P09211_ASCLYGQL | P09211 | GSTP1 Glutathione | C48 |
| | | | | | PK | | S-transferase P | |
| 32.3386 | 81.9213 | 67.2117 | 95.1871 | 41.8585 | Q9UPT9_ITSNCTIGL | Q9UPT9 | USP22 Ubiquitin | C171 |
| | | | | | R | | carboxyl-terminal | |
| | | | | | | | hydrolase 22 | |
| 14.7887 | 78.2704 | 43.8185 | 89.696 | 37.2032 | Q9H1C4_PCPYEQA | Q9H1C4 | UNC93B1 Protein | C583 |
| | | | | | QGGDGPEEQ | | unc-93 homolog B1 | |
| 10.83 | 77.833 | 57.4354 | 36.5844 | 32.73 | Q6L8Q7_SRPNASGG | Q6L8Q7 | PDE12 2′,5′- | C108, |
| | | | | | AACSGPGPEPAVFC | | phosphodiesterase 12 | C119 |
| | | | | | EPVVK | | | |
| 57.9565 | 76.1325 | 74.7915 | 84.2304 | 56.7152 | P29728_CFLEQIDSA | P29728 | OAS2 2′-5′- | C361 |
| | | | | | VNIIR | | oligoadenylate | |
| | | | | | | | synthase 2 | |
| 28.7525 | 72.8049 | 50.0993 | 84.5074 | 16.62 | Q06124_QGFWEEFE | Q06124 | PTPN11 Tyrosine- | C259 |
| | | | | | TLQQQECK | | protein phosphatase | |
| | | | | | | | non-receptor type 11 | |
| 9.5 | 66.0263 | 55.1344 | 79.9393 | 3.52504 | Q9UPT9_AIYQCFV | Q9UPT9 | USP22 Ubiquitin | C44 |
| | | | | | WSGTAEAR | | carboxyl-terminal | |
| | | | | | | | hydrolase 22 | |
| 12.0584 | 62.6861 | 54.2224 | 42.8553 | 26.32 | O14933_IYHPNVDE | O14933 | UBE2L6 | C86, C98 |
| | | | | | NGQICLPIISSENWK | | Ubiquitin/ISG15- | |
| | | | | | PCTK | | conjugating enzyme | |
| | | | | | | | E2 L6 | |
| −0.32817 | 58.3081 | −14.9577 | 24.795 | −38.85 | P24752_QAVLGAGL | P24752 | ACAT1 Acetyl-CoA | C119 |
| | | | | | PISTPCTTINK | | acetyltransferase, | |
| | | | | | | | mitochondrial | |
| 13.6326 | 57.1888 | 58.1986 | 73.3713 | 42.3797 | P40763_QQIACIGGP | P40763 | STAT3 Signal | C259 |
| | | | | | PNICLDR | | transducer and | |
| | | | | | | | activator of | |
| | | | | | | | transcription 3 | |
| | | 97.2429 | 94.695 | 48.523 | P41226_APASAAAS | P41226 | UBA7 Ubiquitin-like | C599 |
| | | | | | EDAPYPVCTVR | | modifier-activating | |
| | | | | | | | enzyme 7 | |
| 42.9641 | 41.5385 | 66.0276 | 68.8058 | 32.63 | Q16822_CLHSVGQP | Q16822 | PCK2 | C210, |
| | | | | | LTGQGEPVSQWPC | | Phosphoenolpyruvate | C230 |
| | | | | | NPEK | | carboxykinase | |
| 9.91709 | 51.6288 | 65.6292 | 29.0675 | 4.72 | Q9NT62_TVTIENHP | Q9NT62 | ATG3 Ubiquitin- | C264 |
| | | | | | HLPPPPMCSVHPCR | | like-conjugating | |
| | | | | | | | enzyme ATG3 | |
| 14.99 | 42.2981 | 65.3062 | 56.3522 | 24.29 | P29728_TFLKENCFR | P29728 | OAS2 2′-5′- | C381 |
| | | | | | | | oligoadenylate | |
| | | | | | | | synthase 2 | |
| −23.2607 | 36.6512 | 64.7718 | 35.5788 | 47.31 | Q9BRZ2_ITGLCPFG | Q9BRZ2 | TRIM56 E3 | C514 |
| | | | | | PR | | ubiquitin-protein | |
| | | | | | | | ligase TRIM56 | |
| 21.5095 | −16.5312 | 20.6349 | 93.1176 | 39.3137 | Q99873_VIGIECSSIS | Q99873 | PRMT1 Protein | C109 |
| | | | | | DYAVK | | arginine N- | |
| | | | | | | | methyltransferase 1 | |
| 2.85 | 19.0164 | 31.6334 | 85.444 | 47.95 | P29728_EWLSSPCF | P29728 | OAS2 2′-5′- | C668 |
| | | | | | K | | oligoadenylate | |
| | | | | | | | synthase 2 | |
| 13.05 | 27.2252 | −14.2894 | 80.9219 | 22.92 | Q14790_VFFIQACQ | Q14790 | CASP8 Caspase-8 | C360 |
| | | | | | GDNYQK | | | |
| 17.57 | 46.07 | 40.4424 | 80.6387 | 37.6635 | Q96RU2_CLSSEHAV | Q96RU2 | USP28 Ubiquitin | C733 |
| | | | | | IVK | | carboxyl-terminal | |
| | | | | | | | hydrolase 28 | |
| 15.5381 | 44.6347 | 32.9568 | 74.5258 | 10.0951 | P21580_AGNDAPAG | P21580 | TNFAIP3 Tumor | C590 |
| | | | | | CLSQAAR | | necrosis factor alpha- | |
| | | | | | | | induced protein 3 | |
| 13.6326 | 57.1888 | 58.1986 | 73.3713 | 42.3797 | P40763_QQIACIGGP | P40763 | STAT3 Signal | C259 |
| | | | | | PNICLDR | | transducer and | |
| | | | | | | | activator of | |
| | | | | | | | transcription 3 | |
| 15.7522 | 16.4621 | 44.2789 | 71.564 | 31.9527 | P43403_DNLLIADIE | P43403 | ZAP70 Tyrosine- | C346 |
| | | | | | LGCGNFGSVR | | protein kinase ZAP- | |
| | | | | | | | 70 | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods within the scope of these claims and their equivalents be covered thereby.

LENGTHY TABLES
The patent application contains a lengthy table section. A copy of the table is available in electronic form from the USPTO web site (https://seqdata.uspto.gov/?pageRequest=docDetail&DocID=US20240123078A1). An electronic copy of the table will also be available from the USPTO upon request and payment of the fee set forth in 37 CFR 1.19(b)(3).

What is claimed is:

1. A method of modulating an immune response in a subject in need thereof, comprising administering to the subject a therapeutically effective amount of a small molecule fragment of Formula (I): 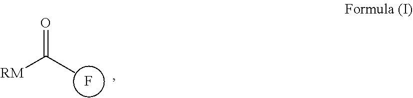
wherein:
RM is a reactive moiety selected from a Michael acceptor moiety, a leaving group moiety, or a moiety capable of forming a covalent bond with the thiol group of a cysteine residue; and
F is a small molecule fragment moiety.

2. The method of claim 1, wherein the small molecule fragment interacts with an endogenous cysteine-containing polypeptide expressed in the subject to form a cysteine-containing polypeptide-small molecule fragment adduct.

3. The method of claim 1 or 2, wherein the small molecule fragment is covalently bond to a cysteine residue of the cysteine-containing polypeptide.

4. The method of claim 2, wherein the cysteine-containing polypeptide-small molecule fragment adduct induces an immune response.

5. The method of claim 2 or 4, wherein the cysteine-containing polypeptide-small molecule fragment adduct induces a humoral immune response or a cell mediated immune response.

6. The method of claim 2, wherein the cysteine-containing polypeptide-small molecule fragment adduct increases an immune response relative to a control.

7. The method of claim 6, wherein the control is the level of an immune response in the subject prior to administration of the small molecule fragment or the level of an immune response in a subject who has not been exposed to the small molecule fragment.

8. The method of claim 2, wherein the cysteine-containing polypeptide is overexpressed in a disease or condition.

9. The method of claim 2, wherein the cysteine-containing polypeptide comprises one or more mutations, optionally overexpressed in a disease or condition.

10. The method of claim 8 or 9, wherein the disease or condition is cancer.

11. The method of claim 2, wherein the cysteine-containing polypeptide comprises a biologically active cysteine site, optionally located about 10 Å or less to an active-site ligand or residue.

12. The method of claim 2, wherein the cysteine-containing polypeptide is at most 50 amino acid residues in length.

13. The method of claim 1, wherein F is a small molecule fragment moiety illustrated in Table A.

14. The method of claim 1, wherein F is a fragment of the chloroacetamide compound in Table A after the chlorine (Cl) has been removed, or a fragment of the acrylamide compound after the C═C has been converted to an ethylene.

15. The method of claim 1, wherein the method modulates T cell activation.

16. The method of claim 1, wherein the method suppresses T cell activation.

17. The method of claim 1, wherein F optionally comprises a second reactive moiety.

18. The method of claim 1, wherein the method is an in vivo method.

19. The method of claim 1, wherein the endogenous cysteine-containing polypeptide is selected from proteins described in Tables 1 and 2.

20. A protein-probe adduct wherein the probe bind to a cysteine residue illustrated in Tables 1 and 2, wherein the probe has a structure represented by Formula (II): 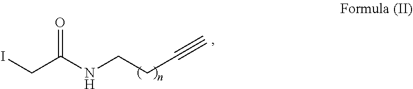
wherein n is 0-8.

21. A synthetic ligand that inhibits a covalent interaction between a protein and a probe, wherein the absence of the synthetic ligand and the probe binds to a cysteine residue illustrated in Tables 1 and 2; and wherein the probe structure is represented by Formula (II): 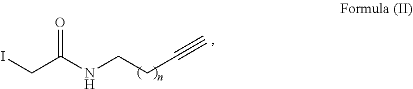
wherein n is 0-8.

22. A protein binding domain, wherein said protein binding domain comprises a cysteine residue illustrated in Tables 1 and 2, wherein said cysteine forms an adduct with a compound of Formula (II): 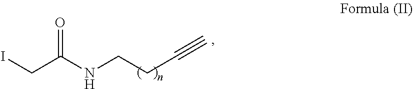
and wherein a compound of Formula (IIIA) or Formula (IIIB) interferes with the formation of the cysteine adduct by the compound of Formula (II), wherein Formula (IIIA) or Formula (IIIB) have the structure: 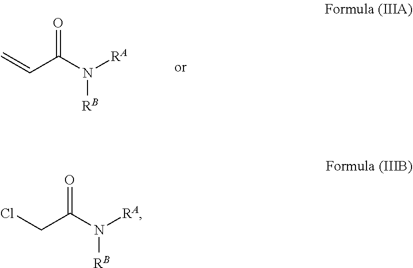
wherein,
each R^{A} and R^{B} is independently selected from the group consisting of H, D, substituted or unsubstituted C₁-C₆alkyl, substituted or unsubstituted C₁-C₆fluoroalkyl, substituted or unsubstituted C₁-C₆heteroalkyl, substituted or unsubstituted C₃-C₈cycloalkyl, substituted or unsubstituted C₂-C₇heterocycloalkyl, substituted or unsubstituted aryl, substituted or unsubstituted C₁-C₃alkylene-aryl, substituted or unsubstituted heteroaryl, and substituted or unsubstituted C₁-C₃alkylene-heteroaryl;
or R^{A} and R^{B} together with the nitrogen to which they are attached form a 5, 6, 7 or 8-membered heterocyclic ring A, optionally having one additional heteroatom moiety independently selected from NR¹, O, or S; wherein A is optionally substituted; and
R¹ is independently H, D, substituted or unsubstituted C₁-C₆alkyl, substituted or unsubstituted C₁-C₆fluoroalkyl, substituted or unsubstituted C₁-C₆heteroalkyl, substituted or unsubstituted aryl, or substituted or unsubstituted heteroaryl

23. A pharmaceutical composition comprising:
a) a cysteine-containing polypeptide covalently bond to a small molecule fragment, wherein the small molecule fragment is a small molecule fragment of Formula (I): 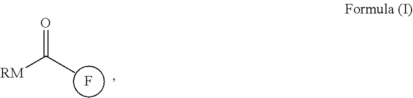
wherein;
RM is a reactive moiety selected from a Michael acceptor moiety, a leaving group moiety, or a moiety capable of forming a covalent bond with the thiol group of a cysteine residue; and
F is a small molecule fragment moiety; and wherein the small molecule fragment is covalently bond to a cysteine residue of the cysteine-containing polypeptide; and
b) an excipient.
